(19) United States
(12) Patent Application Publication
(10) Pub. No.: US 2026/0263377 A1
(43) Pub. Date: Sep. 10, 2026

(54) POLYMERIC BILE ACID ESTER NANOPARTICLES TO INDUCE TOLERANCE

(71) Applicant: Yale University, New Haven, CT (US)

(72) Inventors: Jung Seok Lee, New Haven, CT (US); Tarek M. Fahmy, Middlefield, CT (US); Dongin Kim, Glastonbury, CT (US)

(21) Appl. No.: 19/352,883

(22) Filed: Oct. 8, 2025

Related U.S. Application Data
(63) Continuation of application No. 16/907,055, filed on Jun. 19, 2020, now abandoned.
Application No. 16/907,055 is a continuation-in-part of application No. 15/757,608, filed on Sep. 2, 2016, now Pat. No. 10,864,170.
(60) Provisional application No. 62/214,648, filed on Sep. 4, 2015.

(51) Int. Cl. A61K 9/51; A61K 9/00; A61K 31/436; A61K 35/413; A61K 38/28; A61K 39/00; A61K 47/28; A61P 3/10; A61P 37/08

(52) U.S. Cl. CPC A61K 9/5153; A61K 31/436; A61K 35/413; A61K 38/28; A61K 39/0008; A61K 47/28; A61P 3/10; A61P 37/08; A61K 9/0053; A61K 2039/55555

(57) ABSTRACT

Polymeric bile acid (pBA) nanoparticles and tolerogenic formulation containing polymeric bile acid nanoparticles for oral delivery and induction of antigen-specific tolerance in a subject may include immunosuppressants and/or disease-specific antigen. Oral delivery results in local organ accumulation as well as systemic delivery of the nanoparticles. Early intervention with the nanoparticles induces antigen-specific tolerance and prevents development of autoimmune disorders. Treatment with the nanoparticles results in long-term antigen-specific immune tolerance, even after cessation of treatment, in autoimmune diseases.

### CROSS-REFERENCE TO RELATED APPLICATIONS

This application is a Continuation of U.S. application Ser. No. 16/907,055, filed Jun. 19, 2020, which is continuation-in-part of U.S. application Ser. No. 15/757,608, filed Mar. 5, 2018, which is a National Phase application under 35 U.S.C. § 371 of International Application No. PCT/US2016/050291, filed Sep. 2, 2016, which claims priority to and benefit of U.S. Provisional Application No. 62/214,648 filed Sep. 4, 2015, which are hereby incorporated herein by reference in their entireties.

### STATEMENT REGARDING FEDERALLY SPONSORED RESEARCH

This invention was made with government support under 0747577 awarded by National Science Foundation and under AI056363, CA199004, and CA026412 awarded by National Institutes of Health. The government has certain rights in the invention.

### FIELD OF THE INVENTION

The invention is generally directed to polymeric bile acid ester nanocompositions containing immunomodulators and/or antigen, which can be used to induced antigen-specific immune tolerance.

### BACKGROUND OF THE INVENTION

Direct priming of dendritic cells (DCs) with antigen and adjuvant is well established as a powerful vaccination approach for priming immunity. As such, biodegradable nanoparticles are promising vaccine vehicles with demonstrated applications in infection and cancer. One particular property of nanoparticles attractive for immunotherapy is their propensity to be taken up by antigen-presenting cells and the possibility of preferential targeting of professional antigen presenting cells, DCs, for delivery of protein antigens together with an immunogenic adjuvant.

However, nanoparticulate mediated tolerance induction is not as well understood due to nanoparticle mediated inflammatory response. While previous work has demonstrated the promise of nanoparticle-mediated delivery of antigen and immunosuppressive agent for immune tolerance in allergy, little is known about the mechanisms underlying how these systems function on a cellular and tissue level and therefore how they can be tailored appropriately towards development of new autoimmunity treatment options.

Another challenge is achieving antigen-specific induction of immune tolerance by oral delivery instead of injection. Delivery of active agents and/or imaging agents to internal organs following oral administration remains a challenge as the harsh biochemical environment inherent to the stomach, specifically the highly acidic pH and the presence of proteolytic enzymes, degrades and inactivates many therapeutic agents. Materials for forming oral drug delivery vehicles are carefully chosen to protect the active agents from the harsh conditions in the stomach and for a particular desired mode of agent release. Typically, the materials are not chosen to exert therapeutic effect on the target organ or cells in addition to the effect of the therapeutic agent.

There remains a need for improved oral delivery systems that utilize the delivery vehicles as therapeutics as well as increase the bioavailability and/or the efficacy of orally delivered agents for inducing antigen-specific tolerance.

Therefore, it is an object of the invention to provide a highly efficient oral delivery system for inducing antigen-specific immune tolerance.

It is yet another object of the present invention to provide methods of making the highly efficient oral delivery systems.

It is yet another object of the present invention to provide methods of using the highly efficient oral delivery systems.

### SUMMARY OF THE INVENTION

Polymeric bile acid ester (pBA) nanoparticles and tolerogenic formulations containing polymeric bile acid ester nanoparticles for inducing antigen-specific tolerance in a subject are typically formed of bile acid esterified polymers (pBA) having a molecular weight between about 800-1,000 (two monomers) and 240,000 Dalton (Da) (preferably approximately 400 monomers). The bile acid ester polymers are typically formed of one or more polymeric ursodeoxycholic acid (pUDCA), polymeric lithocholic acid (pLCA), polymeric deoxycholic acid (pDCA), polymeric chenodeoxycholic acid (pCDCA), and polymeric cholic acid (pCA). The bile acid ester polymers may be linear and/or branched polymers. References to pUDCA are generally applicable to other bile acid ester polymers. The nanoparticles formed of pBA may have diameters between 60 nm and 600 nm, more preferably between 100 nm and 400 nm, with a typical average geometric diameter of 350 nm. The polymeric nanoparticles may include other biocompatible polymer, as blends or as copolymers. In some embodiments, the nanoparticles are formed of pUDCA having a molecular weight between about 800 and 5,000 Da and having between about two and 20 UDCA monomeric units per polymer.

Typically, the bile acid ester polymers form a surface on the nanoparticles containing between 100 and 5000 bile acid monomeric units. The nanoparticles typically have at least 1.5 fold greater affinity, and up to about 50 fold greater affinity, to bile acid receptors than the respective monomers forming the bile acid ester polymers. The bile acid receptors include the G protein-coupled bile acid receptor 1 (GPBAR1 or Takeda G-protein receptor 5 (TGR5)) and the Farnesoid-X-Receptor (FXR). These receptors are placed at the interface of the host immune system with the intestinal microbiota and are highly represented in cells of innate immunity such as intestinal and liver macrophages, dendritic cells and natural killer T cells are generally on the surface of innate immune cells, such as macrophages.

Typically, the nanoparticles and/or the formulation contain one or more immunosuppressants, such as rapamycin (sirolimus) and analogs of rapamycin such as everolimus, ridaforolimus, remsirolimus, umirolimus, and zotarolimus. An immunostimulant may be enclosed, encapsulated, and/or associated with the nanoparticle. The nanoparticles and/or the formulation also typically contain a disease or disorder-specific antigen(s). The disease-specific antigen(s) may be enclosed, encapsulated, and/or associated with the nanoparticle.

Methods of inducing antigen-specific tolerance with the nanoparticles in a subject with an autoimmune or allergic disease typically include orally administering to the subject an effective amount of the nanoparticles. Typically, the nanoparticles distribute to internal organs, such as the heart, kidneys, spleen, lungs, liver, colon, and pancreas, following oral administration. This distribution is typically mediated by particles' intestinal transport and permeation through intestinal epithelium assisted by macrophage engulfment (by binding to TGR-5, endocytosis, exocytosis) and enterohepatic circulation (gall bladder accumulation and pancreatic ductal entry). This distribution is typically achieved in the absence of tissue- or organ-specific targeting agent.

Representative autoimmune and allergic diseases include type 1 diabetes, systemic lupus erythematous, rheumatoid arthritis, multiple sclerosis, food allergies, environmental allergies, and diseases with anti-drug antibodies (ADA). A method of treating type 1 diabetes is exemplified and includes orally administering to a subject in need thereof a formulation containing an effective amount pBA nanoparticles containing an anti-inflammatory and/or an immunosuppressive agent such as rapamycin.

The methods typically include administering the formulation for a period of at least one week, at least two weeks, or at least three weeks. In some embodiments, the formulation may be administered three times a week, two times a week, or once a day. Following treatment, a diabetic animal model may maintain healthy blood glucose for at least about three days, about five days, about one week, about two weeks, about one month, or more, after cessation of administering the formulation, and show an increase in the number of regulatory T cells (Treg) relative to a control. The subject may develop a tolerogenic phenotype.

The nanoparticles targeting pancreas, liver, or colon, deliver between 0.1 ng to 200 μg agent/NP of the agent to the target tissue, so that the total dosage is dependent upon the administered volume of NPs. The nanoparticles can release the agents over time, by sustained release, and/or in a single burst. For example, the one or more agent(s) encapsulated in the nanoparticles can be released over a period of time from between one hour and a few weeks, or can be released within the first 24 hours of reaching the target organ. Typical doses for treating inflammatory and/or autoimmune diseases are between 0.1 mg/kg and 1000 mg/kg, such as between about 0.4 mg/Kg and about 400 mg/Kg, between about 50 mg/Kg and 1000 mg/Kg, or between about 100 mg/Kg and 500 mg/Kg.

Methods of making NPs using self-assembly and aggregation of bile acid ester polymers have been developed. Two methods for making the bile acid assemblies include fabrication of branched polymeric bile acid units (as opposed to linear chains), and encapsulation through guest/host interactions in cavities that form with such branched building blocks; and supramolecular self-assembly via fluorinated bile acid units. Fluorination introduces a “fluorophobic effect.” This is distinctly different from hydrophobic or hydrophilic interactions, and results in self-assembly into a complex larger structure without the need for special formulation.

The NPs can exhibit therapeutic and/or prophylactic effects on inflammation (e.g., for treating autoimmune diseases) and/or metabolic regulation (e.g., for controlling blood glucose level and weight). It is believed this is due to the pBA, and more preferably, pUDCA, binding to the TGR5 bile acid receptor. The binding avidity and affinity of NPs to the bile acid receptors is enhanced when compared to those of the respective bile acid monomers. The enhanced avidity and affinity is due to the polymerization of bile acids and surface properties of NPs exposing between 100 and 5000 bile acid monomeric units. This permits the use of NPs as therapeutics with increased potency and efficacy when compared to the use of bile acid monomers.

The inclusion of a therapeutic agent, such as an anti-inflammatory or an immunosuppressive agent, typically results in a more than additive therapeutic effect as the effect of pBA, and preferably, pUDCA, is increased with the effect of the encapsulated drug. This more than additive effect is demonstrated in the results with the examples on preventing and treating type 1 diabetes.

PBAs binding bile acid receptors activate an intracellular pathway that facilitates endogenous insulin secretion, energy metabolism, endogenous insulin receptor expression, and a host of other functions such as reduction in reactive oxygen species and reduction in pro-inflammatory signaling. In the embodiments where the particles encapsulate insulin, this anti-inflammatory effect on cells happens before the insulin is released from the particles and binds its receptors and regulates glucose.

Therefore, the bile acid particles naturally mimic the physiologic process and it is because of this biomimicry that they are able to achieve a more than additive effect with encapsulated insulin. Because the pBA NPs first engage the bile acid receptors and initiate intracellular signaling and then release the encapsulated agent, pBA NPs typically increase and enhance the effect of the encapsulated agent.

Therefore, polymerization of bile acids significantly increases their binding avidity and affinity to bile acid receptors and improves the therapeutic effect of bile acids. pBA NPs alone show therapeutic anti-inflammatory and immunosuppressive effects. Encapsulation of therapeutic agents enhances the therapeutic effect of the agent as this effect is increased with the action of pBAs. As shown in the examples, the NPs are therapeutic with broad-spectrum properties that manage T1D in the short-term and function to reverse pathology and restore endogenous insulin secretion and regulatory immunity in the long-term.

### BRIEF DESCRIPTION OF THE DRAWINGS

FIGS. 1A-1J are schematics of bile acid monomers and the polymeric BAs (pBA) formulated into NPs under emulsion conditions.

FIGS. 1A-1E are structures of cholic acid (CA) (FIG. 1A), chenodeoxycholic acid (CDCA) (FIG. 1B), deoxycholic acid (DCA), (FIG. 1C) lithocholic acid (LCA) (FIG. 1D), and ursodeoxycholic acid (UDCA) (FIG. 1E).

FIGS. 1F-1J show polymerization and formation of nanoparticles. Monomers are esterified at the carbon-24 position on monomeric BAs (FIG. 1F) to result in hydrolysable ester bonded BAs (pBAs) (FIG. 1G). The schematic of the polymerization step shows the location of the polymer-forming reactive end groups. Emulsification of pBAs (FIG. 1H) in the presence of drug yields drug entrapped in solid pBA NPs (FIG. 1I) with an average diameter of 344.3±4.7 nm (FIG. 1J).

FIG. 2 is a schematic of bond correlations in pUDCA revealed by Key HMBC and COSY NMR spectra. Given the intensities of ¹H NMR signals which provide information on the relative number of protons, NMR data of pUDCA reveals that two hydroxyl substituents at C-3 and C-7 are esterified with 2.5:1 molar ratio during the polymerization process.

FIGS. 3A-3N are graphs showing distribution and uptake of polymer bile acids (pBAs) in vitro and in vivo.

FIG. 3A is a graph (total NP as % initial dose/cn for pUDCA and control PLGA NPs in the biodistribution in non-gastrointestinal organs, heart, kidneys, spleen, lungs, liver, and pancreas.

FIG. 3B is a graph of the dye-independent localization of NP in the pancreas. Pancreatic accumulation of NPs was quantitated when coumarin 6 was used as a tracer to confirm that the level of pancreatic accumulation of NP was independent of the physiochemical properties of the loaded agent, but dependent on the particle composition. Free coumarin was dispersed in 1% TWEEN® 20 in saline.

FIG. 3C is a graph of cell viability (%) to show cytotoxicity of NPs (1 mg/mL) in Caco-2 cells (10⁴ cells/well) and BMMs (10⁴ cells/well) measured using a CellTiter-Blue® Cell Viability Assay (Promega Co.) after incubation at 37° C. for 24 h for PLGA, pGA, plGA, pDCA, pCDCA, and pUDCA.

FIG. 3D is a graph of interferon gamma when OT-II T cells were cocultured with pUDCA-treated dendritic cells (DCs) that were stimulated by lipopolysaccharide (LPS) and ovalbumin (OVA), showing saline and OVA controls, OVA and pUDCA at 50 and 5 micrograms/ml, respectively. Decrease in the interferon gamma (IFNγ) level from OT-II CD4+ T cells were measured.

FIG. 3E is a graph of the impact of pUDCA compare compared to UDCA and PLGA on secretion of pro-inflammatory cytokine, IL-6 (pg/ml), from macrophages.

FIG. 3F is a graph of particle size under simulated stomach conditions (nm) for PLGA, PLGA/pUDCA and pUDCA. Particle stability was evaluated by measuring particle sizes over time in the simulated stomach conditions (citrate buffer solution, pepsin 10 mg/mL, pH 2.0, 37° C.).

FIG. 3G is a graph of the % bioactivity of released insulin from pUDCA NPs. The released insulin from pUDCA at 3 or 24 h was incubated with CHO INSR cells for 1 h and pAkt was measured by ELISA. The pAkt production from CHO INSR cells that were incubated with fresh or denatured insulin was measured to calculate percent bioactivity. The average bioactivity of released INS was 87.3% of fresh insulin.

FIG. 3H is a graph of the permeability of NPs formed of PLGA, pGA, plGA, pDCA, and pCDGA through a layer of Caco-2 cells on transwell filters.

FIG. 3I is a graph of pancreatic trafficking with and without macrophage depletion. B6 mice were depleted macrophages and treated with DIR-loaded pUDCA NPs by oral gavage (500 mg/kg, 250 L). Clodrosome (Clodronate-containing liposomes, 100 mg/kg, IP) was used to deplete macrophages. Pancreata were harvested at 4 h post gavage and imaged.

FIG. 3J is a graph of CD11c-F4/80+ macrophages associated with coumarin 6-loaded pUDCA NPs in pancreas, liver, lungs, and spleen in mice were acquired using a flow cytometer at 4 h post oral ingestion.

FIG. 3K is a graph of competitive binding of pUDCA and UDCA to TGR5 on macrophages at 4° C. as a function of concentration (micrograms/ml) pUDCA, UDCA, and PLGA.

FIG. 3L is a graph of the number of particles in cells (×10⁵) over time in hours, showing the rate of endocytosis 37° C. and exocytosis at 4° C. for pUDCA, PLGA/pUDCA, and PLGA. (**P<0.01 and ***P<0.001).

FIG. 3M is a graph showing insulin production (ng/ml) induced by pUDCA and UDCA from pancreatic β cells.

FIG. 3N is a graph showing IFN-7 production of CD4+ T cells, directly treated with pUDCA (50 and 5 micrograms/ml), and stimulated with anti-CD3 and anti-CD28.

FIGS. 4A-4I are graphs showing comparative prevention of T1D.

FIG. 4A shows an experimental scheme. Pancreatic inflammation was induced at day 0 with IP injection of cyclophosphamide (CY).

FIG. 4B is graph showing comparative assessment of formulations in prevention of T1D, as blood glucose (mg/dl), as a function of days post CY treatment with empty pUDCA (pUDCA_{EMPTY}), monomer UDCA (UDCA_{EMPTY}), pLCA (pLCA_{EMPTY}), and pDCA (pDCA_{EMPTY}) after oral gavage.

FIG. 4C is graph of the percent diabetic animals (glucose >200 mg/dL) post CY treatment.

FIG. 4D is a graph of the effect of pUDCA_{RAPA} on blood glucose levels.

FIG. 4E is a graph of the percent of animals that became diabetic. A single dose on day 1 is denoted Dose I and two doses on 2 consecutive days is denoted Dose II.

FIG. 4F is a graph of the post-dose reduction in CD8+ T cell frequency over 5 days, for saline, and pUDCA_{RAPA} dose I and dose II.

FIG. 4G is graph of normalized % CD8 T cells comparison at day 5 at saline, dose I, and dose II.

FIG. 4H is a graph of the post-dose enhancement in % Treg (CD4+CD25+FoxP3+) frequency over 5 days.

FIG. 4I is a graph of the normalized comparison at day 5 post inducation of inflammation. All experiments were performed with 10 samples/animals per group and repeated twice. (*P<0.05, **P<0.01, and ***P<0.001).

FIGS. 5A and 5B are graphs showing dose-dependent therapeutic efficacy in prevention of T1D with rapamycin-loaded pUDCA NPs (pUDCA_{RAPA}). The prophylactic effect of Rapa-loaded pUDCA was tested as a function of dose in CY induced T1D animal models. Doses were: 50, 100 and 500 mg/kg pUDCA. pUDCA was orally administered for two days (NP arrows) one day post CY induction (day 0, CY arrow). The results indicate a prophylactic effect (i.e. prevention of disease onset) that is dose-dependent as assessed by degree of blood glucose lowering (FIG. 5A) and percentage of animals that were non-diabetic after 30 days (FIG. 5B). The indicator for diabetes onset was a blood glucose level greater than 200 mg/dL.

FIGS. 6A-6O are graphs showing short-term treatment and long-term regression of T1D after pUDCA_{INS} NP oral ingestion, activation of TGR5 induced endogenous GLP-1 and insulin secretion, and anti-inflammatory effect of pUDCA.

FIG. 6A is a graph of the short-term blood glucose (mg/dL) over time in days following treatment with UDCA-insulin (500 mg/kg), pUDCA-insulin (100 mg/kg), pUDCA-empty (500 mg/kg), and pUDCA-insulin (500 mg/kg).

FIG. 6Bis a graph of short-term blood glucose (mg/dl) over time (days) for pUDCA compared to UDCA (n=10). Oral treatment commenced after glucose ≈200 mg/dL and was seven doses at 1 dose/day with either 100 or 500 mg/kg.

FIG. 6C is a graph of the long-term blood glucose (mg/dL) over time (days). Long-term reversal of spontaneous T1D disease after oral treatment with insulin loaded NPs (n=6).

FIG. 6D is a graph of the body weight (grams) over time (days), saline, soluble insulin, PLGA insulin, and pUDCA insulin.

FIG. 6E is a graph of the percent survival of diabetic mice over time in days showing the survival profile after pUDCA treatment with log-rank test and χ² statistical analysis (up to 90 days).

FIG. 6F is a graph of the blood glucose (mg/dl) in T1D for times (days) for saline, soluble oral, soluble insulin (subcutaneous), soluble insulin (intraperitoneal), and pUDCA insulin (oral).

FIG. 6G is a graph of blood glucose in swine (mg/dl) over time (day). T1D was induced in Ossabaw Swine by treatment with alloxan, then the animals were orally treated with pUDCA_{INS} 7 times. Blood glucose level was measured every 5 mins from three swine and averaged to compare to control swine received saline. Arrows indicate oral dosings and food resumption.

FIGS. 6H and 61 are graphs of serum insulin concentration (ng/ml) (FIG. 6H) and pancreatic insulin (FIG. 6I) at 4, 8, and 24 h post oral ingestion, for saline, free insulin, PLGA-insulin, pUDCA-insulin.

FIGS. 6J and 6K are graphs of GLP-1 secretion (pmol/L) (FIG. 6J) and insulin production (FIG. 6K), as a result of TGR5 activation by pUDCA.

FIGS. 6L and 6M are graphs of the % CD44+CD8+ T cells (FIG. 6L) and % Foxp3+CD25+CD4+ Tregs (FIG. 6M) following administration of saline and pUDCA-insulin. Pancreatic lymph node CD8+ T cell frequency and FIG. 6M, CD4+ Tregs (n=10).

FIG. 6N is graph of the IL-10 levels (pg/ml) and CCL1 (pg/ml) for pUDCA, UDCA, PLGA showing production of anti-inflammatory cytokine (IL10) and chemokine (CCL1).

FIG. 6O is a graph M1/M2 ratio for pUDCA, UDCA, PLGA, saline showing macrophage phenotype skewing from M1 (CD86) to M2 (CD206) induced by pUDCA. All experiments were performed with more than 6 samples/animals per group and repeated twice with exception of swine study (*P<0.05, **P<0.01, and ***P<0.001).

FIGS. 7A and 7B are experimental schemes for two groups of mice: Group A were used to detect the efficacy with OTii adoptive transfer (FIG. 7A), and Group B were used to evaluate efficacy in OTii Mice (no cell transfer, FIG. 7B). FIG. 7C is a graph showing percent of CD25+Foxp3+ of OTii cells obtained from animals treated with vehicle, pUDCA-OVA NPs (OVA NP), or pUDCA-OVA-RAPA NPs (OR NP).

FIG. 8A is an experimental scheme for therapeutic antigen-specific tolerance induction in a mouse model of type 1 diabetes.

FIGS. 8B and 8C show changes in blood glucose (mg/dL) in mice administered seven daily oral doses of saline, empty pUDCA NPs, pUDCA-BDC, or pUDCA-BDC-RAPA at high dose (10 mg/dose, FIG. 8B) or low dose (2 mg/dose, FIG. 8C).

FIGS. 8D and 8E are graphs showing change in percent CD44+CD8+ T cells (FIG. 8D) or in percent CD4+CD25+FoxP3+ Tregs (FIG. 8E) in diabetic untreated mice or mice treated with BDC/pUDCA-RAPA.

FIGS. 9A and 9B are bar graphs showing fasting blood glucose levels (mg/dL) at one month (FIG. 9A) and six months (FIG. 9B) after alloxan administration to juvenile and adult swine.

FIG. 10A is a graph showing changes in blood glucose level (mg/dL) in alloxan-induced diabetic swine after cumulative daily dosing for seven days of pUDCA and 0.01% insulin, 6.4 mg/kg dose (each daily dose delivered 6.4 mg/kg particles containing 0.01% insulin)

FIG. 10B is a graph showing changes in blood glucose level (mg/dL) in alloxan-induced diabetic swine after single dose of water gavage, pUDCA-insulin, or subcutaneous insulin 70/30. pUDCA-insulin particles produced wider trough and a reduced post-prandial effect with a mean blood glucose level of 65 mg/dL.

FIG. 10C is a graph showing changes in blood glucose level (mg/dL) in alloxan-induced diabetic swine after repeat daily dosing with subcutaneous insulin for four days, followed by a single dose of pUDCA (upper arrow). Bottom arrow shows a possible post-prandial effect in the absence of any external insulin. A single pUDCA administration eliminated the need for insulin for the next three days.

FIG. 10D is a bar graph showing change in baseline fasting glucose (mg/dL) over time (days) for adult alloxan-induced diabetic swine two weeks or one month after pUDCA treatment. Insulin-loaded pUDCA NPs rapidly reversed alloxan-induced diabetes in adult Ossabaw swine.

FIG. 10E is a diagram showing the effect of pUDCA NPs in diabetes therapy. NPs offer diabetic care and treatment from three points: oral delivery with good bioavailability for treating late stage T1D and T2D, metabolic restoration for treating early stage T1D, and reduction in autoimmune reactivity for early stage T1D.

FIG. 11A is an experimental scheme for therapeutic antigen-specific tolerance induction in a mouse model of multiple sclerosis.

FIG. 11B is a graph showing change in clinical score over time (days) in mice left untreated, or treated with soluble MOG, pUDCA-MOG, soluble MOG/Rapa, or pUDCA-MOG/Rapa.

FIG. 12A is an experimental scheme for therapeutic antigen-specific tolerance induction in a mouse model of collagen-induced arthritis (CIA).

FIG. 12B is a graph showing change in clinical score over time (days) in mice with CIA: untreated (vehicle), or treated with soluble MOG, empty pUDCA, pUDCA-Rapa, pUDCA-Collagen, or pUDCA-Collagen-Rapa.

### DETAILED DESCRIPTION OF THE INVENTION

### I. Definitions

As used herein, the term “nanoparticle” generally refers to a particle having a diameter from about 10 nm up to, but not including, about 1000 nm, preferably from about 60 nm to about 450 nm. The particles can have any shape. Typically, the nanoparticles are spherical and the size is presented as diameter measured in nm as the geometric mean.

As used herein, the term “encapsulated” refers to the agent, for example, a therapeutic and/or an imaging agent, encapsulated within, surrounded by, and/or dispersed throughout a polymeric matrix of the nanoparticle. Alternatively or additionally, the agent can be associated with a polymeric matrix by hydrophobic interactions, charge interactions, van der Waals forces, etc.

As used herein, the term “untargeted” refers to nanoparticles formed of a polymer, such as pUDCA or PLGA, without additional elements, such as targeting moieties, having an increased affinity to a particular cell type or organ. As used herein, the term “targeting moiety” refers to any molecule such as an antibody, ligand, receptor binding moiety, or an active fragment thereof, or an agonist, antagonist, or tissue- or cell-specific targeting molecule, that is used to attach the nanoparticle to a cell in the target organ.

As used herein, the term “active agent” or “biologically active agent” are used interchangeably to refer to a chemical or biological compound that induces a desired pharmacological and/or physiological effect, wherein the effect may be prophylactic, therapeutic and/or diagnostic. The terms also encompass pharmaceutically acceptable, pharmacologically active derivatives of active agents, including, but not limited to, salts, esters, amides, prodrugs, active metabolites, and analogs.

As used herein, the term “excipient”, or “pharmaceutically acceptable excipient”, refers to a pharmacologically inactive substance added to the composition to further facilitate administration of the composition.

As used herein, “oral administration” refers to delivery of the composition to a subject via an oral route. Oral administration can be achieved via oral gavage, or by swallowing of the composition in liquid or solid form. The liquid forms of orally administered compositions can be in a form of a solution, emulsion, suspension, liquid capsule or a gel. Solid forms of orally administered compositions include capsules, tablets, pills, powders, and granules.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, “tolerance” means the reduction in the ability of the immune system to mount an adaptive (T or B-mediated) response to a given antigen.

As used here, “tolerogenic” means the condition or capability of stimulating or increasing tolerance.

As used herein “Treg” includes any T cell that confers suppression. Thus the term encompasses traditional CD4, Foxp3+ Tregs, as well as other CD4 cells that do not express Foxp3 but can be regulatory by secreting IL-10 (Tr1 cells) among other signals, and CD8 Tregs (Foxp3+ and −) which have also been identified.

### II. Compositions

The compositions include nanoparticles formed of poly(bile acid) ester polymers. These particles are administered in the absence of therapeutic, prophylactic and/or diagnostic agents incorporated therein or thereon, and, optionally, pharmaceutically acceptable excipients.

Bile acids have been used for decades to enhance oral uptake of drugs. See, for example, Samstein, et al. Biomaterials 29 (2008) 703-708. Bile salts were used to improve the bioavailability of poly(lactide-co-glycolide) (PLGA) nanoparticles by protecting them during their transport through the gastrointestinal tract and enhancing their absorption by the intestinal epithelia. A deoxycholic acid emulsion was shown to protect PLGA nanoparticles from degradation in acidic conditions and enhance their permeability across a model of human epithelium. Oral administration of loaded PLGA nanoparticles to mice, using a deoxycholic acid emulsion, produced sustained levels of the encapsulant in the blood over 24-48 h with a relative bioavailability of 1.81. Encapsulant concentration was highest in the liver, demonstrating targeted delivery to the liver by the oral route.

Studies have now demonstrated that not only does the use of bile acid ester polymers, such as pUDCA, significantly enhance uptake orally, but that the empty particles have antiinflammatory properties. This is believed to be effected through binding of the polymers, e.g., pUDCA, to the TGR5 receptor. With the enhanced surface avidity due to the polymerization and spherical form, empty pUDCA NPs (i.e., not including added therapeutic or prophylactic agent) are effective in reducing inflammation, for example, for treatment of diabetes. Studies show upregulation of GLP-1 through TGR5 binding in the ileum. The anti-inflammatory aspects of UDCA are also magnified in a similar manner.

Based on these findings, the pUDCA NPs are expected and shown to be useful in treating autoimmune and inflammatory diseases and conditions of the pancreas, liver, and colon, including diabetes, pancreatitis, primary biliary cirrhosis (PBC), nonalcoholic steatohepatitis (NASH), IBD, and rCDI (Clostridioides difficile). Generally, the pUDCA NPs provide sustained release of UDCA from pUDCA as the ester bonds deteriorate.

### A. Polymers

Generally, the monomers of bile acids suitable for forming poly(bile acid) polymers, are defined by Formula I:

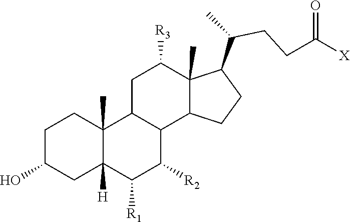

wherein:
R₁, R₂, and R₃ are independently hydrogen or hydroxyl group, and
X is a hydroxyl group at low pH (2-5) that is deprotonated at pH above 5.5. Optionally, X is NHCH₂COOH, NHCH₂COO—, NHCH₂CH₂SO₃H, or NHCH₂CH₂SO₃⁻, representing glycine or taurine conjugates (also known as bile salts) of the corresponding bile acid.

The fully protonated hydroxyl group at position X renders the monomers insoluble in water, and the loss of the proton improves the water solubility of the monomers.

The structure of bile acid monomer cholic acid (CA) is shown in Formula II:

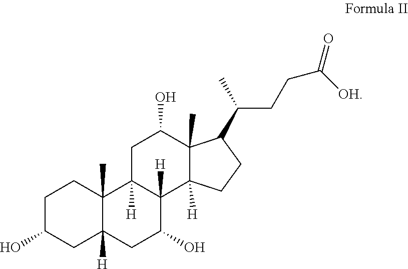

The structure of bile acid monomer lithocholic acid (LCA) is shown in Formula III:

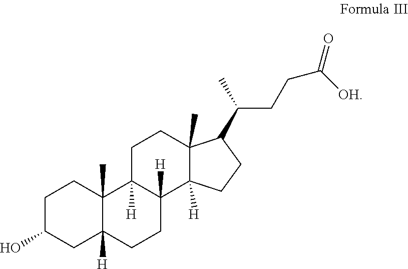

The structure of bile acid monomer deoxycholic acid (DCA) is shown in Formula IV:

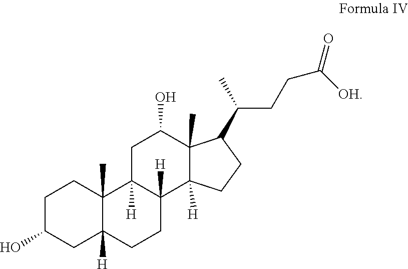

The structure of bile acid monomer cheno-deoxycholic acid (CDCA) is shown in Formula V:

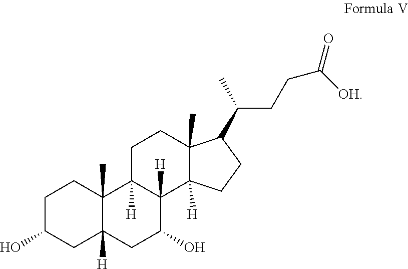

The structure of bile acid monomer urso-deoxycholic acid (UDCA) is shown in Formula VI:

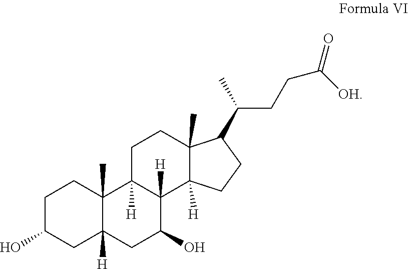

Other suitable bile acids include, but are not limited to, glycocholic acid, taurocholic acid, glycodeoxycholic acid, taurodeoxycholic acid, lithocholic acid, taurolithocholic acid, taurochenodeoxycholic acid, tauroursodeoxycholic acid, glycolithocholic acid, glycochenodeoxycholic acid, glycoursodeoxycholic acid, and taurine conjugates of 3-alpha-7-alpha-12-alpha-22-xi-tetrahydroxy-5-beta-cholestan-26-oic acid (tetrahydroxystero-cholanic acid) and 3-alpha-12 alpha-22-xi-trihydroxy-5-beta-cholestan-26-oic acid.

Other suitable bile acids also include muricholic acids (such as α-muricholic acid, β-muricholic acid, γ-muricholic acid, and ω-muricholic acid), hyodeoxycholic acid, ursocholic acid, isocholic acid, isodeoxycholic acid, isolithocholic acid, isochenodeoxycholic acid, isoursodeoxycholic acid, norcholic acid, nordeoxycholic acid, norlithocholic acid, norchenodeoxycholic acid, norursodeoxycholic acid, apocholic acid, allocholic acid, and their taurine or glycine conjugates.

Additional suitable bile acids are described in Heinken et al., Microbiome 2019, 7:75; Schmidt et al., J Biol Chem, 2010, 285(19):14486-94; Chiang, Compr Physiol, 2013, 3(3): 1191-1212; Sarenac and Mikov, Front Pharmacol, 2018, 9:939; de Haan et al., J Clin Transl Res, 2018, 4(1):1-46; LIPID MAPS Structure Database: Bile acids and derivatives (https://www.lipidmaps.org/data/structure/LMSDSearch.php?Mode=ProcessClassSearch&L MID=LMST04).

The above-listed monomers are esterified to produce the poly(bile acid) (PBA) polymers having a molecular weight between about 800 (at least two monomers) and 250,000 Daltons, more preferably between 800 and 50,000 Daltons. In some embodiments, the pUDCA polymers have an Mw value between about 1000 and about 10,000 Daltons, or between about 1200 and about 5,000 Daltons. Room temperature polymerization of bile acids can be carried out using a mixture of diisopropyl carbodiimide (DIC), and a 1:1 salt of dimethyl amino pyridine and p-toluenesulfonic acid (DMAP/PTSA) in mild reaction conditions and without significant cross-linking. Carboiimide activation leads to preferential esterification at carbon 3 and linear polymeric chains. Applied to UDCA, the polymerized UDCA can be defined by Formula VII:

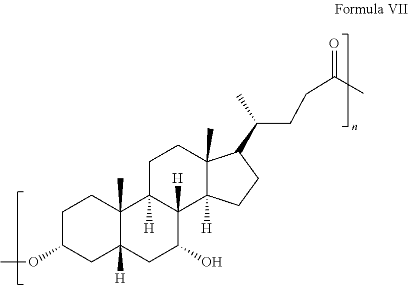

wherein n is a number ranging from between 2-600, preferably between 2 and 100, corresponding to a polymer Mw average in the range of 800-240,000 Daltons.

The degree of branching can vary from a generation 0 (no branches) to higher unlimited number of generations. An exemplary polymerized UDCA with branching is shown in Formula VIII:

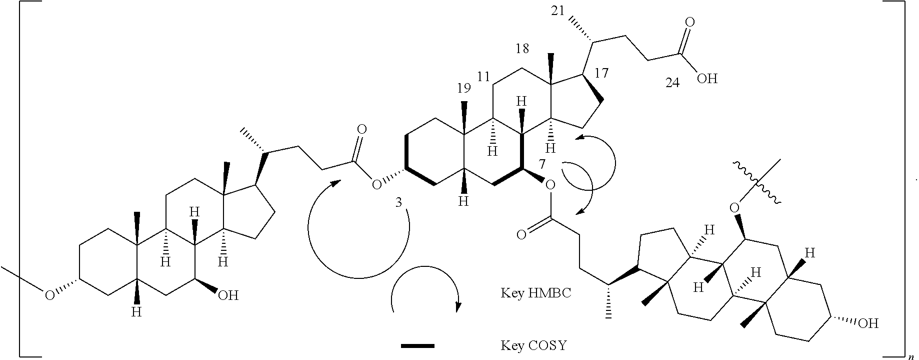

The polymers may be formed from the same monomer, such as UDCA, forming poly(UDCA), or PUDCA. In other embodiments, the polymers may be formed from a mix of bile acid monomers, forming copolymers or monomers coating a polymer bile acid core. In these embodiments, the monomers or polymers may be mixed in any combination, and at any ratio, to form polymeric blends of bile acid ester polymers ranging in molecular weight from between 800 and 250,000 Daltons. Typically, the polymers are linear, but other structures, such as branched, or forked, or dendrimeric, could be used. A dendrimer of poly(bile acids) (dendritic PUDCA, for example), will have a pH stimuli response similar to the linear chain counterparts. This dendritic system will be in a swollen or open state at physiological pH or pH above 6.0. Therefore, it can be easily loaded with drug through non-covalent association with the dendritic polymer or by entrapment in the interstitial cavities formed in the branched system. Low pH will shrink the system, protecting the encapsulant and/or releasing it more slowly. As such, a dendritic bile acid ester polymer may serve as a nanoparticle itself, without the formulation conditions used with linear polymers.

The pUDCA polymers can be formed of ursodeoxycholic acid, glycoursodeoxycholic acid, tauroursodeoxycholic acid, or a combination thereof.

In some embodiments, the monomers or the formed polymeric chains may include moieties with one or more radionuclides, or optical tracers (bioluminescent, chemiluminscent, fluorescent or other high extinction coefficient or high quantum yield optical tracers). Similarly, non-invasive contrast agents such as T1 MR agents in the class of heavy metals (gadolinium, dysprosium, etc.) or T2 contrast agents (iron oxide, manganese oxide, etc.), iodinated agents for X-ray attenuation (CT) and other modalities. The inherent ability of these systems to respond to changes in the pH range of 7 to 2 has significant implications for delivery of therapeutics both to low pH endocytic compartments within cells and/or sites of inflammation characterized by low pH microenvironment or the surrounding environment of tumors. The polymeric chains of these embodiments can be used to form traceable pUDCA nanoparticles, eliminating the need of encapsulating imaging/tracing agents, and enhancing the imaging modalities due to local retention of the imaging agent (confinement of the probe) in the area.

The pUDCA nanoparticles are pH responsive. The polymer backbone shrinks, and the nanoparticles aggregate, in a low pH microenvironment (pH 2-5), and expands at higher pH (pH 6-7.5) to release an encapsulated agent. The pUDCA polymer allows for encapsulation of both hydrophilic and hydrophobic drugs, peptides, proteins, and oligonucleotides. The encapsulated agents are released over time in the higher pH microenvironment of the gut lumen, or generally in organs with pH above 5.5-6.0.

The water solubility of bile acids rises exponentially with increasing pH (Hoffman et al., J. Lipid Res., 33:617-626 (1992)). The polymeric chains of pUDCA and nanoparticles made therefrom aggregate at low pH and become increasingly soluble/dispersed as the pH increases above 5.5. These polymers and nanoparticles are particularly suited for oral drug delivery, as they can protect the agent(s) encapsulated with the nanoparticles from the destructive environment of the stomach. The agent(s) can then be safely released at the neutral pH in the intestines (typically 6-7.4) and target organs, as the polymers begin to dissolve releasing the agent(s).

The nanoparticles can have a mean geometric diameter that is between 50 and 500 nm. In some embodiments, the mean geometric diameter of a population of nanoparticles is about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, the mean geometric diameter is between 100-400 nm, 100-300 nm, 100-250 nm, or 100-200 nm. In some embodiments, the mean geometric diameter is between 60-400 nm, 60-350 nm, 60-300 nm, 60-250 nm, or 60-200 nm. In some embodiments, the mean geometric diameter is between 75 and 250 nm. In some embodiments, 30%, 40%, 50%, 60%, 70%, 80%, 90%, or more of the nanoparticles of a population of nanoparticles have a diameter that is between 50 and 500 nm. In a preferred embodiment, the average particle size is 350 nm. Size is measured by conventional techniques, such as optical microscopy.

### B. Tolerogenic Compositions

Compositions to induce tolerance typically contain, or are formulated with or for co-administration with, tolerogenic (tolerizing) antigen, an immunosuppressant (e.g., rapamycin), or combination thereof, to dendritic cells or antigen presenting cells (APCs). In some embodiments, the tolerogenic antigen and the immunosuppressant are co-delivered to the same cells. APCs can then become tolerogenic and migrate to peripheral lymphoid lymph nodes where it is believed they activate, induce proliferation, induce differentiation, or combination thereof of Tregs such as CD4+Foxp3+ cells. These Tregs can then suppress activation and antibody production by B cells specific for the tolerogenic antigen. It is desirable that the antigen and immunosuppressive drug be spatially localized to the same liver dendritic cell or liver endothelial cell for initiation of the tolerogenic program. Therefore, in the most preferred embodiments, the antigen and immunosuppressive drug are loaded into, dispersed within, conjugated to, or otherwise displayed on or in same particle. Co-delivery of immunosuppressant with antigen in the same particle can have two effects: 1) concentrating the antigen and drug dose in the same cell, and 2) ensuring that the same antigen-presenting cells are suppressed. This strategy can reduce or prevent broad immunosuppression or antigen-specific immunogenicity.

Immunosuppressant is delivered with the antigen to the same antigen presenting cell to improve the immunosuppressive effect (e.g., tolerance induction) of the drugs. In some embodiments, two immunosuppressants are co-delivered, such as mycophenolic acid and rapamycin. Preferably the particles accumulate in the liver. In some embodiments, the particle includes a targeting moiety, for example a targeting moiety that increases (or further increases) the accumulation of the particle in the liver or directs the particles to specific cells, such as dendritic cells in the liver.

In alterative embodiments, the antigen and the immunosuppressive drug are loaded into, dispersed within, conjugated to, or otherwise displayed on or in separate particles.

### C. Antigens

The particles can include one or more antigens to which tolerance is to be induced. A suitable antigen is selected based on the desired therapeutic outcome and the disease, disorder, or condition being treated. Exemplary antigens are known in the art. See, for example, U.S. Published Application No. 2014/0356384 which discusses:

The tolerogenic antigen can be derived from a therapeutic agent protein to which tolerance is desired. Examples are protein drugs in their wild type, e.g., human factor VIII or factor IX, to which patients did not establish central tolerance because they were deficient in those proteins, nonhuman origin protein drugs, for administration to humans. Examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans include human proteins with engineered mutations, e.g., mutations to improve pharmacological characteristics. Examples of tolerogenic antigens that contain nonhuman glycosylation include proteins produced in yeast or insect cells.

The tolerogenic antigen can be derived from proteins that are administered to humans that are deficient in the protein. Deficient means that the patient receiving the protein does not naturally produce enough of the protein. The proteins may be proteins for which a patient is genetically deficient of which are dysfunctional. Such proteins include, for example, antithrombin-III, protein C, factor VIII, factor IX, growth hormone, somatotropin, insulin, pramlintide acetate, mecasermin (IGF-1), β-gluco cerebrosidase, alglucosidase-α, laronidase (α-L-iduronidase), idursuphase (iduronate-2-sulphatase), galsulphase, agalsidase-β (α-galactosidase), α-1 proteinase inhibitor, and von Willebrands factor.

The tolerogenic antigen can be derived from therapeutic antibodies and antibody-like molecules, including antibody fragments and fusion proteins with antibodies and antibody fragments. These include nonhuman antibodies, chimeric antibodies, and humanized antibodies. Immune responses to humanized antibodies have been observed in humans (Getts D R, Getts M T, McCarthy D P, Chastain E M L, & Miller S D (2010), mAbs, 2(6):682-694.). Accordingly, embodiments include a fusion molecule for tolerogenesis containing an erythrocyte-binding moiety and at least one antigen, antigenic fragment, or antigenic mimotope of one or more of these proteins, with the erythrocyte-binding moiety specifically binding, for instance, glycophorin A or a target chosen from the group consisting of Band 3, glycophorin B, glycophorin C or other members of the Erythrocyte Target Group. The erythrocyte-binding moiety may be, for instance, chosen from the group consisting of antibodies, antibody fragments, scFvs, peptide ligands and aptamers.

The tolerogenic antigen can be derived from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), and myelin basic protein (multiple sclerosis).

For example, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

The tolerogenic antigen can be one or more of the following proteins, or a fragment or peptide derived therefrom. In type 1 diabetes mellitus, several autoantigens have been identified: insulin, proinsulin, preproinsulin, glutamic acid decarboxylase-65 (GAD-65), GAD-67, insulinoma-associated protein 2 (IA-2), and insulinoma-associated protein 23 (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S1000, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, autoantigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, an autoantigen is calcium sensitive receptor. In Addison's disease, autoantigens include 21-hydroxylase, 17α-hydroxylase, and P450 side chain cleavage enzyme (P450scc); other antigens include ACTH receptor, P450c21 and P450c17. In premature ovarian failure, autoantigens include FSH receptor and α-enolase. In autoimmune hypophysitis, or pituitary autoimmune disease, autoantigens include pituitary gland-specific protein factor (PGSF) 1a and 2; another antigen is type 2 iodothyronine deiodinase. In multiple sclerosis, autoantigens include myelin basic protein, myelin oligodendrocyte glycoprotein and proteolipid protein. In rheumatoid arthritis, an autoantigen is collagen II. In immunogastritis, an autoantigen is H⁺, K⁺-ATPase. In pernicious anemia, an autoantigen is intrinsic factor. In celiac disease, autoantigens are tissue transglutaminase and gliadin. In vitiligo, an autoantigen is tyrosinase, and tyrosinase related protein 1 and 2. In myasthenia gravis, an autoantigen is acetylcholine receptor. In pemphigus vulgaris and variants, autoantigens are desmoglein 3, 1 and 4; other antigens include pemphaxin, desmocollins, plakoglobin, perplakin, desmoplakins, and acetylcholine receptor. In bullous pemphigoid, autoantigens include BP180 and BP230; other antigens include plectin and laminin 5. In dermatitis herpetiformis Duhring, autoantigens include endomysium and tissue transglutaminase. In epidermolysis bullosa acquisita, an autoantigen is collagen VII. In systemic sclerosis, autoantigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, an autoantigen is U1snRNP. In Sjogren's syndrome, the autoantigens are nuclear antigens SS-A and SS-B; other antigens include fodrin, poly(ADP-ribose) polymerase and topoisomerase. In systemic lupus erythematosus, autoantigens include nuclear proteins including SS-A, high mobility group box 1 (HMGB1), nucleosomes, histone proteins and double-stranded DNA. In Goodpasture's syndrome, autoantigens include glomerular basement membrane proteins including collagen IV. In rheumatic heart disease, an auto antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

The tolerogenic antigen can be a foreign antigen against which a patient has developed an unwanted immune response. Examples are food antigens. Embodiments include testing a patient to identify foreign antigen and creating a molecular fusion that contains the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1); from milk: α-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

### D. Immunomodulatory Agents

The particle can include one or more immunomodulatory agents, including immunosuppressant or immunostimulatory agents of regulatory T cells. Immunosuppressants are known in the art and include glucocorticoids, cytostatics (such as alkylating agents, antimetabolites, and cytotoxic antibodies), antibodies (such as those directed against T-cell receptors or 11-2 receptors), drugs acting on immunophilins (such as cyclosporine, tacrolimus, and sirolimus) and other drugs (such as interferons, opioids, TNF binding proteins, mycophenolate, and other small molecules such as fingolimod). Immunosuppressants include, but are not limited to, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin, MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliotoxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane, paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds, as well as other methods in which Rapamycin has been administered are known in the art (See, e.g. WO 95/22972, WO 95/16691, WO 95/04738, U.S. Pat. Nos. 6,015,809; 5,989,591; 5,567,709; 5,559,112; 5,530,006; 5,484,790; 5,385,908; 5,202,332; 5,162,333; 5,780,462; 5,120,727). Rapamycin analogs include, for example, everolimus, ridaforolimus, remsirolimus, umirolimus, and zotarolimus. The following are agents that may be used in combinations with antigen and immunosuppressant such as rapamycin, alone or in combination with antigen without immunosuppressant for immunomodulation. In one embodiment, the immunosuppressant is a TNF-α blocker. In another embodiment, the immunosuppressant increases the amount of adenosine in the serum, see, for example, WO 08/147482.

The compositions can be used in combination or succession with compounds that increase Treg activity or production. Exemplary Treg enhancing agents include, but are not limited to, glucocorticoid fluticasone, salmeterol, antibodies to IL-12, IFN-γ, and IL-4; vitamin D3, and dexamethasone, and combinations thereof. The compounds can increase or promote the activity of Tregs, increase the production of cytokines such as IL-10 from Tregs, increase the differentiation of Tregs, increase the number of Tregs, or increase the survival of Tregs. See also U.S. Published Application No. 2012/0276095.

Antibodies, small molecules and other compounds that reduce the bioactivity of proinflammatory cytokines can also be used. In some embodiments, the compounds reduce the bioactivity of IL-1, IL-6, IL-8, TNF-α (tumor necrosis factor alpha), TNF-β (lymphotoxin α, LT) or a combination thereof.

Another major category within biologics is tumor necrosis factor (TNF) blockers, which counteract high levels of inflammatory proteins. Etanercept (Enbrel), infliximab (Remicade) and adalimumab (Humira) are the most widely used. Another promising group is interleukin-1 (IL-1) blockers like anakinra (Kineret).

In some embodiments, the agent is an anti-inflammatory cytokine or chemokine, for example, transforming growth factor-beta (TGF-beta), interleukin (IL)-1 receptor antagonist, IL-4, IL-6, IL-10, IL-11, and IL-13. Specific cytokine receptors for IL-1, tumor necrosis factor-alpha, and IL-18 also function as pro-inflammatory cytokine inhibitors. The nature of anti-inflammatory cytokines and soluble cytokine receptors are known in the art and discussed in Opal and DePalo, Chest, 117(4):1162-72 (2000).

Retinoic acid is an additional therapeutic compound that can be used as an antinflammatory agent. See, for example, Capurso, et al., Self/Nonself, 1:4, 335-340 (2010).

Mycophenolate mofetil (MMF) and its active metabolite mycophenolic acid (MPA) are both very effective immunosuppressive agents. MMF has been used to treat autoimmune and inflammatory skin diseases. Lipsky, Lancet, 348:L1357-1359 (1996) and has become a valuable therapeutic option in children with autoimmune disease. Filler, et al., Pediatric Rheumatol., 8:1 (2010). Mycophenolic acid (MPA) is a relatively new adjuvant drug that selectively inhibits T and B lymphocyte proliferation by suppressing de novo purine synthesis. Other steroid sparing immunosuppressive agents include azathioprine, methotrexate and cyclophosphamide.

MPA is the active form of mycophenolate mofetil, which is currently used as an immunosuppressant in humans for lupus and other autoimmune disease therapy (Ginzler, et al., N Engl J Med, 353(21):2219-28 (2005)). MPA has broad immunosuppressive effects on several immune cell types. MPA blocks the de novo synthesis pathway of guanine nucleotides. T and B cell proliferation is acutely impaired by MPA because these cells lack the biosynthetic salvage pathways that could circumvent impaired de novo guanine production (Jonsson, et al., Clin Exp Immunol, 124(3): 486-91 (2001); Quemeneur, et al., J Immunol, 169(5):2747-55 (2002); Jonsson, et al., Int Immunopharmacol, 3(1):31-7 (2003); and Karnell, et al., J Immunol, 187(7): 3603-12 (2011). Furthermore, MPA can impair the activation of dendritic cells and their ability to stimulate alloantigen responses (Mehling, et al., J Immunol, 165(5):2374-81 (2000); Lagaraine, et al., Int Immunol, 17(4):351-63 (2005); and Wadia, et al., Hum Immunol, 70(9):692-700 (2009)), and promote the development of tolerogenic dendritic cells (Lagaraine, et al., J Leukoc Biol, 84(4):1057-64 (2008)). Like many immunosuppressant drugs, MPA is very hydrophobic, with a reported partition coefficient (log P value) of 3.88 (Elbarbry, et al., J Chromatogr B Analyt Technol Biomed Life Sci, 859(2): 276-81(2007)).

An immunosuppressant can be any small molecule that suppresses the function of the immune system or that increases susceptibility to infectious diseases. In certain embodiments, the immunosuppressant is an inhibitor of T cell proliferation, an inhibitor of B cell proliferation, or an inhibitor of T cell and B cell proliferation. In certain embodiments the T cell or B cell proliferation inhibitors inhibit or regulate the synthesis of guanine monophosphate. For example, the immunosuppressant can be mycophenolic acid.

Alternatively, the immunosuppressant is a prodrug of mycophenolic acid including, but not limited to, mycophenolate mofetil (marketed under the trade names CELLCEPT® by the Swedish company F. Hoffmann-La Roche Ltd.

A salt of the immunosuppressant may also be used, for example, a salt of mycophenolic acid includes, but is not limited to, the mycophenolate sodium (marketed under the trade name MYFORTIC® by Novartis. In some embodiments, the immunosuppressant is a purine analogue including, but not limited to, azathioprine (marketed under a variety of trade names including AZASAN® by Salix and IMURAN® by GlaxoSmithKline) or mercaptopurine (marketed under the trade name PURINETHOL® ((Mercaptopurine). In some embodiments the immunosuppressant is an antimetabolite that inhibits the use and/or the synthesis of purines, such as a purine nucleoside phosphorylase inhibitor.

Additionally, or alternatively, anti-inflammatory agents can be used. The anti-inflammatory agent can be non-steroidal, steroidal, or a combination thereof. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The more popular corticosteroids include prednisolone, hydrocortisone, methylprednisolone, dexamethasone, cortisone, triamcinolone, and betamethasone.

### D. Targeting Moiety

In some embodiments, one or more targeting moieties (also referred to herein as targeting molecules, and targeting signals) can be loaded into, attached to the surface of, and/or enclosed within the particle. These are not typically required. Exemplary target molecules include proteins, peptides, nucleic acids, lipids, saccharides, or polysaccharides that bind to one or more targets associated with a tissue, cell, or extracellular matrix of the liver. Preferably, the targeting moiety is displayed on and preferably conjugated to the exterior surface of the particle. Preferably, the targeting moiety increases or enhances targeting of the particles to the liver, or tissue or cells thereof including liver cells and endothelial cells.

Various techniques can be used to engineer the surface of particles, such as covalent linkage of molecules (ligands) to nanosystems (polymers or lipids) (Tosi, et al., SfN Neurosci San Diego (USA), 1:84 (2010)).

The degree of specificity with which the particles are targeted can be modulated through the selection of a targeting molecule with the appropriate affinity and specificity.

For example, antibodies are very specific. These can be polyclonal, monoclonal, fragments, recombinant, or single chain, many of which are commercially available or readily obtained using standard techniques. The targeting molecules may be conjugated to the terminus of one or more PEG chains present on the surface of the particle.

In some embodiments, the targeting moiety is an antibody or antigen binding fragment thereof that specifically recognizes a liver cell or tissue marker. Fragments are preferred since antibodies are very large, and can have limited diffusion through tissue. Suitable targeting molecules that can be used to direct the particle to cells and tissues of interest, as well as methods of conjugating target molecules to nanoparticles, are known in the art.

A particularly preferred target is DEC205+. DEC205+a cell receptor with a m.w. of 205 kDa (DEC205) (Ring, et al., J. Immuno., doi:10.4049/jimmunol.1202592 (11 pages) (2013)). It is expressed by epithelial call and dendritic cells (DCs) and facilitates antigen presentation. Compositions for targeting DEC205+ are known in the art and include, for example, anti-DEC205+ antibody and fragments and fusions thereof (see, e.g., Silva-Sinchez, PLoS ONE 10(4): e0124828. doi:10.1371/journal.pone.0124828; Spiering, et al., J Immunol., 194(10):4804-13 (2015). doi: 10.4049/jimmunol.1400986. Epub 2015 Apr. 10). It is believed that DEC205-targeted nanoparticles utilize DEC205-mediated endocytosis to gain entry into target cells, which reduces their capacity to activate antigen-specific CD4 T cells. DCs that take up antigen via DEC205 are known to cross present via MHC class I, which can promote CD8 T cell deletional tolerance in mouse models of autoimmune diabetes and EAE.

In some embodiments, density of the targeting ligand is modulated to tune the tolerance inducing effect of the carrier.

### E. Pharmaceutical Compositions

### Dosage Units

The nanoparticles can be formulated in liquid or solid form, for oral administration as a single or multiple dosage unit

The compositions are typically formulated in dosage unit form for ease of administration and uniformity of dosage. It will be understood, however, that the total daily usage of the compositions will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and other factors well known in the medical arts.

In certain embodiments, dosage units contain PBA nanoparticles encapsulating active and/or imaging agents in total dosage amounts between about 1 microgram/kg and 5 grams/kg, based on species, route of administration, number of doses and disorder to be treated. Representative ranges include 0.001 mg/kg to about 1000 mg/kg, from about 0.01 mg/kg to about 500 mg/kg, from about 0.1 mg/kg to about 500 mg/kg, from about 0.5 mg/kg to about 500 mg/kg, from about 1 mg/kg to about 5000 mg/kg, from about 0.1 mg/kg to about 100 mg/kg, or from about 1 mg/kg to about 100 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

### Excipients

The nanoparticles can be formulated in liquid or solid form, for oral administration as a single or multiple dosage unit.

The effective dosage may be dependent on the concentration of excipients and how they are added. TGR5 activation results in anti-inflammatory immunity, anti-fibrotic activity, induction and secretion of GLP-1 from enteroendocrine L cells together with increased energy expenditure in adipose tissue32. pUDCA may not only significantly lower the dose but amplify the range of UDCA function because its monomeric counterpart, UDCA, is an intrinsically weak TGR5 agonist.

The compositions are typically formulated in dosage unit form for ease of administration and uniformity of dosage. It will be understood, however, that the total daily usage of the compositions will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and other factors well known in the medical arts.

Excipients and/or carriers may be chosen based on the dosage form to be administered, the active agents being delivered, etc. Suitable excipients include surfactants, emulsifiers, emulsion stabilizers, anti-oxidants, emollients, humectants, chelating agents, suspending agents, thickening agents, occlusive agents, preservatives, stabilizing agents, pH modifying agents, solubilizing agents, solvents, flavoring agents, colorants, and other excipients. As used herein, “excipient” does not include any bile acid or polymer thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

Suitable surfactants include, but are not limited to, anionic surfactants, non-ionic surfactants, cationic surfactants, and amphoteric surfactants.

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

Suitable chelating agents include, but are not limited to, EDTA, and combinations thereof.

Suitable humectants include, but are not limited to, glycerin, butylene glycol, propylene glycol, sorbitol, triacetin, and combinations thereof.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Excipients may include suspending agents such as sterile water, phosphate buffered saline, saline, or a non-aqueous solution such as glycerol.

Particles can be provided as dry powders following spray drying or lyophilization.

Particles may be compressed into tablets, which may in turn be coated with a material such as an EUDRAGIT® to prevent release of the particles after passage through the stomach.

Particles may also be encapsulated in hard or soft gels, such as gelatin and alginate capsules and the enteric formulated soft gels sold by Banner Pharmaceuticals.

Particles may also be formulated for administration to mucosal surfaces, such as the mouth, nasal cavity, oral cavity, pulmonary system, rectal or vaginal surfaces.

Particles may also be provided in a kit, where the material to be delivery is provided separately from the dosage unit, then combined in powder or dry form or in solution prior to use. The agent to be delivered can be entrapped, encapsulated or bound to the bile salt polymers chemically or physically.

### III. Methods of Making Nanoparticles

The pUDCA nanoparticles described herein can be prepared by a variety of methods.

The following are representative methods.

### A. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid nanoparticles containing core material.

The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a nanoparticles composed of polymer or copolymer shell with a core of liquid material.

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid particles containing core material.

### B. Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

### C. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

### D. Coacervation

Encapsulation procedures for various substances using coacervation techniques have been described in the prior art, for example, in GB-B-929 406; GB-B-929 401; U.S. Pat. Nos. 3,266,987; 4,794,000; and 4,460,563. Coacervation is a process involving separation of colloidal solutions into two or more immiscible liquid layers (Dowben, R. General Physiology, Harper & Row, New York, 1969, pp. 142-143.). Through the process of coacervation compositions contained of two or more phases and known as coacervates may be produced. The ingredients that contain the two phase coacervate system are present in both phases; however, the colloid rich phase has a greater concentration of the components than the colloid poor phase.

### E. Spray Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 NI/hr, and nozzle diameter=0.5 mm. Microparticles ranging between 1-10 microns are obtained with a morphology which depends on the type of polymer used.

### F. Fluorine-Mediated Supramolecular Assemblies

Fluorinated bile acid units (either linear or branched) can be synthesized by reaction of a terminal carboxylate or hydroxyl group with an alkylfluorate anhydride (AFAA). The product can extracted into water initiating a fluorophobic effect, in which spontaneous aggregation of the fluorinated building blocks takes place preferentially and differently from a hydrophobic effect. Such assembly is dependent on both the thermal energy, extent of fluorination, enabling some thermodynamic and kinetic control over the final morphology. Fluorophobic-mediated self-assembly will provide the cohesive forces for aggregation and may serve as an intrinsically imageable system through 19F NMR. Fluorinated bile acids will also have a distinctly different biodistribution and clearance time which may serve to enhance the residence time of the system in the GI tract or in the pancreatic regions.

### IV. Methods of Use

### A. Routes of Administration

The particles are preferably administered orally, and show enhanced uptake by target organ such as the pancreas, liver, or colon. Oral administration can be achieved via oral gavage, or by swallowing of the composition in liquid or solid form. The liquid forms of orally administered compositions can be in a form of a solution or a liquid gel. Solid forms of orally administered compositions can be in the form of capsules, soft and hard gels, tablets, pills, powders, and granules.

Although described with reference to oral administration, it is understood that the same delivery may be achieved by delivery to a mucosal surface such as the mouth, nasal cavity, lung, lung, rectum or vagina, or delivery through intravenous (i.v.) injection.

The desired dosage may be delivered orally once a day, or multiple times a day. For example, the desired dosage may be delivered orally three times a day, two times a day, once a day, every other day, every third day, every week, every two weeks, every three weeks, or every four weeks. In certain embodiments, the desired dosage may be delivered using multiple daily administrations (e.g., two, three, four, five, six, seven, eight, nine, ten, eleven, twelve, thirteen, fourteen, or more administrations).

### B. Disorders to be Treated

A method of preventing, suppressing or treating a disease or condition may include administering to a subject in need thereof an oral dosage unit of the pharmaceutical composition containing the untargeted PBA nanoparticles encapsulating the one or more agent(s); delivering an effective amount of one or more agent(s), optionally to targeted tissue such as pancreas, liver, or colon; wherein the agent is released from the PBA nanoparticles at the target tissues, resulting in prevention, suppression or treatment of the disease.

The formulations are particularly useful for treatment of neoplasma of the colon, liver, spleen, pancreas, or adjacent areas. The formulations are also very useful in treating diseases of the gastrointestinal tract, including ulcers, irritable bowel disease (IBD), and colon cancers. The formulations are useful in treatment of inflammatory diseases and autoimmune and allergenic disease. The formulations are also efficacious in treating diseases such as diabetes.

### 1. Autoimmune and Inflammatory Diseases and Conditions

It will be appreciated that the compositions and methods disclosed herein have a broad range of applications including, but not limited to, treatment of autoimmune disease, therapies for transplant rejection, adjuvants for enhancement of immunosuppressive function, and cell therapies involving Tregs or tolerogenic DCs.

In some embodiments, the compositions and methods are used to treat chronic and persistent inflammation, which can be a major cause of the pathogenesis and progression of an autoimmune diseases or inflammatory condition. Accordingly, methods of treating inflammatory and autoimmune diseases and disorders can include administering to a subject in need thereof, an effective amount of a particle formulation or a pharmaceutical composition thereof, to reduce or ameliorate one or more symptoms of the disease or condition. Some of the applications are discussed in more detail below.

Representative inflammatory or autoimmune diseases and disorders that may be treated using the disclosed compositions and methods include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), IgA nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

### 2. Inhibition of Epitope Spreading

Epitope spreading refers to the ability of B and T cell immune response to diversify both at the level of specificity, from a single determinant to many sites on an auto antigen, and at the level of V gene usage (Monneaux, F. et al., Arthritis & Rheumatism, 46(6): 1430-1438 (2002). Epitope spreading is not restricted to systemic autoimmune disease. It has been described in T cell dependent organ specific diseases such as IDDM and multiple sclerosis in humans and EAE induced experimental animals with a variety of myelin proteins.

Epitope spreading involves the acquired recognition of new epitopes in the same self molecule as well as epitopes residing in proteins that are associated in the same macromolecular complex. Epitope spreading can be assessed by measuring delayed-type hypersensitivity (DTH) responses, methods of which are known in the art.

Therefore, in some embodiments, a method for inhibiting or reducing epitope spreading in a subject includes administering to the subject an effective amount of nanocarrier. In a preferred embodiment the particle formulation inhibits epitope spreading in individuals with multiple sclerosis.

### 3. Allergies

A similar methodology can be used to treat allergies, substituting the allergen of interest for the autoimmune stimulus. Typically, particles are administered to a subject in an effective amount to reduce or inhibit an allergy or allergic reaction.

Allergies are abnormal reactions of the immune system that occur in response to otherwise harmless substances. Allergies are among the most common of medical disorders. It is estimated that 60 million Americans, or more than one in every five people, suffer from some form of allergy, with similar proportions throughout much of the rest of the world. Allergy is the single largest reason for school absence and is a major source of lost productivity in the workplace.

An allergy is a type of immune reaction. Normally, the immune system responds to foreign microorganisms or particles by producing specific proteins called antibodies. These antibodies are capable of binding to identifying molecules, or antigens, on the foreign particle. This reaction between antibody and antigen sets off a series of chemical reactions designed to protect the body from infection. Sometimes, this same series of reactions is triggered by harmless, everyday substances such as pollen, dust, and animal danders. When this occurs, an allergy develops against the offending substance (an allergen.)

Mast cells, one of the major players in allergic reactions, capture and display a particular type of antibody, called immunoglobulin type E (IgE) that binds to allergens. Inside mast cells are small chemical-filled packets called granules. Granules contain a variety of potent chemicals, including histamine.

Immunologists separate allergic reactions into two main types: immediate hypersensitivity reactions, which are predominantly mast cell-mediated and occur within minutes of contact with allergen; and delayed hypersensitivity reactions, mediated by T cells (a type of white blood cells) and occurring hours to days after exposure.

Inhaled or ingested allergens usually cause immediate hypersensitivity reactions. Allergens bind to IgE antibodies on the surface of mast cells, which spill the contents of their granules out onto neighboring cells, including blood vessels and nerve cells. Histamine binds to the surfaces of these other cells through special proteins called histamine receptors. Interaction of histamine with receptors on blood vessels causes increased leakiness, leading to the fluid collection, swelling and increased redness. Histamine also stimulates pain receptors, making tissue more sensitive and irritable. Symptoms last from one to several hours following contact. In the upper airways and eyes, immediate hyper-sensitivity reactions cause the runny nose and itchy, bloodshot eyes typical of allergic rhinitis. In the gastrointestinal tract, these reactions lead to swelling and irritation of the intestinal lining, which causes the cramping and diarrhea typical of food allergy. Allergens that enter the circulation may cause hives, angioedema, anaphylaxis, or atopic dermatitis.

Allergens on the skin usually cause delayed hypersensitivity reaction. Roving T cells contact the allergen, setting in motion a more prolonged immune response. This type of allergic response may develop over several days following contact with the allergen, and symptoms may persist for a week or more.

Allergens enter the body through four main routes: the airways, the skin, the gastrointestinal tract, and the circulatory system. Airborne allergens cause the sneezing, runny nose, and itchy, bloodshot eyes of hay fever (allergic rhinitis). Airborne allergens can also affect the lining of the lungs, causing asthma, or conjunctivitis (pink eye). Exposure to cockroach allergens has been associated with the development of asthma. Airborne allergens from household pets are another common source of environmental exposure. Allergens in food can cause itching and swelling of the lips and throat, cramps, and diarrhea. When absorbed into the bloodstream, they may cause hives (urticaria) or more severe reactions involving recurrent, non-inflammatory swelling of the skin, mucous membranes, organs, and brain (angioedema). Some food allergens may cause anaphylaxis, a potentially life-threatening condition marked by tissue swelling, airway constriction, and drop in blood pressure. Allergies to foods such as cow's milk, eggs, nuts, fish, and legumes (peanuts and soybeans) are common. Allergies to fruits and vegetables may also occur. In contact with the skin, allergens can cause reddening, itching, and blistering, called contact dermatitis. Skin reactions can also occur from allergens introduced through the airways or gastrointestinal tract. This type of reaction is known as atopic dermatitis. Dermatitis may arise from an allergic Dermatitis may arise from an allergic response (such as from poison ivy), or exposure to an irritant causing nonimmune damage to skin cells (such as soap, cold, and chemical agents). Injection of allergens, from insect bites and stings or drug administration, can introduce allergens directly into the circulation, where they may cause system-wide responses (including anaphylaxis), as well as the local ones of swelling and irritation at the injection site.

These can be treated by administration of anti-inflammatories, or by inducing tolerance to the antigen, as discussed in more detail below.

### 4. Diabetes

Diabetes, or diabetes mellitus, is due to either the pancreas not producing enough insulin or the cells of the body not responding properly to the insulin produced. There are three main types of diabetes mellitus:
Type 1 Diabetes results from the pancreas' failure to produce enough insulin or active insulin; this form was previously referred to as “insulin-dependent diabetes mellitus” (IDDM) or “juvenile diabetes”,
Type 2 Diabetes begins with insulin resistance, a condition in which cells fail to respond to insulin properly. As the disease progresses a lack of insulin may also develop; this form was previously referred to as “non insulin-dependent diabetes mellitus” (NIDDM) or “adult-onset diabetes”; and
Gestational diabetes, the third main form, occurs when pregnant women, without a previous history of diabetes, develop a high blood sugar level.

Type 1 diabetes must be managed with insulin injections. Type 2 diabetes may be treated with medications with or without insulin. Gestational diabetes usually resolves after the birth of the baby.

People with type 1 diabetes need insulin therapy to survive. Many people with type 2 diabetes or gestational diabetes also need insulin therapy. Medications used for treating T2D include over 20 types of injectable insulin, and orally administered drugs such as meglitinides, sulfonylureas, metformin, canagliflozin, dapagliflozin, thiazolidinediones, pioglitazone, rosiglitazone, acarbose, pramlintide, exenatide, liraglutide, long-acting exenatide, albiglutide, dulaglutide, and dipeptidyl peptidase-4 (DPP-IV) inhibitors (sitagliptin, saxagliptin, linagliptin). These agents are collectively referred to as “anti-diabetics”.

The compositions can be used to treat the inflammation of the pancreas (pancreatitis), the liver (hepatitis), or the colon (IBD). The PBA nanoparticles encapsulating a therapeutic and/or imaging agent, can pass through the fenestrated vasculature of an inflamed tissue, and are retained longer within the inflamed tissue, due to their size, compared to biologics or small molecule drugs (1-10 nm). They are also effectively internalized by antigen-presenting cells (such as macrophages and dendritic cells), making the PBA nanoparticles suitable for agent delivery to inflamed tissues and the cells of the immune system.

Two forms of pancreatitis, acute and chronic pancreatitis, can be treated with oral administration of the PBA compositions.

Acute pancreatitis is a sudden inflammation that lasts for a short time. It may range from mild discomfort to a severe, life-threatening illness. In severe cases, acute pancreatitis can result in bleeding into the gland, serious tissue damage, infection, and cyst formation. Severe pancreatitis can also harm other vital organs such as the heart, lungs, and kidneys.

Chronic pancreatitis is long-lasting inflammation of the pancreas. It most often happens after an episode of acute pancreatitis. Heavy alcohol drinking is another big cause. Damage to the pancreas from heavy alcohol use may not cause symptoms for many years, but then the subject may suddenly develop severe pancreatitis symptoms. Subjects with acute pancreatitis are treated with IV fluids and pain medications in the hospital. Chronic pancreatitis can be difficult to treat. It involves pain relief and improved nutrition. Subjects are generally given pancreatic enzymes or insulin.

The inflammation of the liver (hepatitis) is characterized by the presence of inflammatory cells in the tissue of the organ. Hepatitis may occur with limited or no symptoms, but often leads to jaundice (a yellow discoloration of the skin, mucous membrane, and conjunctiva), poor appetite, and malaise. Hepatitis is acute when it lasts less than six months and chronic when it persists longer.

Acute hepatitis can be self-limiting (healing on its own), can progress to chronic hepatitis, or, rarely, can cause acute liver failure. Chronic hepatitis may have no symptoms, or may progress over time to fibrosis (scarring of the liver) and cirrhosis (chronic liver failure). Cirrhosis of the liver increases the risk of developing hepatocellular carcinoma.

Viral hepatitis is the most common cause of liver inflammation. Other causes include autoimmune diseases and ingestion of toxic substances (notably alcohol), certain medications (such as paracetamol), some industrial organic solvents, and plants. Antiretroviral drugs such as tenofovir and entecavir are used for the treatment of chronic hepatitis B.

### 5. Inflammatory Bowel Disease.

Inflammatory bowel disease (IBD) is a broad term that describes conditions with chronic or recurring immune response and inflammation of the gastrointestinal tract. The two most common inflammatory bowel diseases are ulcerative colitis and Crohn's disease. Inflammation affects the entire digestive tract in Crohn's disease and only the large intestine in ulcerative colitis. Both illnesses are characterized by an abnormal response to the body's immune system.

Crohn's disease is treated with medications designed to suppress the immune system's abnormal inflammatory response that causes the symptoms. Suppressing inflammation offers relief from common symptoms like fever, diarrhea, and pain, and healing of the intestinal tissues. Combination therapy could include the addition of a biologic to an immunomodulator. As with all therapies, there are risks and benefits of combination therapies. Combining medications with immunomodulatory therapies can increase the effectiveness of IBD treatment.

Examples of agents used to treat IBD symptoms include, but are not limited to, sulfasalazine, mesalamine, olsalazine, and balsalazide that contain 5-aminosalicylate acid (5-ASA), corticosteroids, immunomodulators, antibiotics, and biologic therapies.

### 6. Delivery of Antigen and Induction of Tolerance

Methods of inducing tolerance are provided. The methods are generally based on the principle that immune suppressive drug and/or antigen can be targeted to the liver using the disclosed particles and will be taken up by liver dendritic cells (DC) and/or liver endothelial cells (EC). The liver is an organ of interest for targeting agents for induction of tolerance against those agents. It is believed that compositions loaded with antigen of interest and/or in combination with an immunosuppressive agent, will facilitate peripheral tolerance against the antigen of interest. The targeting can be passive (i.e., retention in the liver) or active (i.e., targeted to specific cells in the liver). Accordingly, a liver targeting moiety is optional.

Particles carrying antigen and/or immunosuppressive drug are preferably spatially localized to the same liver dendritic cell or liver endothelial cell for initiation of tolerance. Therefore, although different particles carrying antigen in one set and immunosuppressive agent in another set and injected together are contemplated, nanoparticles carrying both agents and targeted to liver dendritic cells or endothelial cells are preferred.

A preferred strategy generally includes administration of particles including an antigen and immunosuppressive agent that are retained in the liver and taken up by liver antigen presenting cell or endothelial cells. Tolerogenic dendritic cells then circulate throughout the body to induce tolerance (peripheral tolerance) to the encapsulated antigen. Exemplary cells that can serve as live antigen presenting cells include liver dendritic cells (DCs), liver endothelial cells, Kupffer cells, Hepatic stellate cells, hepatocytes, and other cells that present antigens to the liver.

Liver DCs or ECs drain to local lymph nodes (Celiac). They acquire a tolerogenic program that induces the expansion of antigen-specific regulatory T cells (Tregs). APCs can also present antigen to T cells in the sinusoids without migrating out. Furthermore, the antigen may be processed by the DC while it is in the liver or the lymph nodes, or even while migrating between them. Generally, intracellular accumulation, trafficking or retention of the carrier in liver cells is important for tolerance induction.

Antigen-presenting cells also express anti-inflammatory markers or markers signifying the initiation of a tolerogenic phenotype. Tregs migrate from the lymph nodes into circulation and induce system-wide tolerance.

A preferred strategy can be summarized in five steps:
1) Homing to liver;
2) Uptake by dendritic cells and/or APCs in the liver;
3) Drainage to local lymphatics;
4) Expansion of regulatory T cells;
5) Migration into the bloodstream and initiation of peripheral tolerance.

The methods disclosed herein generally include administering a subject in need thereof an effective amount of the disclosed particles, most typically in a pharmaceutical composition, to induce or increase tolerance to an antigen of interest. In particular embodiments, the composition increases the number or activity of regulatory T cells. Accordingly, pharmaceutical compositions including particles including a tolerogenic antigen and/or an immunosuppressive agent present in the composition in an effective amount to induce liver dendritic cells and/or liver endothelial cells to acquire a tolerogenic phenotype, induce the expansion of antigen-specific regulatory T cells (Tregs), or a combination thereof, and method of use thereof are provided.

Robust tolerance may be achieved through induction of antigen-specific Tregs, polyclonal Tregs, Tr1 cells, other CD4 cells expressing PD-L1 or CTLA-4, CD8 cell deletion/anergy, even Bregs. Thus, in some embodiments, the composition is administered in an effective amount to acquire a tolerogenic program that reduces or prevents immunogenicity against a desired antigen, for example, the antigen delivered by the particle.

Administration is not limited to the treatment of an existing condition or disease but can also be used to prevent or lower the risk of developing such diseases in an individual, i.e., for prophylactic use. The compositions can be utilized in prophylactic vaccines or therapies, or therapeutic vaccines or therapies, which can be used to initiate or enhance a subject's immune tolerance to a pre-existing antigen, or to a new antigen.

The desired outcome of a prophylactic, therapeutic or de-sensitized immune response may vary according to the disease, according to principles well known in the art. Similarly, immune tolerance may completely treat a disease, may alleviate symptoms, or may be one facet in an overall therapeutic intervention against a disease.

Potential candidates for prophylactic vaccination include individuals with a high risk of developing autoimmunity against a certain self-antigen, and patients receiving recombinant protein therapy (FVIII or FIX).

### C. Imaging

In other embodiments, the methods of using the pharmaceutical compositions may include methods of non-invasively imaging the target organ as a whole, or distinct microenvironments within the target organ, such as pockets of inflammation, leaky vasculature, or neoplasms. In these embodiments, the methods include administering to a subject in need thereof an oral dosage unit of the pharmaceutical composition containing the untargeted PBA nanoparticles encapsulating an effective amount of an imaging agent; delivering the effective amount of the imaging agent to target tissue, such as pancreas, liver, or colon; optionally releasing the effective amount of the imaging agent from the nanoparticles at the target tissues; which results in enhanced detection of target tissue, or a distinct microenvironment within the target tissue, via non-invasive imaging.

Imaging modalities suitable for detecting the PBA nanoparticles, and/or the agents therein include positron-emission tomography (PET), computed tomography (CT), magnetic resonance imaging (MRI), ultrasound imaging (US), and optical imaging. Suitable imaging agents (tracers) include radionuclide-labeled small molecules, such as F-18 fluorodeoxyglucose, superparamagnetic iron oxide (SPIO), gadolinium, europium, diethylene triamine pentacetic acid (DTPA), 1,4,7,10-tetraazacyclododecane-1,4,7,10-tetraacetic acid (DOTA) and their derivatives, gas, and fluorescent tracers. Such suitable modalities with respective tracers are known in the art (Baum et al., Theranostics, 2(5)437-447 (2012)).

### D. Combined Therapy and Diagnosis

In other embodiments the methods of preventing, suppressing or treating a disease or condition, and methods of non-invasively imaging the target organ or tissue, are combined. In this embodiment, the pharmaceutical compositions contain untargeted PBA nanoparticles encapsulating both a therapeutic and a diagnostic/imaging agent. The method may include administering to a subject in need of prevention, suppression, or treatment of disease in and imaging of a target tissue an oral dosage unit of the pharmaceutical composition containing the untargeted PBA nanoparticles encapsulating an effective amount of one or more active agent(s) and an effective amount of an imaging agent; delivering the PBA nanoparticles to target tissue, such as pancreas, liver, or colon; releasing the effective amount of the one or more agent(s) and, optionally, the effective amount of the imaging agent, from the PBA nanoparticles at the target tissues, resulting in prevention, suppression or treatment of the disease, and enhanced detection of target tissue, or a distinct microenvironment within the target tissue, via non-invasive imaging.

The present invention will be further understood by reference to the following non-limiting examples.

The Examples show that pUDCA works through parallel mechanisms involving protective transport, enhancement in recognition, metabolic and anti-inflammatory immune signals. The formulation of pUDCA NP began with the monomer UDCA, well known for its established medicinal benefits, followed by polymerization and then formulation into NP. The polymerization and formulation steps expanded the benefits beyond what can be achieved with monomer alone or even monomer on the surface of particles, as validated in the mechanistic studies that follow. The efficacy of pUDCA is due to multiple mechanisms. The first is protective transport facilitating improvements in pharmacokinetics and biodistribution of encapsulated agents.

The present invention will be further understood by reference to the following non-limiting examples.

### EXAMPLES

### Example 1: Polymeric BAs not Only Facilitate the Formulation of Orally Ingestible Therapeutic Nanoparticles but Also Provide a Broad-Spectrum of Bioactivity

There are two reasons the nanoparticle provide a broad-spectrum of activity:
1) they can be protective in nature, and increase intestinal permeation and thus the systemic bioavailability of associated agents; and
2) they possess signaling functions that can regulate glucose metabolism and immunity through binding of BA receptors and thus function as effector therapeutic systems.

The rationale for polymerization was based on the notions that: 1) polymerization facilitates a strategy for encapsulation and release of a wide range of therapeutics of interest including insulin. In other words, solid, stable, biodegradable polymeric carriers in contrast to monomeric BA micelles, which are inherently unstable. 2) Fabrication of such polymeric NPs enable sustained release of encapsulated agents if the polymers are degradable in aqueous environments. 3) Polymeric BA systems as robust carriers present BA differently (in close proximity and higher density) than, say, BA monomers hybridized on the surface of another type of polymer NPs. Furthermore, if the BA monomer has intrinsic therapeutic effect, then this effector function is be amplified with polymerization and its bioavailability is longer lasting in contrast to BA monomers on particles which may be easily released from the surface after oral ingestion. Furthermore, the sustained availability of BA over the drug release period may be a desired element for combinatorial more than additive activity. 4) The pH stimulus response of BAs which is due to their ionization potential and protonation at low pH offers stomach protection while enhancing BA with multivalency for binding to its receptors. This multivalent response not only amplifies the degree of ionization, but also kinetically amplifies the low pH protection and higher pH deprotection response time as particles transit from the stomach to the intestinal milieu. 5) Polymeric multivalency results in high binding avidity to BA receptors which results in conversion of a weak BA agonist into a stronger form upon polymerization. Stronger agonists enable greater receptor activation and therapeutic signaling functions at lower doses.

UDCA has an established record of use for lowering insulin resistance in Type 2 Diabetes (T2D), however this usage is dose-dense (typically 40-450 mg/kg in mice and for 2-20 weeks orally). UDCA is rarely tested in T1D since it mainly impacts insulin sensitivity. The functional impact of pUDCA extends beyond improvements in transport of encapsulated agent (such as insulin) in addition to amplification of its effector function beyond what the monomer can achieve on its own. UDCA can trigger protein kinase cascade cell activation, regulate glucose, and energy homeostasis if it stably binds to extracellular Takeda G-protein coupled receptors (TGR5), and can regulate nuclear factor κB (NF-κB) and signal kinases such as protein kinase B (Akt). TGR5 activation also results in anti-inflammatory immunity, anti-fibrotic activity, induction and secretion of GLP-1 from enteroendocrine L cells together with increased energy expenditure in adipose tissue. pUDCA may not only significantly lower the dose but amplify the range of UDCA function because its monomeric counterpart, UDCA, is an intrinsically weak TGR5 agonist.

From the standpoint of improvements in insulin transport, biodistribution and pharmacokinetics, BAs are natural emulsifiers. Thus, biodegradable, polymeric BA would be even better in solubilization of lipids and fats in the body. Generally, BAs function as digestive aid through their self-assembly with lipids into micelles; enabling better molecular biodistribution and blood circulation of orally ingested fatty substances. Bile and pancreatic digestive juices are known to secrete into the duodenum and bile, specifically, is shuttled from the ileum back to the liver through portal circulation then once again returned back to the intestines for further digestion of ingested fatty foods. This circulatory action of BAs from the intestines to the bile duct and back is a process termed, “Enterohepatic Circulation”. Because of the enhanced binding of polymeric BA NP, biodistribution is affected and circulation lifetime is longer.

### Methods and Materials

The methods used in the Examples were as follows.

Reagents and antibodies. All bile acids, para-toluenesulfonic acid, 4dimethylaminopyridine (DMAP), poly(vinyl alcohol) (PVA), Tween 20, pepsin, triamterene, lipopolysaccharide (LPS), and ovalbumin (OVA) were obtained from Sigma and Sigma-Aldrich. Cyclophosphamide (CY), anhydrous methylene chloride, anhydrous pyridine, diisopropyl carbodiimide, and anhydrous methanol were purchased from ACROS. Poly(lactic-co-glycolic acid) (PLGA, inherent viscosity 0.55-0.75 dL/g, carboxyl terminal) from Durect was used as a control polymer. Rapamycin (RAPA, LC Laboratories), mouse insulin (INS, R&D systems), 1,1′-dioctadecyl-3,3,3′,3′-tetramethylindotricarbocyanine iodide (DIR, Biocompare), and coumarin 6 (ACROS) were encapsulated in NPs. EUDRAGIT® FS 30D was obtained from Evonik and CpG was purchased from InvivoGen. Antibodies for CD8 (APC), CD44 (PE), CD4 (APC), CD25 (Alexa Fluor-700), CD11c (PE-Cy7), F4/80 (Alexa Fluor-647), F4/80 (Alexa Fluor-700), and CD206 (FITC) were obtained from BioLegend. Foxp3 (PE) and CD86 were purchased from Invitrogen and eBioscience, respectively. Recombinant Human GPCR TGR5 protein, Atto565-conjugated TGR5 antibody, and blocking buffer were obtained from Abcam and used for competitive binding study.

Cells. The human colon adenocarcinoma Caco-2 cells were purchased from ATCC. The cells were cultured in Dulbecco's Modified Eagle's Medium (DMEM, Life Technologies) containing 4.5 g/L glucose, 10% Fetal Bovine Serum (FBS, Atlanta Biologicals), antibiotics (100 units/mL penicillin and 100 μg/mL streptomycin, Gibco), and 1% nonessential amino acid (NEAA, Gibco). Long bones and spleens were harvested from mice (C57BL/6 or Rag2/OTII) post cervical dislocation. Bone marrow was eluted from long bones and spleens were macerated using Roswell Park Memorial Institute (RPMI)-1640 (Life Technologies) media supplemented with 10% FBS. Red blood cells (RBCs) in the sample were lysed using ammonium-chloride-potassium (ACK) lysing buffer (Lonza). Bone-marrow derived macrophages (BMMs) were cultured in Roswell Park Memorial Institute (RPMI, Life Technologies) media with macrophage colony-stimulating factor (MCSF, 10 ng/mL, Sigma-Aldrich). Bone-marrow derived dendritic cells (BMDCs) were generated using a conventional expansion protocol in which 5×10⁵ cells/mL were plated in RPMI supplemented with 20 ng/mL GM-CSF (Sigma-Aldrich) and cultured for 5 days. On day 5, non-adherent cells were collected and cultured in GM-CSF media for an additional 2 days. CD4+ T cells were purified from splenocyte population in C57BL/6 using an EasySep™ Mouse CD4+ T Cell Isolation Kit (STEMCELL Technologies). All cells were cultured at 37° C. in a humidified atmosphere of 5% CO₂.

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO). The same experiment was performed in the presence of TGR5 antagonist, triamterene (50 μg/mL) as a control to differentiate inherent insulin production from the cells without TGR5 activation and used to normalize the results. Bioactivity of released insulin from pUDCA were measured using Chinese hamster ovary cells that were transfected with the gene to express insulin receptor (CHO INSR cells, ATCC). The released insulin from pUDCA at 3 or 24 h was incubated with CHO INSR cells for 1 h and phosphorylated protein kinase B (pAkt) level was measured by ELISA (Abeam). The pAkt production from CHO cells incubated with fresh or denatured insulin was compared to calculate percent bioactivity.

Animals. C57BL/6 mice (B6, 6-8-week-old, female) were obtained from Harlan Sprague Dawley Inc. NOD mice (NOD/ShiLtJ, 8-week-old, female) and Nude mice (athymic nude, nu/nu, 7-week-old, female) were supplied by Jackson Laboratory. The mice were housed in autoclaved micro-isolator cages that were placed in a positive pressure containment rack. Ossabaw Swine (17-month-old, 42 kg) derived from the Ossabaw barrier islands were used. All experiments and maintenance were carried out according to an approved protocol from the Yale University Institutional Animal Care and Use Committee.

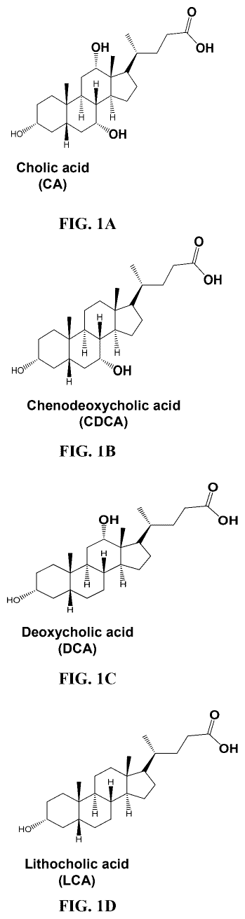

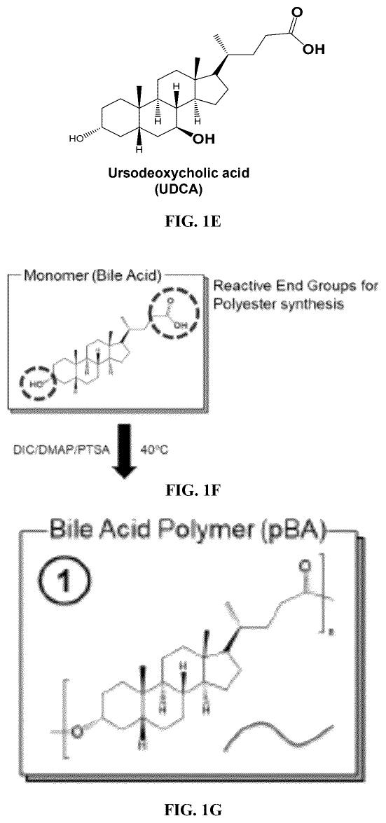

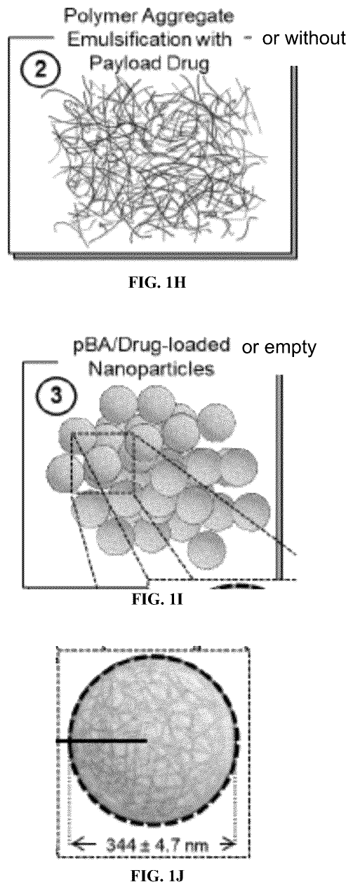

Polymer Synthesis and Nanoparticle (NP) formulation. Poly(bile acid)s (pBAs) were synthesized by esterification of the carbon 24 group (FIGS. 1F-1J) of bile acid monomers, as shown in FIGS. 1A-1E, chenodeoxycholic acid (CDCA), deoxycholic acid (DCA), lithocholic acid (LCA), and ursodeoxycholic acid (UDCA), respectively. BAs (5.4 mmol), paratoluenesulfonic acid (0.652 mmol), and DMAP (0.652 mmol) were added in 60 mL of a 5:1 anhydrous methylene chloride to anhydrous pyridine solvent mixture and stirred at 40° C. to yield a clear solution. To the reaction mixture, 6.92 mmol of diisopropyl carbodiimide was added and the reaction was allowed to proceed for 2 h in the nitrogen atmosphere. The polyester product, pBA, was precipitated into 400 mL of cold anhydrous methanol collected by centrifugation (Centrifuge 5810R, Eppendorf) and dried to retain a white powder. Polymerization was confirmed by nuclear magnetic resonance (NMR) and gel permeation chromatography (GPC). ¹H and 2D-(COSY, DQF-COSY, HSQC and HMBC) NMR spectral data for UDCA and poly(ursodeoxycholic acid) (pUDCA) were recorded on an Agilent NMR spectrometer (Agilent) at 600 MHz with a 3 mm cold probe or 400 MHz and ¹³C NMR data was measured at 100 MHz magnetic field. Chloroform-d₁ (99.96%, Cambridge Isotope Laboratories, Inc.) was used as the deuterated NMR solvent and solvent reference signals (δ_{H} 7.25, 6c 76.98) for all of the NMR experiments. The molecular weight (MW) for pBAs (10 mg/mL in chloroform) were evaluated with GPC using a Waters HPLC system equipped with a model 1515 isocratic pump, a 717 plus autosampler, and a 2414 refractive index (RI) detector with Waters Styragel columns HT6E and HT2 in series. Chloroform was utilized as the mobile phase with a flow rate of 1 mL/min and both the columns and RI detector were maintained at 40° C. MW characteristics were determined relative to a calibration curve generated from narrow polydispersity polystyrene standards (Aldrich Chemical). Empower II GPC software was used for running the GPC instrument and subsequent chromatographic analysis. pBA or PLGA or the mixture (50/50, w/w) NPs encapsulating dyes (DIR or coumarin 6), drugs (RAPA or INS) or iron oxide were formulated using an water-in oil-in water (W/O/W) double emulsion technique (FIGS. 1H-1J). Polymers or the mixture (100 mg) was dissolved in 2 mL chloroform containing DIR (1 mg), coumarin 6 (10 mg), RAPA (10 mg) or iron oxide (1 mg). Phosphate-buffered saline (PBS, 100 μL) or the PBS containing INS (10 μg) was added dropwise to the chloroform polymer solution while vortexing and homogenized using an IKA T25 Digital Ultra-Turrax (IKA). This dispersant phase was then added dropwise to a continuous phase of 5% PVA and homogenized. The mixture was then added dropwise to 200 mL of 0.2% PVA and left stirring for 2 h to evaporate the solvent. NPs were collected by centrifugation at 12,000 rpm for 20 min at 4° C. and then washed 3 times with deionized water. The particles were lyophilized and stored at −20° C. The hydrodynamic diameter and surface charge of NPs were measured by a Malvern Zetasizer. A dispersion of NPs was filtrated through a 0.45 m Millipore filter into cuvettes prior to the measurements. Dynamic light scattering was measured by back-scattering at a detection angle of 1730 at the wavelength of 532 nm and the hydrodynamic radius was calculated using the Stokes-Einstein equation. The morphology of the NPs was observed by Hitachi S-4800 High Resolution scanning electron microscopy (SEM, Norcross). A dispersion of NPs in ethanol (2 L) was placed on the wafer substrate and dried at room temperature. The sample was mounted on the aluminum sample holder and then gold sputtered. The NPs were observed with an accelerating voltage of 15 kV at a working distance of 4 mm. Release of DIR and insulin was measured in the stomach-mimicking media. NPs were dispersed in the media (citrate buffer solution, pH 2.0) at 37° C. in the presence of pepsin (10 mg/mL). At each time point, NPs were centrifuged, and supernatant was collected to measure the amount of DIR released from the particles using a plate reader (λₑₓ 750 nm, λₑₘ 790 nm, SpectraMax M5, Molecular Devices). Insulin release was quantified by the BCA assay. EUDRAGIT® coated PLGA (PLGA® EUDRAGIT®) was prepared by dispersing PLGA in 5 wt % EUDRAGIT® solution and centrifugation.

Permeability of NPs though human intestinal epithelial cell layer. Caco-2 cells seeded at 7×10⁴ cells/cm² on 0.4 m pore transwell filters (Corning). Cells were grown to confluence and allowed to mature for approximately 30 days at 37° C. and 5% CO₂. Prior to performing permeability studies, the transepithelial electrical resistance (TEER) was measured using an epithelial voltohmmeter (EVOM™ Epithelial Volt/Ohm Meter, World Precision Instruments, Inc.). Confluent cell layers with TEER values greater than 300 Ω·cm² were used for permeability and cytotoxicity studies. A dispersion of 1 mg/mL DIR-loaded NPs or a solution containing an equivalent concentration of soluble DIR was prepared in phenol-free HBSS (Life Technologies) containing 25 mM glucose and added to the apical chamber of the transwell filter. HBSS in the basolateral chamber was sampled and replaced with fresh media at each time point. The rate of cumulative DIR transport to the basolateral chamber gave the flux, dQ/dt. The apparent permeability (Pₐₚₚ) was calculated by (1).

$${P}_{app} = \frac{dQ / dt}{{C}_{0} \times A} \qquad Eq , 1$$

where C₀ is the initial concentration of total DIR in the apical chamber and A is the area of the transwell filter.

TGR5 binding studies. The competitive binding of pUDCA, UDCA, and PLGA NPs to macrophages saturated with an Atto565-conjugated TGR5 antibody was performed. The cells (10¹ cells/well) in 96 well plates were incubated with an access amount (4 μg/mL) of the fluorescently labeled TGR5 antibody at 4° C. for 2 h and subsequently exposed to different concentration of NPs. At 2 h post incubation, the cells were washed three times with PBS and the number of Atto565-TGR5 antibodies bound on the cells were measured using a plate reader. The specific and non-specific k_{d} were calculated by non-linear fitting using a site saturation total binding equation, Y=Bₘₐₓ×X/(k_{d}+X)+NS×X, where Bₘₐₓ is the maximum specific binding, k_{d} is the equilibrium dissociation constant, and NS is the slope of nonspecific binding. To study valency dependent NP binding to TGR5 on pancreatic β cells (10⁶ cells/well), UDCA monomer was biotinylated and conjugated onto avidinated PLGA NP surface. PLGA (100 mg) in 2 mL chloroform was added dropwise to a mixture of avidin-palmitate in PBS (10 mg/2 mL) and 5% PVA 2 mL, and homogenized. UDCA was conjugated with biotin (1:1 molar ratio) using the EDC/NHS chemistry prior to immobilization of biotinylated UDCA (0, 50, 250, and 1000 ng/mL) to avidinated PLGA NPs (5 mg/mL). To prepare plated TGR5, recombinant TGR5 receptor (5 g/mL) was coated on the plate overnight and the non-specific binding sites were blocked using a protein blocking buffer. The TGR 5 receptors on the plates were incubated with an access amount (4 μg/mL) of the fluorescently labeled TGR5 antibody at 4° C. for 2 h and subsequently exposed to different concentration of NPs. At 2 h post incubation, the plates were washed three times with PBS and the number of Atto565-TGR5 antibodies bound on the receptors were measured using a plate reader.

Data was fit to the following competitive inhibition equation, (using Graphpad Prism), which gave an estimate of pUDCA at which 50% of the labeled antibody is competed off (EC₅₀) and its affinity constant (Kᵢ):

$$F = {F}_{initial} + [ \frac{( {F}_{Final} - {F}_{initial} )}{( 1 + {10}^{[ pUDCA ] - Log ( {EC}_{50} )} )} ] \qquad Eq . 2$$

$$Log ( {EC}_{50} ) = Log [ {10}^{{logK}_{i} * ( 1 + \frac{{C}_{Anti}}{{K}_{D , Anti}} )} ] \qquad Eq . 3$$

where
F=Fluorescence change with competition against labeled TGR5 antibody;
   [pUDCA]=Concentration of pUDCA;
   F_{Initial}=Upper plateau of fluorescence or initial fluorescence;
   F_{Final}=Lower plateau of fluorescence or final fluorescence;
   EC₅₀=Concentration of pUDCA that lowers the total fluorescence by 50%;
   Kᵢ=pUDCA affinity constant;
   C_{Anti}=Concentration of labeled anti-TGR5 antibody;
   K_{D,Anti}=Affinity constant of the labeled anti-TGR5 antibody to TGR5 receptors estimated in the nanomolar range.

It was estimated that a shell of thickness equivalent to the molecular diameter of an UDCA can incorporate approximately 2000 UDCA monomers on a 350 nm diameter pUDCA particle.

Quantitation of cellular endocytosis and exocytosis rates of NPs. BMMs were seeded in a 96 well plate (10⁵ cells/well) and DIR-loaded pUDCA, PLGA, and PLGA/pUDCA blend NPs (100 μg/mL) were added to the media. Cells were incubated for 1, 3, and 6 h at 37° C., and endocytosis of NPs was measured using a plate reader. After washing the cells and replacing with new media, exocytosis of NPs was monitored at 37° C. or 4° C. by measuring released DIR-labeled NPs from BMMs to media over time.

The equilibrium endocytosis-exocytosis reaction can be simplified to:

$$[ P ] + [ C ] {⇌}_{{k}_{exo}}^{{k}_{endo}} [ PC ] \qquad Eq . 4$$

where [P]=concentration of particles in the media (number of particles/mL)
   [C]=concentration of cells in the media (number of cells/mL)
   [PC]=concentration of particles associated with cells (number of particle-cell/mL)
   kₑₓₒ=rate of exocytosis (t⁻¹)
   k_{endo}=rate of endocytosis (([P]·t)⁻¹)
then,

$$\frac{d [ PC ]}{dt} = {k}_{endo} [ P ] [ C ] - {k}_{exo} [ PC ] \qquad Eq . 5$$

In terms of a signal reporting on the endocytosis and exocytosis process, which is a fluorescence signal associated with each process [S].

$$\frac{dS}{dt} = {k}_{endo} [ P ] S - ( {k}_{endo} [ P ] + {k}_{e x o} ) S \qquad Eq . 6$$

This differential equation has a solution between the two extreme limits of no uptake to maximal uptake:

$$S = \frac{{k}_{endo} [ P ] {S}_{max} [ 1 - {e}^{- ( {k}_{endo} [ P ] + {k}_{exo} ) S}}{{k}_{endo} [ P ] + {k}_{exo}} + {S}_{0} \qquad Eq . 7$$

S₀=signal at an arbitrary start time t₀

This analysis and fitting for k_{endo} and kₑₓₒ are done using two plots.

### Exocytosis Phase

$$\frac{dS}{dt} = - {k}_{exo} S so Ln ( \frac{{S}_{0}}{{S}_{t}} ) = {k}_{exo} t \qquad Eq . 8$$

where St is the signal at any time (t)
S0 is the signal at an arbitrary time (t₀)

### Association Phase

The association phase is analyzed in terms of two plots:

$$dS / dt against S gives \qquad Eq . 9$$

$$Slope = - ( {k}_{endo} [ P ] + {k}_{e x o} ) , Intercept = {k}_{endo} [ P ] {S}_{max} at \frac{dS}{dt} = 0$$

and as mentioned above Ln (dS/dt) against t gives

$$Slope = - ( {k}_{endo} [ P ] + {k}_{exo} ) , \qquad Eq . 10$$

$$Intercept = Ln ( {k}_{endo} [ P ] {S}_{max )} ) at Ln ( \frac{dS}{dt} ) = 0.$$

Assumption: This analysis does not take account of particle re-uptake after exocytosis.

Flow cytometry and ELISA. CD44+CD8+ cells and CD4+CD25+Foxp3+ Tregs were acquired at 3- and 5-day post CY treatments for CY-induced mice. For spontaneous T1D animal model, T cells were collected at day 1 after the last NP dose. In both cases, pancreatic lymph nodes were harvested and processed using a 40 μm cell strainer. Cell surface markers were ascertained with fluorescent antibodies for CD8 (APC), CD44 (PE), CD4 (APC), and CD25 (Alexa Fluor-700) by incubating for 30 min at 4° C. Cells were then fixed, permeabilized, and stained for Foxp3 (PE) using the Foxp3 staining kit (eBiosciences). After the final wash, samples were immediately run on an LSR-II multicolor flow cytometer (BD Biosciences) and analyzed using FlowJo software (Tree Star). To study antigen-specific T cell responses, OVA-specific CD4+ cells were used in OTII co-culture assays. BMDCs (2.5×10⁴ cells/well, 96 well plate) were pretreated with pUDCA NPs for 24 h, washed, and then stimulated with LPS (10 ng/mL) and OVA (20 μg/mL) for 24 h, followed by co-culture with OTII CD4+ T cells (5×10⁴ cells/well, 96 well plate) for 3 days. Cell proliferation and cytokine production were then quantified.

BMMs (10′ cells/well, 96 well plate) were incubated with pUDCA NPs (50 μg/mL), UDCA monomer (50 μg/mL), PLGA (50 μg/mL), or PBS for 4 h and added with CpG (100 ng/mL). After 20 h, media supernatant was collected for IL-6, IL-10, and CCL1 readouts. Cells were stained for F4/80 (Alexa Fluor-647), CD86 (PE), and CD206 (FITC). After 3 washes with buffer (2% FBS in PBS), samples were fixed in 2% paraformaldehyde and run on an Attune NxT multicolor flow cytometer (Life Technologies). CD11c-F4/80+NP+ was used for NP tracking of macrophages with dye-loaded particles. The NP+ designation refers readout of the particle via fluorescence of loaded dye. Mice were fasted for 4 h and treated with labeled pUDCA NPs by oral gavage (500 mg/kg, 250 μL). After one day, pancreas, liver, lungs, and spleen were harvested and gently processed with a homogenizer and using a 40 μm cell strainer and plunger for separations of cells from debris. Cell surface markers were stained with fluorescent antibodies for F4/80 (Alexa Fluor-700) and CD11c (PE-Cy7) and measured by Attune NxT multicolor flow cytometer. All antibodies were diluted 1:500 for flow cytometry and cytokines (IL-6, IL-10, and IFNγ) were measured by ELISA (BD Biosciences).

Biodistribution and histology. B6, NOD, or Nude mice were fasted for 4 h and treated with DIR- or coumarin 6-encapsulating NPs by oral gavage (50, 100, or 500 mg/kg, 250 μL). Free DIR or coumarin 6 (solubilized with 1% Tween 20) served as controls. Mice were sacrificed at time points of 4, 8, 12, or 24 h post gavage, and a Bruker molecular imaging instrument (Carestream Health, Inc.) was used to scan organs ex vivo to measure fluorescence intensity. Fluorescence data was fit to the one component exponential decay model: Y=Y_{f}+(Yₒ−e^{-kt}). DIR-loaded NPs formulated by pUDCA, PLGA or the mixture (50:50, w/w) were also intravenously (IV) administered (100 mg/kg, 50 μL) to mice via tail vein injection to compare their biodistribution with free dye. Macrophage Depletion Kit (Clodrosome®, Encapsula NanoSciences, 100 mg/kg, intraperitoneal (IP) injection) was used to deplete macrophages in B6 mice. For histology, pancreata from the mice orally received iron oxide-loaded pUDCA NPs were fixed in 10% neutral buffered formalin for histological analysis by hematoxylin and eosin (H&E) and Prussian blue stains. The stained sections were prepared by the Yale University Pathology Histology Service. Tissues were imaged on a Nikon TE-2000U microscope with a Nikon DS Fil color camera and NIS Elements AR software (version 2.30).

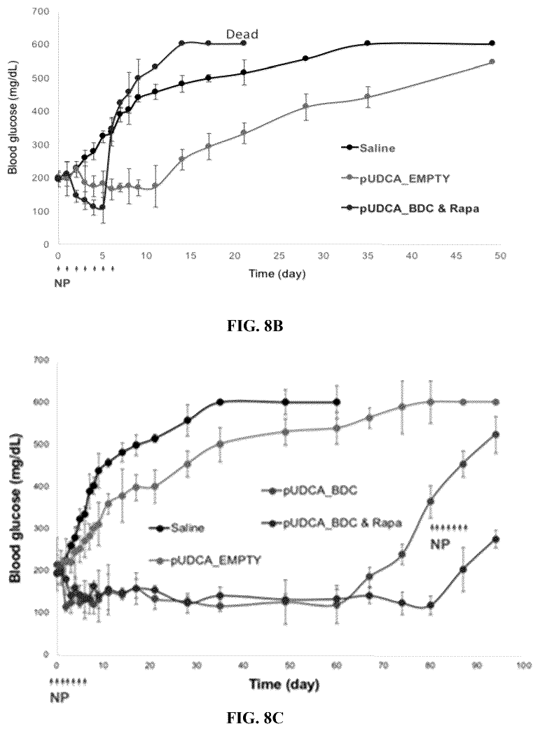

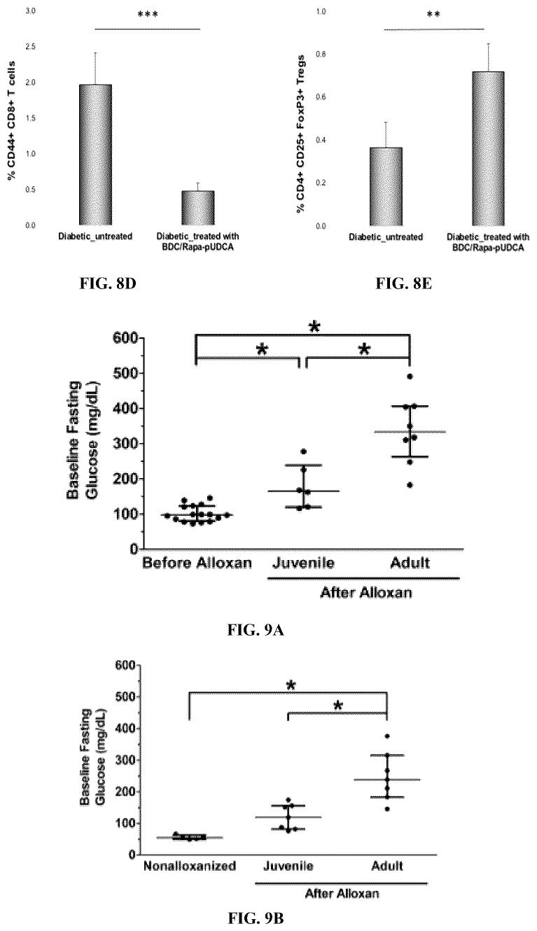

Experiments with diabetic animal models. NOD mice were intraperitoneally injected with CY (200 mg/kg) to induce acute type I diabetes (T1D) (FIG. 8A). After 24 h, the mice were orally gavaged with empty NPs, RAPA-loaded NPs (50, 100, and 500 mg NP/kg=40 mg RAPA/kg), soluble RAPA (40 mg/kg solubilized with 1% Tween 20), and saline. pUDCA_{RAPA} was orally administered at day 1 (Dose I) or twice on day 1 and 2 (Dose II). Blood glucose level was monitored using a blood glucose monitor (TRUERESULT® meter, Home Diagnostics, Inc.). Two readings (1 day apart) higher than 200 mg/dL were taken as an indication onset of T1D. pUDCA_{RAPA} was also compared to “Gold Standard” insulin administration, insulin administered subcutaneously (SQ) or intraperitoneally (IP) using this model. After 7 consecutive injections of insulin or oral gavage of pUDCA_{INS} with an equivalent insulin dose, blood glucose was measured over the course of 25 days. Data was fitted using an operational receptor depletion model, Y=Basal+(EffectₘₐₓBasal)/(1+operate), where operate=(((10^{log kA})+(10^{X}))/(10^{(log tau+X)}))ⁿ, Effectₘₐₓ is the maximum possible system response, Basal is the response in absence of agonist, kA is the agonist-receptor dissociation constant, and tau is the kinetics of lowering to half-maximal response.

For spontaneous T1D model, NOD mice were housed for approximately 2 months to allow them to naturally develop T1D. For spontaneous T1D model, NOD mice the same age (8 week old) were chosen for the study only when they became diabetic in the same week (16 week old, after 2 random tail vein blood glucose measurements of 200±15 mg/dL). The mice were orally treated with empty NPs, INS-loaded NPs (100 or 500 mg NP/kg and 285 mIU INS/kg) or free INS (285 mIU/kg) 7 times for 1 week to monitor glycosuria and body weight. Concentration of GLP-1 was measured using an ELISA kit (Invitrogen) from plasma. Swine were fasted for >12 hours with access to water and alloxan was administrated IV (250 mL, 150 mg/kg). At 10 day post alloxan treatment, the animals (n=3) were orally gavaged with pUDCA_{INS} (6.4 mg/kg equivalent to 100 mg/kg in mice) 7 times daily and monitored glucose readings using a continuous glucose monitoring system, DexCom 6 (Dexcom. Inc.). Calibration was done by ear stick with Relion Prime BG Meter (Relion) and further venous calibration was performed from whole blood samples by Antech Diagnostics.

Calculations for INS dose chosen in the spontaneous TID study. It is known that 1 international unit (IU) of INS is needed to drop blood glucose level of 50 mg/dL for human. To lower high blood glucose level (>400 mg/dL) below hyperglycemia threshold (200 mg/dL), at least 4 IUs of INS is required. That is 0.02 IUs of INS for mice according to the following equation (9).

$$HED = Animal NOAEL \times {( \frac{Weight of animal ( kg )}{Weight of human ( kg )} )}^{( 1 - 0. 67 )} \qquad Eq . 11$$

where HED=human equivalent dose (mg/kg), Animal NOAEL=No Observed Adverse Effect Levels for animal (mg/kg), 1 IU of INS=0.036 mg based on the World Health Organization conversion factor.

The loading of INS in pUDCA NPs was approximately 20 ng/mg of NP and it is 5.6×10⁴ IU/mg of NP. In order to feed 0.02 IUs of INS, 35 mg of pUDCA_{INS} NPs should be treated to single mouse. Therefore, diabetic mice were treated with 3 doses of 10 mg of NPs and continued to treat the mice with additional 4 doses to keep the glucose level low (total 0.04 IUs of INS and 70 mg of NPs).

Toxicology of pUDCA and UDCA. Acute toxicity studies were performed with 10-week old, female B6 mice. Mice were orally dosed with pUDCA (100 and 500 mg/kg) and UDCA (100 mg/kg) at day 0. Kits from Teco diagnostic were used at day 3, 5, and 7 for analysis of serum concentrations of alkaline phosphatase, alanine transferase, total bilirubin, and blood urea nitrogen concentrations. EDTA anticoagulated blood was analyzed by a Hemavet blood counter (Drew Scientific).

Statistics. All statistical analyses were performed using GraphPad Prism software (version 7.01). Experimental comparisons with multiple groups used ANOVA analysis with Bonferroni's post test or Two-tailed Student's t tests were performed. Log-rank test and χ² statistical analysis was performed for survival data. A P value of 0.05 or less was considered statistically significant.

### Results

Fabrication of bile polymer solid biodegradable NPs. To create BA carriers, biodegradable pBAs were first synthesized then the polymers were formulated into NPs. This methodology, as opposed to the use of individual BA monomers, was for four reasons: 1) To increase stability of the BA drug combination (if present) during digestive transit in comparison to micellar vesicles that form above the BA micelle concentration. 2) To formulate carriers that can deliver an encapsulated drug (if present) in a sustained fashion. 3) To ensure a constant ratio of BA to encapsulated drug (if present) during the delivery and release process. 4) To increase the valency of the BA for potential increased avidity binding to BA receptors.

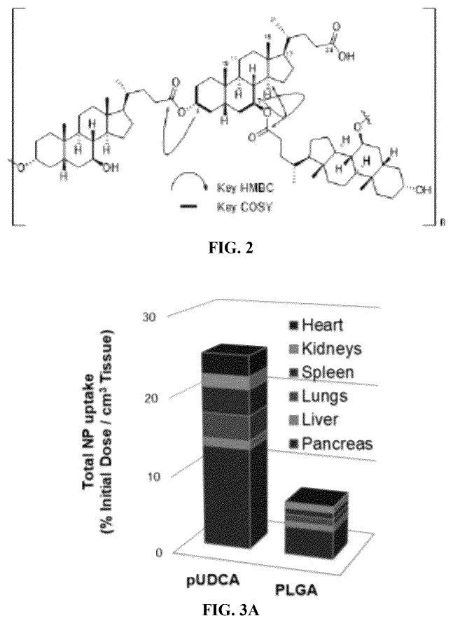

Polymerization of BAs and formulation into NPs. For screening purposes, a panel of BAs which included cholic acid (CA), chenodeoxycholic acid (CDCA), deoxycholic acid (DCA), lithocholic acid (LCA), and ursodeoxycholic acid (UDCA) was tested (FIGS. 1A-1E). BAs were polymerized under mild conditions, 40° C. and atmospheric pressure for 2 h (see METHODS). Use of diisopropyl carbodiimide (DIC) and a 1:1 salt of dimethylaminopyridine and p-toluenesulfonic acid (DMAP/PTSA) led to selective activation and esterification at the carbon-24 position of the BAs yielding BA polymers (FIGS. 1F-1J). Polymerization proceeded from the negatively charged monomer termini. The extent of polymerization and molecular weight were ascertained by nuclear magnetic resonance (NMR) and gel permeation chromatography (GPC), respectively. Polymerization and crosslinking were validated by two-dimensional (2D) heteronuclear single-quantum coherence (HSQC) spectroscopy and 2D double-quantum-filtered correlation spectroscopy (DQF-COSY) analyses (FIG. 2). In general, the number average molecular weight (Mn) range was 1360-2230 g/mol, and the weight average molecular weight (Mw) was in the range 1600-3210 g/mol. Polymer dispersity was assessed with a polydispersity index (PDI) and did not exceed 1.5 (Table 1). Linearity of the polymer was assessed by 2D NMR where it was shown that two hydroxyl substituents at C-3 and C-7 were esterified with 2.5:1 molar ratio during the polymerization process.

A water-in-oil-in-water (W/O/W) double-emulsion methodology was used for nanoparticle formulations (FIGS. 1H-1J). The polyester, PLGA, was used here as a comparator for drug delivery. Blends of pBA and PLGA were fabricated to demonstrate the relative impact of pBA on PLGA function. Encapsulation of agents was achieved by first initial dissolution of the polymer in chloroform then during emulsion formulation agents were added. The agents utilized in this study include: infrared fluorescent dye (1,1′-dioctadecyl-3,3,3′,3′-tetramethylindotricarbocyanine iodide (DIR), green fluorescent dye (coumarin 6), the drug rapamycin (RAPA), 10-20 nm iron oxide particles used for histological staining with the iron sensitive Prussian blue, and saline buffered mouse insulin (see METHODS). Loading efficiencies of DIR, coumarin 6, RAPA, iron oxide, and insulin were 6.7, 84.3, 80.1, 9.0, and 0.02 g per mg of particles, respectively. Spherical morphology of the NP was validated by scanning electron microscopy (SEM) which showed uniformly spherical particles with an average diameter of 344.3±4.7 nm quantitated using dynamic light scattering (DLS, Zetasizer, Malvern Instruments) (Table 1). The pBA NPs electrostatic charge was negative with a zeta-potential of −24.9±4.4 mV. All preparations were engineered to have the similar size, charge, morphology and encapsulation efficiency to ensure that any observed biodistribution or functional differences were strictly a function of intrinsic material properties and not biophysical variations (Table 1).

TABLE 1
Particle properties and loading efficiency.

| | | | | | | | Dye | Loading |
|---|---|---|---|---|---|---|---|---|
| | | | | Mean | | Zeta-pot-al | loading | efff-y |
| | Mn^{a} | Mw^{b} | PDI^{c} | diameter (nm) | PDI^{d} | (mV) | (%)^{e} | (%)^{f} |
| PLGA | 2451 | 4184 | 1.707 | 328.8 ± 3.4 | 0.304 | −27.5 ± 2.8 | 0.699 ± 0.045 | 69.9 ± 4.5 |
| pCA | 1972 | 2962 | 1.502 | 360.3 ± 11.2 | 0.296 | −24.6 ± 3.1 | 0.702 ± 0.012 | 70.2 ± 1.2 |
| pLCA | 1357 | 1598 | 1.177 | 337.9 ± 21.0 | 0.276 | −27.1 ± 10.4 | 0.730 ± 0.020 | 73.0 ± 2.0 |
| pDCA | 1842 | 2523 | 1.370 | 311.9 ± 24.1 | 0.213 | −22.7 ± 1.5 | 0.687 ± 0.064 | 68.7 ± 6.4 |
| pCDCA | 1741 | 2284 | 1.312 | 335.1 ± 9.8 | 0.011 | −27.8 ± 10.1 | 0.687 ± 0.014 | 68.7 ± 1.4 |
| pUDCA | 2225 | 3210 | 1.443 | 344.3 ± 4.7 | 0.164 | −24.9 ± 4.4 | 0.674 ± 0.005 | 67.4 ± 0.5 |
| Blend | — | — | — | 299.5 ± 14.3 | 0.131 | −22.2 ± 5.6 | 0.726 ± 0.019 | 72.6 ± 1.9 |
| (pUDCA/PLGA)^{g} | | | | | | | | |

^{a}The number average molar mass (GPC)
^{b}The weight average molar mass (GPC)
^{c}Polymer polydispersity index (GPC)
^{d}Particle polydispersity index (DLS)
^{e}(weight of encapsulated dye/weight of NPs) × 100
^{f}(weight of encapsulated dye/weight of dye used for encapsulation) × 100
^{g}Polymer blend nanoparticles (pUDCA:PLGA = 50:50, w/w).

Improved GI Transport: Stomach Protection and Enhanced Intestinal Permeation.

BA ionization under acidic conditions impact its water solubility, limiting water penetration into individual particles while promoting hydrophobic interactions between particles in acidic conditions. This protective mechanism, which limits exposure of the majority of particles to the stomach milieu, reverses with increased pH conditions in the intestinal lumen.

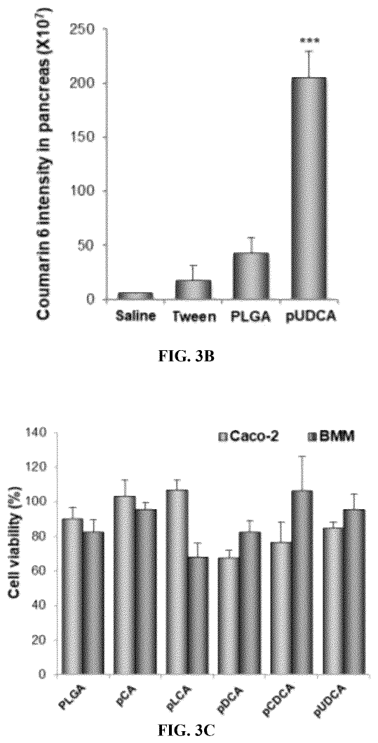

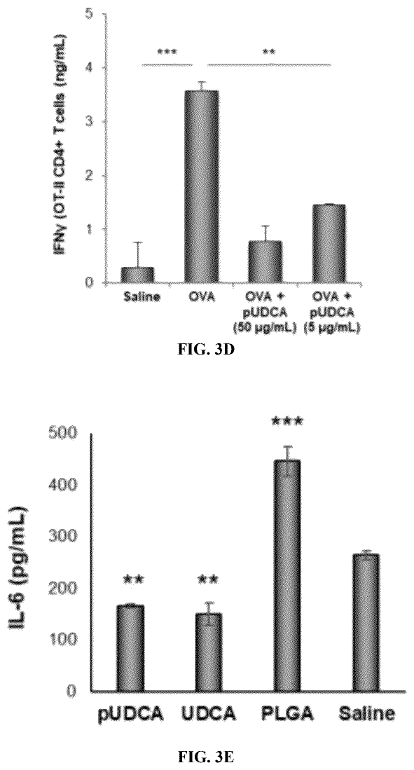

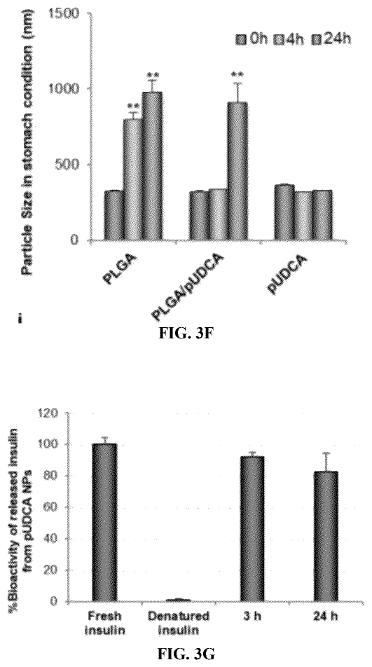

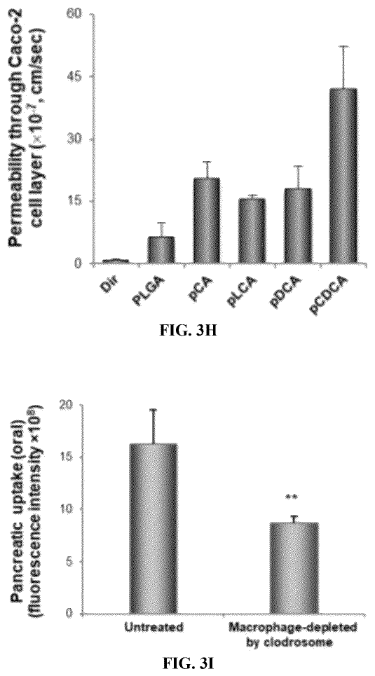

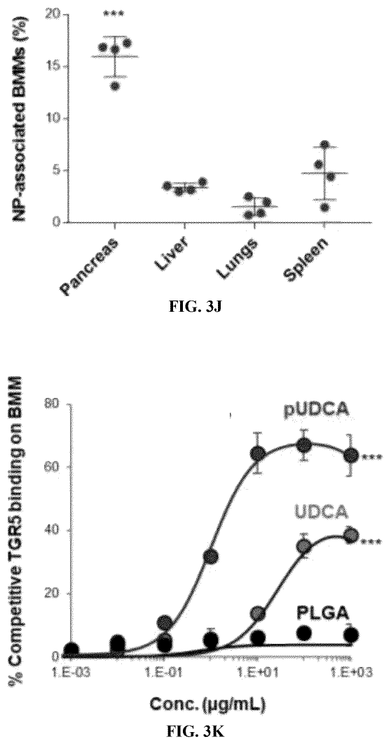

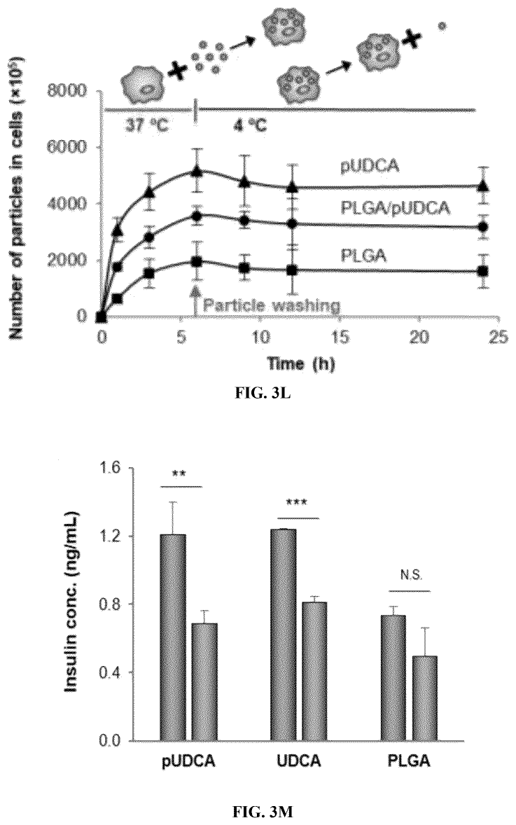

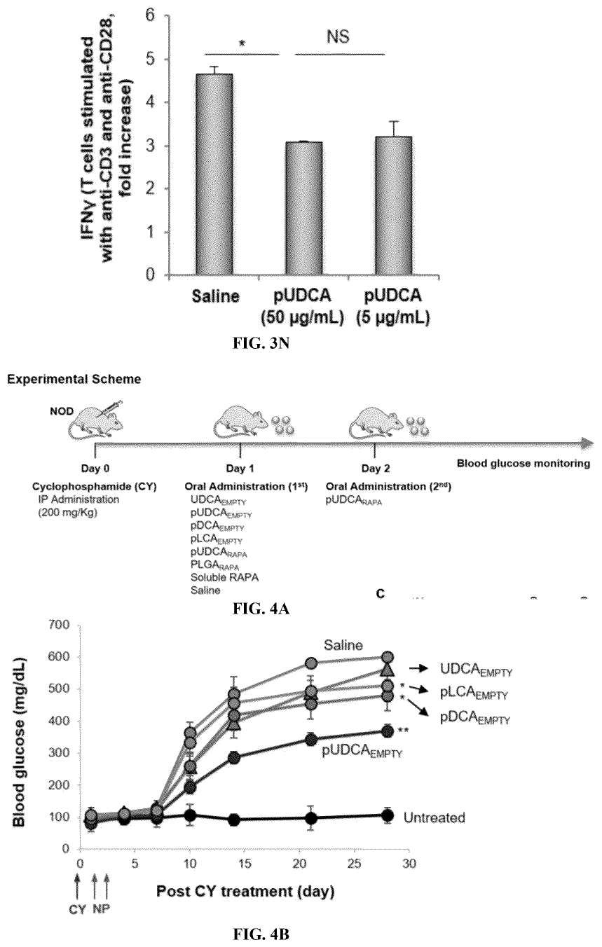

PLGA particles began to degrade at 2 and 4 h post incubation, releasing up to 8% of the encapsulated dye. In contrast, pUDCA retained dye over 4 h in a manner similar to Eudragit modified PLGA. PLGA blended with pUDCA imparted a Eudragit comparable stability which further validated the pH responsive properties of pUDCA. The spherical particulate morphology of pUDCA was also retained while that of PLGA showed significant swelling over time in acidic conditions (FIG. 3F). Blending PLGA with pUDCA had a transient stabilizing effect. These observations speak to the protective nature of pBAs, which impart stability on encapsulated insulin during GI trafficking. Compared to UDCA micelles, pUDCA showed minimal leakage of encapsulated insulin. Furthermore, insulin encapsulated in pUDCA was just as functional as fresh insulin after low pH exposure as demonstrated by that insulin released from pUDCA_{INs} showed an ability to bind insulin receptors on CHO cells producing pAkt (FIG. 3G).

Following stomach digestion, NP encounter a higher pH microenvironment and become more water permeable. BA particle permeation through the intestinal lumen can take place via passive transcytosis through intestinal epithelial cells or active transport via engagement of colonic receptors. pUDCA appears to transport passively through the intestinal lumen since the permeability of dye-loaded pUDCA NPs through a model epithelial cell line (Caco-2 human cell) was significantly more than PLGA, PLGA blended with pUDCA, or other pBA NPs (FIG. 3H). Indeed, the permeability coefficient of pUDCA is approximately 50×10−6 cm/sec which means that it will be fully absorbed in the intestinal lumen in humans63.

Blood pool entry of BAs can be mediated by either enterohepatic circulation or blood cells such as monocytes or macrophages in the intestinal lumen. To elucidate the mechanism to pUDCA NP circulation, the NP were intravenously (IV) injected and found that pUDCA NPs showed the high pancreatic uptake profile at 2 h post IV administration. Pancreatic accumulation thus also involved cell transport in the blood and was not entirely driven by enterohepatic circulation. Given that intestinal macrophages are one of the largest pools of cells in the body and are in immediate proximity to the lamina propria in the healthy colon, a reduction in the number of cells in this pool with depleting agents such as clodronate liposomes should affect pancreatic biodistribution. The effect on biodistribution is shown in FIG. 3I, supporting the partial role of macrophages in particle biotransport to the pancreas. Complete elimination of pancreatic distribution via this mechanism would negate the role of enterohepatic circulation, but FIG. 3J shows that only 16% of macrophages were associated with pUDCA NPs. The overlapping mechanisms contributing to NP circulation are summarized as follows. Here, NP permeating the intestinal epithelium enters the duodenum and can access the pancreatic duct via the common bile duct. Particles may also be uptaken by resident and circulating monocytes and macrophages to traffic to pancreas, or on their own percolate through capillaries and lymphatic vessels66 and without a cellular-host. Another potential mechanism, not investigated here, is binding to serum albumins which have affinity to different bile acids.

pUDCA NPs Bind the Extracellular Bile Acid Receptor
(TGR5) with High Avidity and Facilitate Glucagon-Like Peptide and Endogenous Insulin Secretion.

BAs engage apical membrane transporters such as CD36, caveolin, and fatty-acid transporter (FAT) to actively transport fatty foods, thus high avidity binding can enhance endocytosis and BA-mediated signal transduction cascades. Furthermore, monomeric UDCA is a weak TGR5 receptor agonist, thus understanding the changes in binding affinity would help elucidate the amplified response of polymeric BA compared to monomer. The competitive binding of pUDCA to macrophages saturated with an anti-TGR5 antibody is shown in FIG. 3K and Table 2.

TABLE 2
Dissociation constants for pUDCA, UDCA, and PLGA.

| | Specific k_{d} | Non-Specific k_{d} |
|---|---|---|
| pUDCA | 1.25 | −0.0061 |
| UDCA | 29.97 | −0.0055 |
| PLGA | N/A | ~0 |

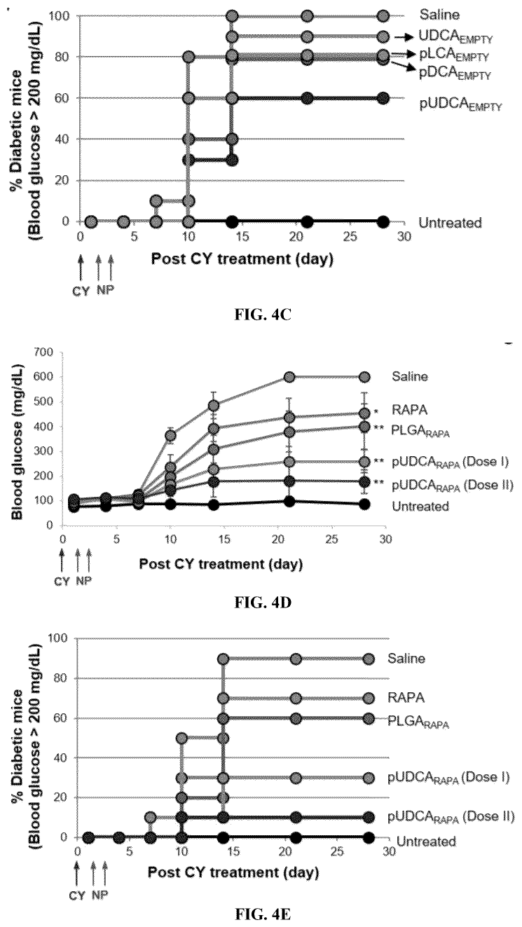

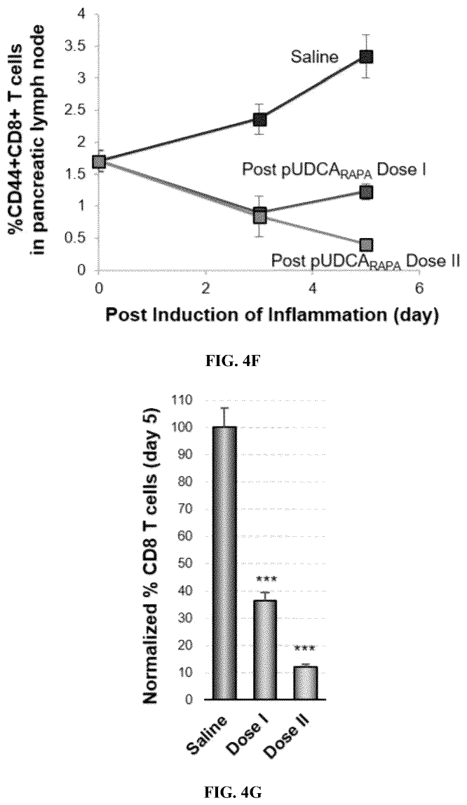

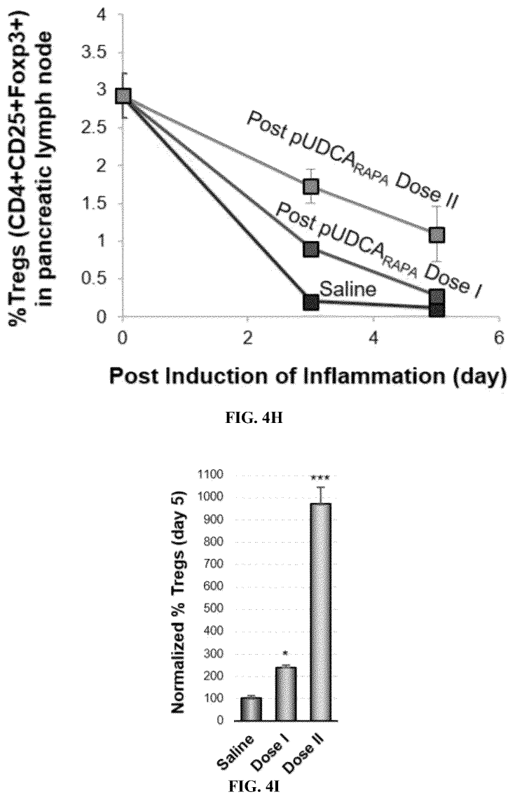

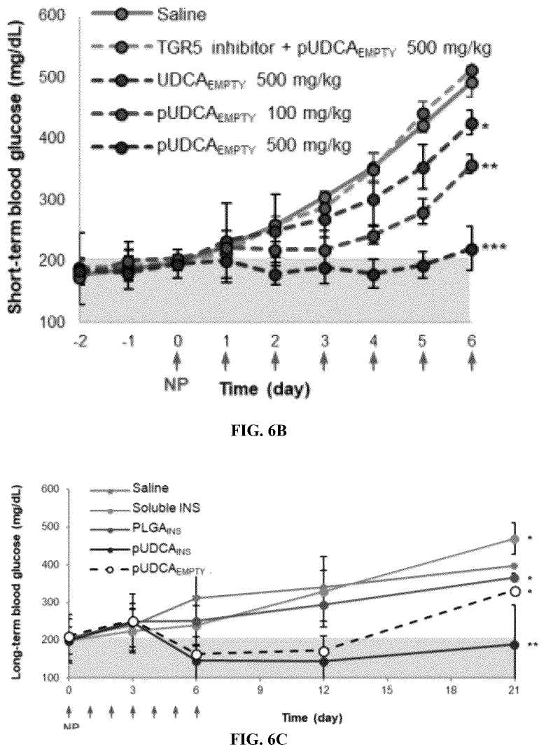

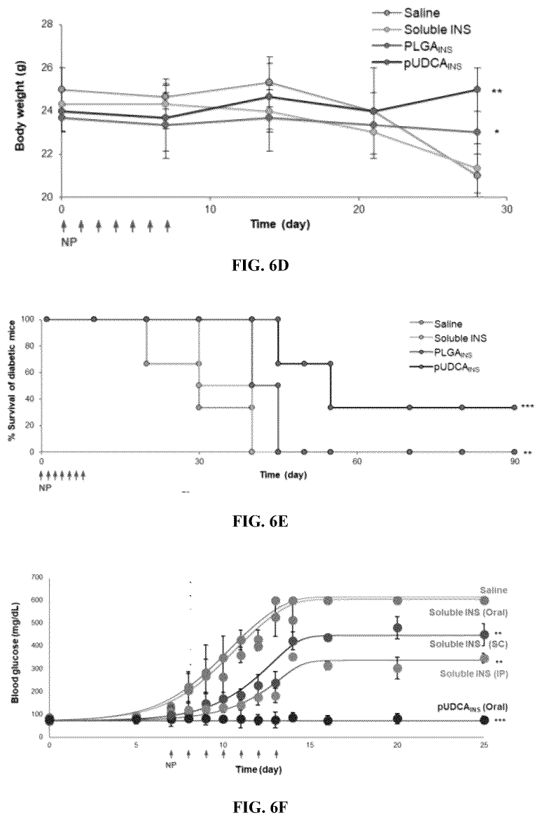

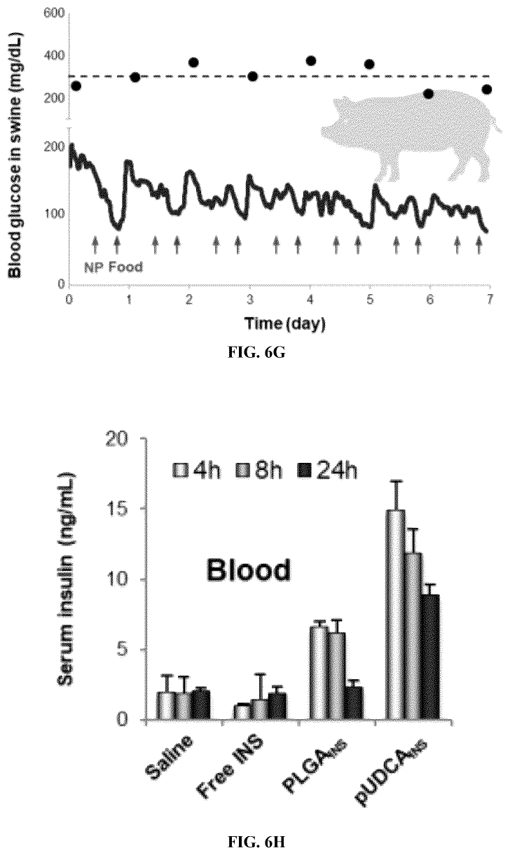

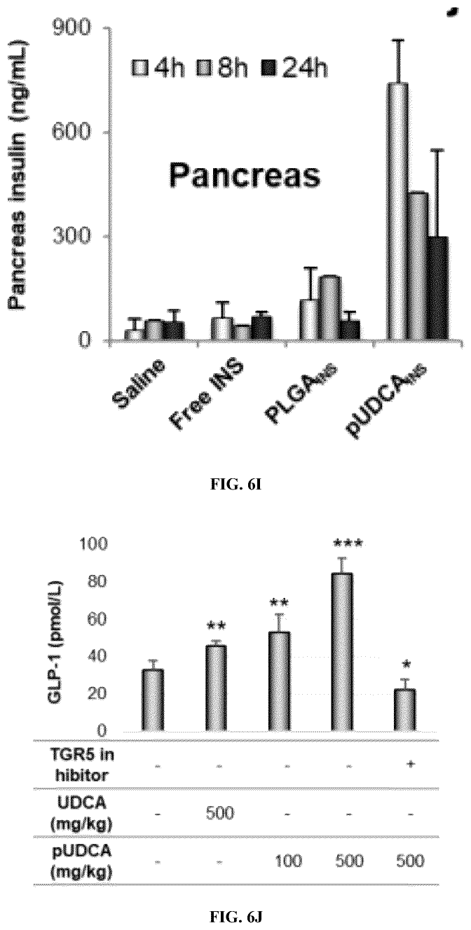

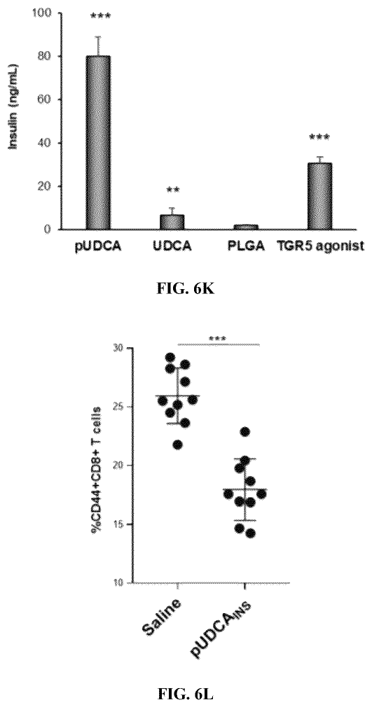

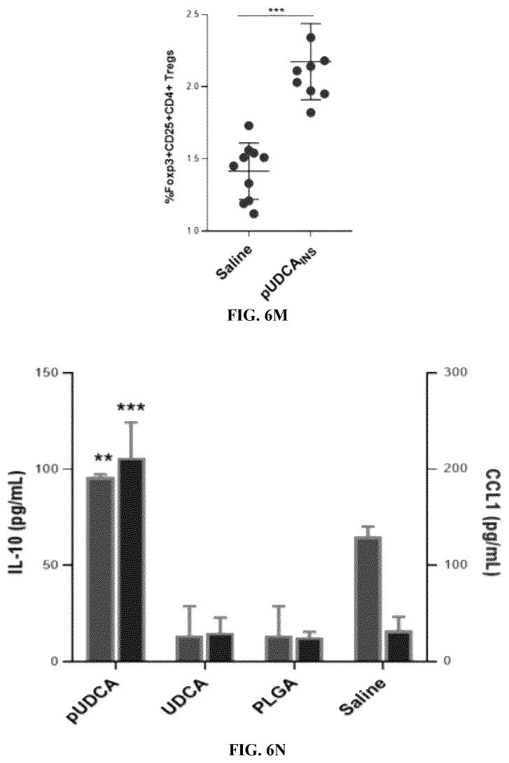

Compared to UDCA, the affinity of pUDCA was about 30 fold greater with minimal non-specific binding. PLGA NP used as a negative control showed no affinity. Rates of intracellular NP trafficking are also impacted by higher binding avidity. A quantitative assessment of pUDCA NP rate of endocytosis and exocytosis in macrophages at 37° C. showed internalization, (kendo), was 3.6 fold higher compared to non-binding control (PLGA) and the rate of exocytosis, (kexo), was 2.3 fold faster. The assay validity in this quantitative determination of rates was performed at 4° C. where minimal or no active internalization or exocytosis is shown (FIG. 3L). Interestingly, elevation in the magnitude of internalization or faster exocytosis depended on the amount of pUDCA in the formulation. A 50% reduction in the pUDCA concentration, by blending with PLGA, led to an internalization rate or exocytosis rate that was greater or faster respectively compared to PLGA itself. Thus, increased binding avidity of pUDCA led to increased rates of receptor-mediated intracellular transport (endocytosis and exocytosis). Indeed, high avidity binding was not exclusive to only macrophages but was also observed in pancreatic β cells where TGR5 binding results into enhancements in endogenous GLP-1 secretion (FIG. 6J) and insulin production (FIG. 6K) facilitating effective control over blood glucose levels. In addition, to metabolic control, increased binding avidity also makes possible an intrinsically potent anti-inflammatory immune response control by engagement of multiple immunomodulatory machineries, for example, through macrophage phenotypic skewing from M1 to M2 (FIG. 6O), reduction of IFNγ secretion (FIG. 3D), reduction of pro-inflammatory IL-6 (FIG. 3E), augmentation of anti-inflammatory activity with production of IL-10 and CCL1 (FIG. 6N), and induction of tolerance via regulatory T cell response (CD4+Foxp3+CD25) proliferation in parallel with suppression of the activated CD8+(CD44+CD8+) T cell response (FIGS. 4F-4I, 6L and 6M). This multivariate innate and adaptive immune modulation by the carrier material itself offers significant therapeutic control over pancreatic inflammation in conjunction with provisioning synergistic drugs towards neutralization of the pro-inflammatory response associated with hyperglycemia.

pUDCA Effects are Mediated Through Enhancement in Valency and Proximity of Multivalent Display

The anti-inflammatory effects of UDCA have been widely studied and exploited medicinally for dissolution of gallstones and prevention of chronic graft versus host disease in the liver. In Chinese, the word “Urso” means “bear” and ursodeoxycholic acid is a multibillion-dollar industry in Chinese traditional medicine, owing to its anti-inflammatory properties and general benefits in healthy and sick populations. Additionally, clinical trials are ongoing focusing on UDCA's role in treatment of diabetes. pUDCA is a multivalent form of UDCA that owes its enhanced effects to high avidity binding to the TGR5 receptor in addition to functioning as a carrier.

To demonstrate that increased multivalency is the mechanism behind the observed effects, the pUDCA effect on insulin secretion in vitro from pancreatic β cells was tested. PLGA, a carrier with no intrinsic effect on insulin secretion from pancreatic cells (FIG. 6K), was modified with different concentrations of monomeric UDCA up to full saturation of the surface which was approximately 2000 molecules of UDCA per PLGA particle. While an increase in UDCA concentration on the surface potentiated the binding of a non-pUDCA carrier (i.e. PLGA) to target pancreatic cells, the effect of pUDCA was still superior to PLGA particles saturated with monomeric UDCA (up to 2000 UDCA molecules per PLGA particle). Thus, the enhancement effect cannot be fully recapitulated by increasing only the monomeric valency on a nanoparticle. Additional geometrical or biophysical factors are required, and these can range from the proximity of the molecules on the multivalent platform, to mobility or relative persistence of ligands on the activating surface. Consistent with the notion that increased affinity alone is often not a sufficient parameter to achieve optimal responses, the spacing between proteins needs to be accounted for the design of multivalent targeting systems. Polymerized ligands can inherently span the lengths for targeting BA receptors because the composition is a distribution of randomly spaced ligands that span a wider set of length scales in comparison with monomers conjugated or adsorbed to the surface of a solid support or nanoparticle. In addition, pUDCA encapsulating agent allows for the provision of a fixed ratio between bile acid and released drug over a time span needed to increase both moieties for improved response. The surface of a particle decorated with monomer will be degrade with kinetics that are different from the release kinetics of the encapsulated agent. If combination delivery of a pair of pluripotent drugs is required for therapy, then the temporal stability of the combination plays a critical role. Temporal stability is a pre-requisite to potential synergistic activity and that would be best achieved via a system that provisions the active drugs at a fixed ratio over time. This can be easily accomplished if the carrier is one of the two effector drugs (the bile acid and encapsulated agent).

The multivariate properties of this carrier are summarized as follows. After oral ingestion (pH-7-7.5), particles are anionic and dispersible. Protonation in the stomach (pH 2-3) limits water penetration. The small intestine pH (6-6.5) reduces hydrophobic interactions and enable water penetration in particles, and transport through intestinal lumen, followed by binding and internalization by intestinal macrophages. In summary, particles circulate systemically by a number of mechanisms, involving either cell-mediated transport or particle trafficking alone from the duodenum to the common bile duct facilitated entry into the exocrine pancreas. The particles show protective and localized transport through the GI tract, metabolic and immunological control over the response. Together these properties are a means to potentiate the effects of an encapsulated anti-inflammatory or metabolic therapeutic.

Excipient-mediated agent delivery has been met with a plethora of different innovative designs for oral delivery and enhanced function. Examples include: protective excipients, macromolecular transporters, nanoparticles, polymeric scaffolds, and millimeter microneedle-like pills that directly deliver insulin through the gastric wall. Yet with these innovations, no approaches exist that address the metabolic management aspect of agent delivery in parallel with strategies that treat the underlying pathology, e.g., the dysfunction in pancreatic metabolism and local pro-inflammatory immunity in type 1 diabetes. The absence of such development thus necessitates a life-time reliance on insulin administration.

The Examples show that polymeric BAs not only facilitate the formulation of orally ingestible therapeutic nanoparticles but also provide a broad-spectrum of bioactivity for two reasons:
1) they can be protective in nature, and increase intestinal permeation and thus the systemic bioavailability of associated agents; and
2) they possess signaling functions that can regulate glucose metabolism and immunity through binding of BA receptors and thus function as effector therapeutic systems.

The rationale for polymerization was based on the notions that: 1) polymerization facilitates a strategy for encapsulation and release of a wide range of therapeutics of interest including insulin. In other words, solid, stable, biodegradable polymeric carriers in contrast to monomeric BA micelles, which are inherently unstable. 2) Fabrication of such polymeric NPs enable sustained release of encapsulated agents if the polymers are degradable in aqueous environments. 3) Polymeric BA systems as robust carriers present BA differently (in close proximity and higher density) than, say, BA monomers hybridized on the surface of another type of polymer NPs. Furthermore, if the BA monomer has intrinsic therapeutic effect, then this effector function is be amplified with polymerization and its bioavailability is longer lasting in contrast to BA monomers on particles which may be easily released from the surface after oral ingestion. Furthermore, the sustained availability of BA over the drug release period may be a desired element for combinatorial synergistic activity. 4) The pH stimulus response of BAs which is due to their ionization potential and protonation at low pH offers stomach protection while enhancing BA with multivalency for binding to its receptors. This multivalent response not only amplifies the degree of ionization, but also kinetically amplifies the low pH protection and higher pH deprotection response time as particles transit from the stomach to the intestinal milieu. 5) Polymeric multivalency results in high binding avidity to BA receptors which results in conversion of a weak BA agonist into a stronger form upon polymerization. Stronger agonists enable greater receptor activation and therapeutic signaling functions at lower doses.

For the above reasons, presented are the uses of the polymers formed from the BA monomer ursodeoxycholic acid (UDCA). UDCA has an established record of use for lowering insulin resistance in Type 2 Diabetes (T2D), however this usage is dose-dense (typically 40-450 mg/kg in mice and for 2-20 weeks orally). UDCA is rarely tested in T1D since it mainly impacts insulin sensitivity. The functional impact of pUDCA extends beyond improvements in transport of encapsulated agent (such as insulin) in addition to amplification of its effector function beyond what the monomer can achieve on its own. UDCA can trigger protein kinase cascade cell activation, regulate glucose, and energy homeostasis if it stably binds to extracellular Takeda G-protein coupled receptors (TGR5), and can regulate nuclear factor κB (NF-κB) and signal kinases such as protein kinase B (Akt). TGR5 activation also results in anti-inflammatory immunity, anti-fibrotic activity, induction and secretion of GLP-1 from enteroendocrine L cells together with increased energy expenditure in adipose tissue. pUDCA may not only significantly lower the dose but amplify the range of UDCA function because its monomeric counterpart, UDCA, is an intrinsically weak TGR5 agonist.

From the standpoint of improvements in insulin transport, biodistribution and pharmacokinetics, BAs are natural emulsifiers. Thus, biodegradable, polymeric BA would be even better in solubilization of lipids and fats in the body. Generally, BAs function as digestive aid through their self-assembly with lipids into micelles; enabling better molecular biodistribution and blood circulation of orally ingested fatty substances. Bile and pancreatic digestive juices are known to secrete into the duodenum and bile, specifically, is shuttled from the ileum back to the liver through portal circulation then once again returned back to the intestines for further digestion of ingested fatty foods. This circulatory action of BAs from the intestines to the bile duct and back is a process termed, “Enterohepatic Circulation”.

Because of the enhanced binding of polymeric BA NP, biodistribution is affected and circulation lifetime is longer. While previous work linking drugs covalently or non-covalently to BAs have exploited this cycling process towards enhanced intestinal permeation and drug pharmacokinetics, no reports have explored or documented the multimodal therapeutic aspects of polymerized BAs, especially pUDCA, in conjunction with loaded insulin for the management, prevention and treatment of T1D.

### Methods

The methods used in the Examples were as follows.

Reagents and antibodies. All bile acids, para-toluenesulfonic acid, 4dimethylaminopyridine (DMAP), poly(vinyl alcohol) (PVA), Tween 20, pepsin, triamterene, lipopolysaccharide (LPS), and ovalbumin (OVA) were obtained from Sigma and Sigma-Aldrich. Cyclophosphamide (CY), anhydrous methylene chloride, anhydrous pyridine, diisopropyl carbodiimide, and anhydrous methanol were purchased from ACROS. Poly(lactic-co-glycolic acid) (PLGA, inherent viscosity 0.55-0.75 dL/g, carboxyl terminal) from Durect was used as a control polymer. Rapamycin (RAPA, LC Laboratories), mouse insulin (INS, R&D systems), 1,1′-dioctadecyl-3,3,3′,3′-tetramethylindotricarbocyanine iodide (DIR, Biocompare), and coumarin 6 (ACROS) were encapsulated in NPs. Eudragit FS 30D was obtained from Evonik and CpG was purchased from InvivoGen. Antibodies for CD8 (APC), CD44 (PE), CD4 (APC), CD25 (Alexa Fluor-700), CD11c (PE-Cy7), F4/80 (Alexa Fluor-647), F4/80 (Alexa Fluor-700), and CD206 (FITC) were obtained from BioLegend. Foxp3 (PE) and CD86 were purchased from Invitrogen and eBioscience, respectively. Recombinant Human GPCR TGR5 protein, Atto565-conjugated TGR5 antibody, and blocking buffer were obtained from Abcam and used for competitive binding study.

Cells. The human colon adenocarcinoma Caco-2 cells were purchased from ATCC. The cells were cultured in Dulbecco's Modified Eagle's Medium (DMEM, Life Technologies) containing 4.5 g/L glucose, 10% Fetal Bovine Serum (FBS, Atlanta Biologicals), antibiotics (100 units/mL penicillin and 100 μg/mL streptomycin, Gibco), and 1% nonessential amino acid (NEAA, Gibco). Long bones and spleens were harvested from mice (C57BL/6 or Rag2/OTII) post cervical dislocation. Bone marrow was eluted from long bones and spleens were macerated using Roswell Park Memorial Institute (RPMI)-1640 (Life Technologies) media supplemented with 10% FBS. Red blood cells (RBCs) in the sample were lysed using ammonium-chloride-potassium (ACK) lysing buffer (Lonza). Bone-marrow derived macrophages (BMMs) were cultured in Roswell Park Memorial Institute (RPMI, Life Technologies) media with macrophage colony-stimulating factor (MCSF, 10 ng/mL, Sigma-Aldrich). Bone-marrow derived dendritic cells (BMDCs) were generated using a conventional expansion protocol in which 5×10⁵ cells/mL were plated in RPMI supplemented with 20 ng/mL GM-CSF (Sigma-Aldrich) and cultured for 5 days. On day 5, non-adherent cells were collected and cultured in GM-CSF media for an additional 2 days. CD4+ T cells were purified from splenocyte population in C57BL/6 using an EasySep™ Mouse CD4+ T Cell Isolation Kit (STEMCELL Technologies). All cells were cultured at 37° C. in a humidified atmosphere of 5% CO₂.

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO). The same experiment was performed in the presence of TGR5 antagonist, triamterene (50 μg/mL) as a control to differentiate inherent insulin production from the cells without TGR5 activation and used to normalize the results. Bioactivity of released insulin from pUDCA were measured using Chinese hamster ovary cells that were transfected with the gene to express insulin receptor (CHO INSR cells, ATCC). The released insulin from pUDCA at 3 or 24 h was incubated with CHO INSR cells for 1 h and phosphorylated protein kinase B (pAkt) level was measured by ELISA (Abcam). The pAkt production from CHO cells incubated with fresh or denatured insulin was compared to calculate % bioactivity.

Animals. C57BL/6 mice (B6, 6-8-week-old, female) were obtained from Harlan Sprague Dawley Inc. NOD mice (NOD/ShiLtJ, 8-week-old, female) and Nude mice (athymic nude, nu/nu, 7-week-old, female) were supplied by Jackson Laboratory. The mice were housed in autoclaved micro-isolator cages that were placed in a positive pressure containment rack. Ossabaw Swine (17-month-old, 42 kg) derived from the Ossabaw barrier islands were used. All experiments and maintenance were carried out according to an approved protocol from the Yale University Institutional Animal Care and Use Committee.

Polymer Synthesis and Nanoparticle (NP) formulation. Poly(bile acid)s (pBAs) were synthesized by esterification of the carbon 24 group (FIGS. 1F-1G). BAs (5.4 mmol), paratoluenesulfonic acid (0.652 mmol), and DMAP (0.652 mmol) were added in 60 mL of a 5:1 anhydrous methylene chloride to anhydrous pyridine solvent mixture and stirred at 40° C. to yield a clear solution. To the reaction mixture, 6.92 mmol of diisopropyl carbodiimide was added and the reaction was allowed to proceed for 2 h in the nitrogen atmosphere. The polyester product, pBA, was precipitated into 400 mL of cold anhydrous methanol collected by centrifugation (Centrifuge 5810R, Eppendorf) and dried to retain a white powder. Polymerization was confirmed by nuclear magnetic resonance (NMR) and gel permeation chromatography (GPC). ¹H and 2D-(COSY, DQF-COSY, HSQC and HMBC) NMR spectral data for UDCA and poly(ursodeoxycholic acid) (pUDCA) were recorded on an Agilent NMR spectrometer (Agilent) at 600 MHz with a 3 mm cold probe or 400 MHz and ¹³C NMR data was measured at 100 MHz magnetic field. Chloroform-d₁ (99.96%, Cambridge Isotope Laboratories, Inc.) was used as the deuterated NMR solvent and solvent reference signals (δ_{H} 7.25, 6c 76.98) for all of the NMR experiments. The molecular weight (MW) for pBAs (10 mg/mL in chloroform) were evaluated with GPC using a Waters HPLC system equipped with a model 1515 isocratic pump, a 717 plus autosampler, and a 2414 refractive index (RI) detector with Waters Styragel columns HT6E and HT2 in series. Chloroform was utilized as the mobile phase with a flow rate of 1 mL/min and both the columns and RI detector were maintained at 40° C. MW characteristics were determined relative to a calibration curve generated from narrow polydispersity polystyrene standards (Aldrich Chemical). Empower II GPC software was used for running the GPC instrument and subsequent chromatographic analysis. pBA or PLGA or the mixture (50/50, w/w) NPs encapsulating dyes (DIR or coumarin 6), drugs (RAPA or INS) or iron oxide were formulated using an water-in oil-in water (W/O/W) double emulsion technique (FIGS. 1H-1J). Polymers or the mixture (100 mg) was dissolved in 2 mL chloroform containing DIR (1 mg), coumarin 6 (10 mg), RAPA (10 mg) or iron oxide (1 mg). Phosphate-buffered saline (PBS, 100 μL) or the PBS containing INS (10 μg) was added dropwise to the chloroform polymer solution while vortexing and homogenized using an IKA T25 Digital Ultra-Turrax (IKA). This dispersant phase was then added dropwise to a continuous phase of 5% PVA and homogenized. The mixture was then added dropwise to 200 mL of 0.2% PVA and left stirring for 2 h to evaporate the solvent. NPs were collected by centrifugation at 12,000 rpm for 20 min at 4° C. and then washed 3 times with deionized water. The particles were lyophilized and stored at −20° C. The hydrodynamic diameter and surface charge of NPs were measured by a Malvern Zetasizer. A dispersion of NPs was filtrated through a 0.45 m Millipore filter into cuvettes prior to the measurements. Dynamic light scattering was measured by back-scattering at a detection angle of 1730 at the wavelength of 532 nm and the hydrodynamic radius was calculated using the Stokes-Einstein equation. The morphology of the NPs was observed by Hitachi S-4800 High Resolution scanning electron microscopy (SEM, Norcross). A dispersion of NPs in ethanol (2 L) was placed on the wafer substrate and dried at room temperature. The sample was mounted on the aluminum sample holder and then gold sputtered. The NPs were observed with an accelerating voltage of 15 kV at a working distance of 4 mm. Release of DIR and insulin was measured in the stomach-mimicking media. NPs were dispersed in the media (citrate buffer solution, pH 2.0) at 37° C. in the presence of pepsin (10 mg/mL). At each time point, NPs were centrifuged, and supernatant was collected to measure the amount of DIR released from the particles using a plate reader (λₑₓ 750 nm, λₑₘ 790 nm, SpectraMax M5, Molecular Devices). Insulin release was quantified by the BCA assay. Eudragit coated PLGA (PLGA®Eudragit) was prepared by dispersing PLGA in 5 wt % Eudragit solution and centrifugation.

Permeability of NPs though human intestinal epithelial cell layer. Caco-2 cells seeded at 7×10⁴ cells/cm² on 0.4 m pore transwell filters (Corning). Cells were grown to confluence and allowed to mature for approximately 30 days at 37° C. and 5% CO₂. Prior to performing permeability studies, the transepithelial electrical resistance (TEER) was measured using an epithelial voltohmmeter (EVOM™ Epithelial Volt/Ohm Meter, World Precision Instruments, Inc.). Confluent cell layers with TEER values greater than 300 Ω·cm² were used for permeability and cytotoxicity studies. A dispersion of 1 mg/mL DIR-loaded NPs or a solution containing an equivalent concentration of soluble DIR was prepared in phenol-free HBSS (Life Technologies) containing 25 mM glucose and added to the apical chamber of the transwell filter. HBSS in the basolateral chamber was sampled and replaced with fresh media at each time point. The rate of cumulative DIR transport to the basolateral chamber gave the flux, dQ/dt. The apparent permeability (Pₐₚₚ) was calculated by (1).

$${P}_{app} = \frac{dQ / dt}{{C}_{0} \times A} \qquad Eq , 1$$

where C₀ is the initial concentration of total DIR in the apical chamber and A is the area of the transwell filter.

TGR5 binding studies. The competitive binding of pUDCA, UDCA, and PLGA NPs to macrophages saturated with an Atto565-conjugated TGR5 antibody was performed. The cells (10¹ cells/well) in 96 well plates were incubated with an access amount (4 μg/mL) of the fluorescently labeled TGR5 antibody at 4° C. for 2 h and subsequently exposed to different concentration of NPs. At 2 h post incubation, the cells were washed three times with PBS and the number of Atto565-TGR5 antibodies bound on the cells were measured using a plate reader. The specific and non-specific k_{d} were calculated by non-linear fitting using a site saturation total binding equation, Y=Bₘₐₓ×X/(k_{d}+X)+NS×X, where Bₘₐₓ is the maximum specific binding, k_{d} is the equilibrium dissociation constant, and NS is the slope of nonspecific binding. To study valency dependent NP binding to TGR5 on pancreatic β cells (10⁶ cells/well), UDCA monomer was biotinylated and conjugated onto avidinated PLGA NP surface. PLGA (100 mg) in 2 mL chloroform was added dropwise to a mixture of avidin-palmitate in PBS (10 mg/2 mL) and 5% PVA 2 mL, and homogenized. UDCA was conjugated with biotin (1:1 molar ratio) using the EDC/NHS chemistry prior to immobilization of biotinylated UDCA (0, 50, 250, and 1000 ng/mL) to avidinated PLGA NPs (5 mg/mL). To prepare plated TGR5, recombinant TGR5 receptor (5 g/mL) was coated on the plate overnight and the non-specific binding sites were blocked using a protein blocking buffer. The TGR 5 receptors on the plates were incubated with an access amount (4 μg/mL) of the fluorescently labeled TGR5 antibody at 4° C. for 2 h and subsequently exposed to different concentration of NPs. At 2 h post incubation, the plates were washed three times with PBS and the number of Atto565-TGR5 antibodies bound on the receptors were measured using a plate reader.

Data was fit to the following competitive inhibition equation, (using Graphpad Prism), which gave an estimate of pUDCA at which 50% of the labeled antibody is competed off (EC₅₀) and its affinity constant (Kᵢ):

$$F = {F}_{Initial} + [ \frac{( {F}_{Final} - {F}_{Initial} )}{( 1 + {10}^{[ pUDCA ] - Log ( {EC}_{50} )}} ] \qquad Eq . 2$$

$$Log ( {EC}_{50} ) = Log [ {10}^{log {K}_{i} * ( 1 + \frac{{C}_{Anti}}{{K}_{D , Anti}} )} ] \qquad Eq . 3$$

where
F=Fluorescence change with competition against labeled TGR5 antibody;
   [pUDCA]=Concentration of pUDCA;
   Fᵢₙᵢₜᵢₐₗ=Upper plateau of fluorescence or initial fluorescence;
   F_{Final}=Lower plateau of fluorescence or final fluorescence;
   EC₅₀=Concentration of pUDCA that lowers the total fluorescence by 50%;
   Kᵢ=pUDCA affinity constant;
   C_{Anti}=Concentration of labeled anti-TGR5 antibody;
K_{D,Anti}=Affinity constant of the labeled anti-TGR5 antibody to TGR5 receptors estimated in the nanomolar range.

It was estimated that a shell of thickness equivalent to the molecular diameter of an UDCA can incorporate approximately 2000 UDCA monomers on a 350 nm diameter pUDCA particle.

Quantitation of cellular endocytosis and exocytosis rates of NPs. BMMs were seeded in a 96 well plate (10⁵ cells/well) and DIR-loaded pUDCA, PLGA, and PLGA/pUDCA blend NPs (100 μg/mL) were added to the media. Cells were incubated for 1, 3, and 6 h at 37° C., and endocytosis of NPs was measured using a plate reader. After washing the cells and replacing with new media, exocytosis of NPs was monitored at 37° C. or 4° C. by measuring released DIR-labeled NPs from BMMs to media over time. The method below is the first time such an analysis is applied to ascertain rates of endocytosis and exocytosis. A separate manuscript is in preparation discussing this methodology in greater detail, but briefly it is as follows:

The equilibrium endocytosis-exocytosis reaction can be simplified to:

$$[ P ] + [ C ] {⇌}_{{k}_{exo}}^{{k}_{endo}} [ PC ] \qquad Eq . 4$$

where=concentration of particles in the media (number of particles/mL)
   [C]=concentration of cells in the media (number of cells/mL)
   [PC]=concentration of particles associated with cells (number of particle-cell/mL)
   kₑₓₒ=rate of exocytosis (t⁻¹)
   k_{endo}=rate of endocytosis (([P]·t)⁻¹)
then,

$$\frac{d [ PC ]}{dt} = {k}_{endo} [ P ] [ C ] - {k}_{exo} [ PC ] \qquad Eq . 5$$

In terms of a signal reporting on the endocytosis and exocytosis process, which is a fluorescence signal associated with each process [S].

$$\frac{dS}{dt} = {k}_{endo} [ P ] S - ( {k}_{endo} [ P ] + {k}_{exo} ) S \qquad Eq . 6$$

This differential equation has a solution between the two extreme limits of no uptake to maximal uptake:

$$S = \frac{{k}_{endo} [ P ] {S}_{max} [ 1 - {e}^{- ( {k}_{endo} [ P ] + {k}_{exo} ) t} ]}{{k}_{endo} [ P ] + {k}_{e x o}} + {S}_{0} \qquad Eq . 7$$

S₀=signal at an arbitrary start time to

This analysis and fitting for k_{endo} and kₑₓₒ are done using two plots.

### Exocytosis Phase

$$\frac{dS}{dt} = - {k}_{e x o} S so Ln ( \frac{{S}_{0}}{{S}_{t}} ) = {k}_{exo} t \qquad Eq . 8$$

where St is the signal at any time (t)
S0 is the signal at an arbitrary time (t₀)

### Association Phase

The association phase is analyzed in terms of two plots:

$$dS / dt against S gives$$

$$Slope = - ( {k}_{endo} [ P ] + {k}_{e x o} ) , Intercept = {k}_{endo} [ P ] {S}_{max} at \frac{dS}{dt} = 0 \qquad Eq . 9$$

$$and Ln ( dS / dt ) against t gives$$

$$Slope = - ( {k}_{endo} [ P ] + {k}_{e x o} ) , \qquad Eq . 10$$

$$Intercept = Ln ( {k}_{endo} [ P ] {S}_{max )} ) at Ln ( \frac{dS}{dt} ) = 0.$$

Assumption: This analysis does not take account of particle re-uptake after exocytosis.

Flow cytometry and ELISA. CD44+CD8+ cells and CD4+CD25+Foxp3+ Tregs were acquired at 3- and 5-day post CY treatments for CY-induced mice. For spontaneous T1D animal model, T cells were collected at day 1 after the last NP dose. In both cases, pancreatic lymph nodes were harvested and processed using a 40 μm cell strainer. Cell surface markers were ascertained with fluorescent antibodies for CD8 (APC), CD44 (PE), CD4 (APC), and CD25 (Alexa Fluor-700) by incubating for 30 min at 4° C. Cells were then fixed, permeabilized, and stained for Foxp3 (PE) using the Foxp3 staining kit (eBiosciences). After the final wash, samples were immediately run on an LSR-II multicolor flow cytometer (BD Biosciences) and analyzed using FlowJo software (Tree Star). To study antigen-specific T cell responses, OVA-specific CD4+ cells were used in OTII co-culture assays. BMDCs (2.5×10⁴ cells/well, 96 well plate) were pretreated with pUDCA NPs for 24 h, washed, and then stimulated with LPS (10 ng/mL) and OVA (20 μg/mL) for 24 h, followed by co-culture with OTII CD4+ T cells (5×10⁴ cells/well, 96 well plate) for 3 days. Cell proliferation and cytokine production were then quantified.

BMMs (10⁵ cells/well, 96 well plate) were incubated with pUDCA NPs (50 μg/mL), UDCA monomer (50 μg/mL), PLGA (50 μg/mL), or PBS for 4 h and added with CpG (100 ng/mL). After 20 h, media supernatant was collected for IL-6, IL-10, and CCL1 readouts. Cells were stained for F4/80 (Alexa Fluor-647), CD86 (PE), and CD206 (FITC). After 3 washes with buffer (2% FBS in PBS), samples were fixed in 2% paraformaldehyde and run on an Attune NxT multicolor flow cytometer (Life Technologies). CD11c-F4/80+NP+ was used for NP tracking of macrophages with dye-loaded particles. The NP+ designation refers readout of the particle via fluorescence of loaded dye. Mice were fasted for 4 h and treated with labeled pUDCA NPs by oral gavage (500 mg/kg, 250 μL). After one day, pancreas, liver, lungs, and spleen were harvested and gently processed with a homogenizer and using a 40 μm cell strainer and plunger for separations of cells from debris. Cell surface markers were stained with fluorescent antibodies for F4/80 (Alexa Fluor-700) and CD11c (PE-Cy7) and measured by Attune NxT multicolor flow cytometer. All antibodies were diluted 1:500 for flow cytometry and cytokines (IL-6, IL-10, and IFNγ) were measured by ELISA (BD Biosciences).

### Biodistribution and Histology.

B6, NOD, or Nude mice were fasted for 4 h and treated with DIR- or coumarin 6-encapsulating NPs by oral gavage (50, 100, or 500 mg/kg, 250 μL). Free DIR or coumarin 6 (solubilized with 1% Tween 20) served as controls. Mice were sacrificed at time points of 4, 8, 12, or 24 h post gavage, and a Bruker molecular imaging instrument (Carestream Health, Inc.) was used to scan organs ex vivo to measure fluorescence intensity. Fluorescence data was fit to the one component exponential decay model: Y=Y_{f}+(Yₒ−e^{-kt}). DIR-loaded NPs formulated by pUDCA, PLGA or the mixture (50:50, w/w) were also intravenously (IV) administered (100 mg/kg, 50 μL) to mice via tail vein injection to compare their biodistribution with free dye. Macrophage Depletion Kit (Clodrosome®, Encapsula NanoSciences, 100 mg/kg, intraperitoneal (IP) injection) was used to deplete macrophages in B6 mice. For histology, pancreata from the mice orally received iron oxide-loaded pUDCA NPs were fixed in 10% neutral buffered formalin for histological analysis by hematoxylin and eosin (H&E) and Prussian blue stains. The stained sections were prepared by the Yale University Pathology Histology Service. Tissues were imaged on a Nikon TE-2000U microscope with a Nikon DS Fil color camera and NIS Elements AR software (version 2.30).

Experiments with diabetic animal models. NOD mice were intraperitoneally injected with CY (200 mg/kg) to induce acute type I diabetes (T1D) (FIG. 4A). After 24 h, the mice were orally gavaged with empty NPs, RAPA-loaded NPs (50, 100, and 500 mg NP/kg=40 mg RAPA/kg), soluble RAPA (40 mg/kg solubilized with 1% Tween 20), and saline. pUDCA_{RAPA} was orally administered at day 1 (Dose I) or twice on day 1 and 2 (Dose II). Blood glucose level was monitored using a blood glucose monitor (Trueresult® meter, Home Diagnostics, Inc.). Two readings (1 day apart) higher than 200 mg/dL were taken as an indication onset of T1D. pUDCA_{RAPA} were compared to “Gold Standard” insulin administration, insulin administered subcutaneously (SQ) or intraperitoneally (IP) using this model. After 7 consecutive injections of insulin or oral gavage of pUDCA_{INs} with an equivalent insulin dose, blood glucose was measured over the course of 25 days. Data was fitted using an operational receptor depletion model, Y=Basal+(Effectₘₐₓ Basal)/(1+operate), where operate=(((10^{log kA})+(10^{X}))/(10^{(log tau+X)}))ⁿ, Effectₘₐₓ is the maximum possible system response, Basal is the response in absence of agonist, kA is the agonist-receptor dissociation constant, and tau is the kinetics of lowering to half-maximal response.

For spontaneous T1D model, NOD mice were housed for approximately 2 months to allow them to naturally develop T1D. For spontaneous T1D model, NOD mice the same age (8 week old) were chosen for the study only when they became diabetic in the same week (16 week old, after 2 random tail vein blood glucose measurements of 200±15 mg/dL). The mice were orally treated with empty NPs, INS-loaded NPs (100 or 500 mg NP/kg and 285 mIU INS/kg) or free INS (285 mIU/kg) 7 times for 1 week to monitor glycosuria and body weight. Concentration of GLP-1 was measured using an ELISA kit (Invitrogen) from plasma. Swine were fasted for >12 hours with access to water and alloxan was administrated IV (250 mL, 150 mg/kg). At 10 day post alloxan treatment, the animals (n=3) were orally gavaged with pUDCA_{INS} (6.4 mg/kg equivalent to 100 mg/kg in mice) 7 times daily and monitored glucose readings using a continuous glucose monitoring system, DexCom 6 (Dexcom. Inc.). Calibration was done by ear stick with Relion Prime BG Meter (Relion) and further venous calibration was performed from whole blood samples by Antech Diagnostics.

Calculations for INS dose chosen in the spontaneous TID study. It is known that 1 international unit (IU) of INS is needed to drop blood glucose level of 50 mg/dL for human. To lower high blood glucose level (>400 mg/dL) below hyperglycemia threshold (200 mg/dL), at least 4 IUs of INS is required. That is 0.02 IUs of INS for mice according to the following equation (9).

$$HED = Animal NOAEL \times {( \frac{Weight of animal ( kg )}{Weight of human ( kg )} )}^{( 1 - 0. 67 )} \qquad Eq . 11$$

where HED=human equivalent dose (mg/kg), Animal NOAEL=No Observed Adverse Effect Levels for animal (mg/kg), 1 IU of INS=0.036 mg based on the World Health Organization conversion factor.

The loading of INS in pUDCA NPs was approximately 20 ng/mg of NP and it is 5.6×10⁴ IU/mg of NP. In order to feed 0.02 IUs of INS, 35 mg of pUDCA_{INS} NPs should be treated to single mouse. Therefore, diabetic mice were treated with 3 doses of 10 mg of NPs and continued to treat the mice with additional 4 doses to keep the glucose level low (total 0.04 IUs of INS and 70 mg of NPs).

Toxicology of pUDCA and UDCA. Acute toxicity studies were performed with 10-weekold, female B6 mice. Mice were orally dosed with pUDCA (100 and 500 mg/kg) and UDCA (100 mg/kg) at day 0. Kits from Teco diagnostic were used at day 3, 5, and 7 for analysis of serum concentrations of alkaline phosphatase, alanine transferase, total bilirubin, and blood urea nitrogen concentrations. EDTA anticoagulated blood was analyzed by a Hemavet blood counter (Drew Scientific).

Statistics. All statistical analyses were performed using GraphPad Prism software (version 7.01). Experimental comparisons with multiple groups used ANOVA analysis with Bonferroni's post test or Two-tailed Student's t tests were performed. Log-rank test and χ² statistical analysis was performed for survival data. A P value of 0.05 or less was considered statistically significant.

### Example 2. PUDCA is a Carrier and More than Additive Metabolic/Immunomodulatory Drug

### Materials and Methods

Materials and Methods are as described above.

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO).

The same experiment was performed in the presence of TGR5 antagonist, triamterene (50 μg/mL) as a control to differentiate inherent insulin production from the cells without TGR5 activation and used to normalize the results.

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO). The same experiment was performed in the presence of TGR5 antagonist, triamterene (50 μg/mL) as a control to differentiate inherent insulin production from the cells without TGR5 activation and used to normalize the results.

Anti CD28 5 μg/mL soluble and anti CD3, 10 ug/mL plated were used to stimulate OTII CD4+ T cells (5×10⁴ cells/well, 96 well plate) for 3 days. IFNγ were measured by ELISA (BD Bioscience).

### Results

pUDCA functions as a drug carrier but is also intrinsically therapeutic. With pUDCA_{INS}, blood glucose control is thus a convolution of direct insulin provision to the blood pool as well as intrinsic therapeutic benefit as a result of strong ligation of BA receptors. The extent to which insulin is made bioavailable from the material in both the pancreas and the blood pool is shown in FIGS. 6H and 61. First, consistent with earlier studies of biodistribution of orally ingested dye loaded pUDCA, insulin provisioned by pUDCA first appears higher in the pancreas, then naturally flows to the blood pool, highlighting the notion that pUDCA mediated delivery of insulin occurs in a physiologically relevant manner where it originates from the pancreas then made available in the blood. At 4, 8, 24 h post oral gavage of pUDCA_{INS}, increased pancreatic and blood insulin from pUDCA compared to delivery via PLGA or injection of soluble insulin was observed.

Second, pUDCA's inherent therapeutic character is underscored by the fact that, with no encapsulated insulin, it increased endogenous glucagon-like peptide (GLP-1) secretion and insulin production (FIGS. 6J and 6K). GLP-1, an incretin, has insulinotropic activity and can decrease blood sugar levels by promoting endogenous insulin secretion. The fact that GLP-1 secretion from intestinal enteroendocrine L cells is abrogated upon blockade of the TGR5 receptor in vivo after pUDCA ingestion emphasizes the role of pUDCA in binding to TGR5 and induction of therapeutic effects that go beyond insulin delivery alone. Indeed, beyond the metabolic control of insulin, binding to TGR5 resulted in T cell frequency immunomodulation with a reduction of CD44+CD8+ T cell frequency and an increase in Tregs in pancreatic lymph nodes of animals treated with pUDCA (FIGS. 6L and 6M), which is consistent with T1D prevention studies (FIGS. 4F-4I).

Both UDCA and pUDCA were able to induce more insulin secretion from pancreatic β cells compared to PLGA particles (FIG. 3M).

A reduction in interferon gamma (IFNγ) was also observed upon exposure of T cells to pUDCA followed by engagement of dendritic cells presenting antigens (FIG. 3D), or upon directly treating the CD4+ T cells with pUDCA and stimulated with anti-CD3 and anti-CD28 (FIG. 3N) and phenotypic skewing of macrophages from M1 to M2 (FIGS. 6N and 60, FIG. 3E). The anti-inflammatory function of pUDCA thus spans multiple immunological mechanisms ranging from innate to adaptive immunity and is consistent with prior reports exemplifying the various therapeutic and immunomodulatory facets of the monomer UDCA in different disease states. pUDCA is, however, a significantly potent format of UDCA and as such introduces mechanisms and modalities of function not possible with UDCA alone as demonstrated in FIGS. 6A-60.

### Example 3. Prevention of T1D: Validation of RAPA-Loaded pUDC

Given the biodistribution properties and the potential role of pUDCA in binding TGR5 with high avidity, leading to therapeutic agonistic effect in induction of an anti-inflammatory response, the role of pUDCA in the prevention of T1D was investigated. Two T1D animal models were utilized: the chemically inducible pancreatic inflammation using cyclophosphamide (CY) for the prevention study (FIGS. 4A-4I) and the spontaneous murine nonobese diabetic (NOD) mouse model for treatment of T1D (FIGS. 6A-60). The chemically inducible model was utilized to achieve initial control over disease pathophysiology and hence selection of optimal time for prophylactic intervention.

### Materials and Methods

Materials and methods are as described above.

pUDCA and its monomer UDCA, poly(lithocholic acid) (pLCA) and poly(deoxycholic acid) (pDCA) were all compared in this study. While LCA and DCA are known pro-inflammatory and carcinogenic agents, they are strong natural agonists of TGR5, and were used for comparison to pUDCA and its monomer UDCA in prevention of T1D (FIG. 4A). CY (200 mg/kg) was intraperitoneally (IP) injected to induce diabetic animals and the animals were administered two doses at 500 mg/kg on day 1 and 2. Blood glucose levels were then monitored for 30 days.

RAPA is a macrolide mTOR suppressor with immunosuppressive effects involving reduction of T and B cell sensitivity to interleukin-2 (IL-2)51. pUDCA encapsulating RAPA (pUDCA_{RAPA}, 0.08 mg per mg of NP) was administered once at one day post induction (Dose I) or twice on day 1 and 2 (Dose II) (FIG. 4A).

### Results

Strikingly, in comparison with pLCA, pDCA and even UDCA, pUDCA oral ingestion resulted in the lowest blood glucose levels and least frequent conversion to a diabetic state without any loaded drugs (FIGS. 4B and 4C). pUDCA's benefit was greater than pLCA and pDCA despite LCA and DCA being two of the strongest TGR5 agonists. This could be mediated by differences in their hydrophobicity and lower bioavailability compared to pUDCA, or because LCA and DCA are pro-inflammatory. It is also possible that the high original affinity of LCA and DCA reduced avidities as a result of multivalency; a ubiquitous biophysical phenomenon that can decrease intrinsic affinities with higher valencies due to competition for limited BA receptor sites. In general, pUDCA amplified the therapeutic effects of UDCA, and the mechanisms for this amplified response and potential for synergistic effects with encapsulated drug were examined.

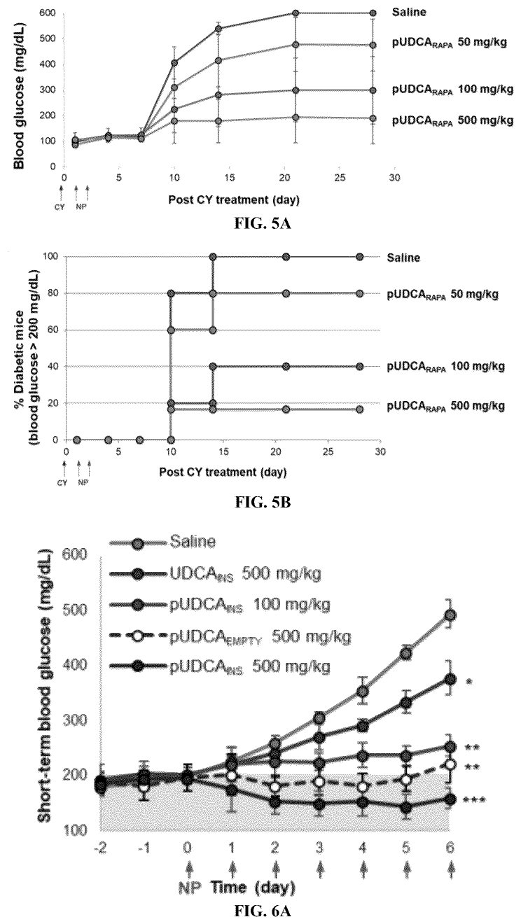

While 60% of drug-induced mice showed high blood glucose levels (>200 mg/dL) with PLGA treatment at 10 days post-induction, glucose levels were attenuated for 30 days by RAPA loaded pUDCA NPs in a dose-dependent manner (FIGS. 4D and 4E, 5A and 5B). PLGA_{RAPA} and RAPA alone did not attenuate blood glucose to the same level and 60% animals developed T1D (FIGS. 4D and 4E). Analysis of the CD4+ and CD8+ T cell response in the pancreatic draining lymph nodes of surviving animals (FIGS. 4F-4I) showed that the frequency of activated CD8+ T cells (CD44+) in treated mice was decreased with pUDCA_{RAPA} compared to saline (63% and 88% decrease for Dose I and II, respectively at day 5). Furthermore, there was a simultaneous increase in the frequency of regulatory (CD4+CD25+Foxp3+) T cells (Tregs) with pUDCA_{RAPA} compared to saline (2.4 and 9.7 fold increase for Dose I and II, respectively).

### Example 4. Multimodal Treatment of T1D: Preclinical Validation of Orally Administered Insulin pUDCA

Treatment of established disease is a more challenging proposition because of the spontaneous nature of the pathology and the heterogeneity of disease manifestation over time. Since insulin is a “gold-standard” systemic therapeutic, the ability of insulin loaded pUDCA (pUDCA_{INS}) in abrogating established disease was tested in NOD T1D animal models. This is a model with a long established history as a human equivalent diabetes model in mice with well-defined expectations in terms of effects of soluble insulin in treatment.

### Materials and Methods

NOD mice, with blood glucose levels about 200 mg/dL, were orally gavaged with two different doses of pUDCA_{INS} (100 or 500 mg/kg, corresponding to 2 and 10 mg of NP per animal, respectively) (FIG. 6A). Because of the weak agonist nature of the monomer UDCA, comparison with pUDCA was conducted at the higher dose (500 mg/kg). Dosing was ascertained based on a rough estimation of the predicted amount of insulin needed to restore blood glucose levels to normal (below 200 mg/dL); a cumulative dose of 40 mIU per mouse per group corresponding to 10 mg of particles per mouse per day for seven consecutive days) (FIGS. 6A-6E).

### Results

Glucose levels decreased after 2 doses of pUDCA encapsulating a total of 0.011 IUs of insulin; reducing plasma glucose levels to or below the 200 mg/dL threshold for diabetes (FIG. 6A). pUDCA was significantly more effective in reducing blood glucose levels over the seven day period compared to its monomer counterpart. With humans, 1 IU of insulin generally equates to a blood glucose lowering of 50 mg/dL. This result demonstrates that with a dose that is nearly 100 fold lower, renormalization of blood glucose level can be achieved in the short-term with pUDCA in a more potent manner than UDCA.

TABLE 3
Time needed for 50% glucose lowering.

| | Route | T |
|---|---|---|
| Saline | Oral | 9474.0 |
| | Oral | 4318.0 |
| Soluble INS | SC | 7.4 |
| | IP | 0.3 |
| pUDCA_{INS} | Oral | approximately 0 |

### Example 5. PUDCA and Antigen-Specific Tolerance with Model Antigen Ovalbumin

### Materials and Methods

Materials and methods are as described above.

The pUDCA NPs tested were either empty, contained only the antigen OVA (ovalbumin), or a combination of OVA and RAPA.

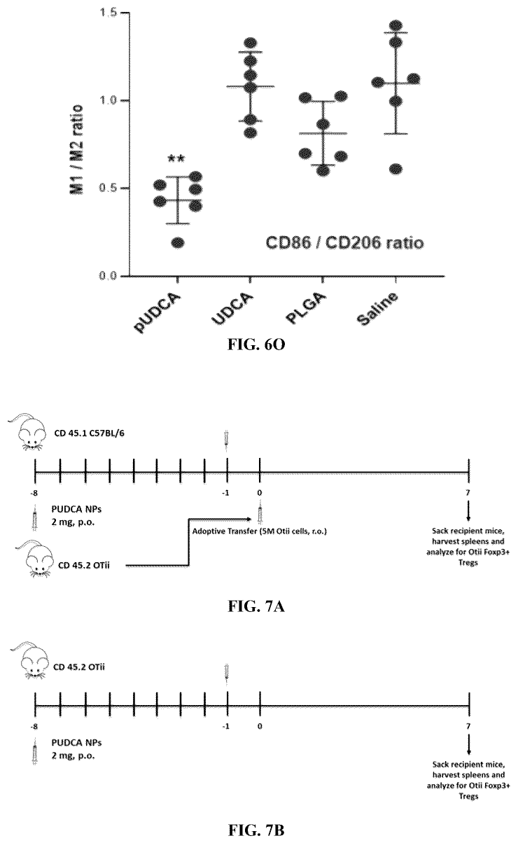

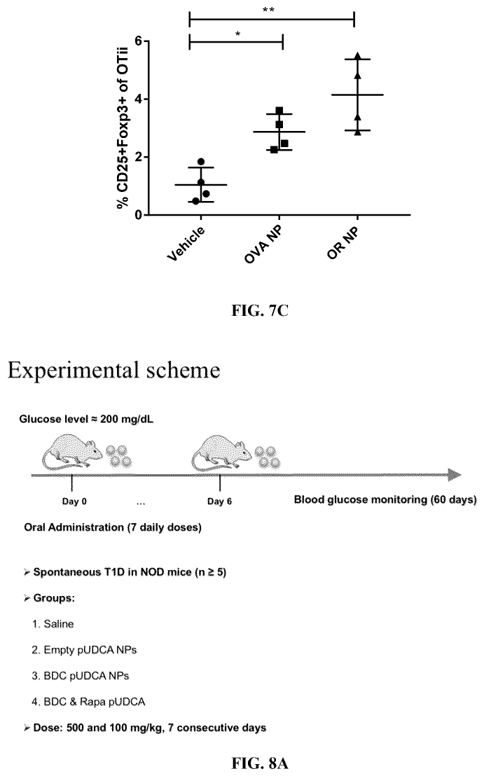

This study used two groups of mice: Group A for detecting the efficacy with OTii adoptive transfer, and Group B for evaluating efficacy in OTii Mice (no cell transfer). The experimental set up is shown in FIGS. 7A and 7B.

### Results

The results are shown in FIG. 7C.

The results are also summarized in Table 4 below.

TABLE 4
Change in percent CD25+FoxP3− and CD25+FoxP3+
cells from Group A and Group B mice (n = 2).

| | | | | | |
|---|---|---|---|---|---|
| PUDCA | PUDCA | OVA | OVA | O-Rapa | O-Rapa |
| NP | NP | NP | NP | NP | NP |
| GRP A | GRP B | GRP A | GRP B | GRP A | GRP B |

CD25+FoxP3−

| | | | | | | |
|---|---|---|---|---|---|---|
| (%) | 2.12 | 1.38 | 22.73 | 25.89 | 2.34 | 3.88 |

CD25+FoxP3+

| | | | | | | |
|---|---|---|---|---|---|---|
| (%) | 11.65 | 9.45 | 11.6 | 10.72 | 15.51 | 13.45 |

### Example 6. Prevention of T1D: Validation of RAPA-Loaded pUDCA

Given the biodistribution properties and the potential role of pUDCA in binding TGR5 with high avidity, leading to therapeutic agonistic effect in induction of an anti-inflammatory response, the role of pUDCA in the prevention of T1D was investigated.

Treatment of established disease is a more challenging proposition because of the spontaneous nature of the pathology and the heterogeneity of disease manifestation over time. Since insulin is a “gold-standard” systemic therapeutic, the ability of insulin loaded pUDCA (pUDCA_{INS}) in abrogating established disease was tested in NOD T1D animal models. This is a model with a long established history as a human equivalent diabetes model in mice with well-defined expectations in terms of effects of soluble insulin in treatment.

### Materials and Methods

Materials and methods were as described above.

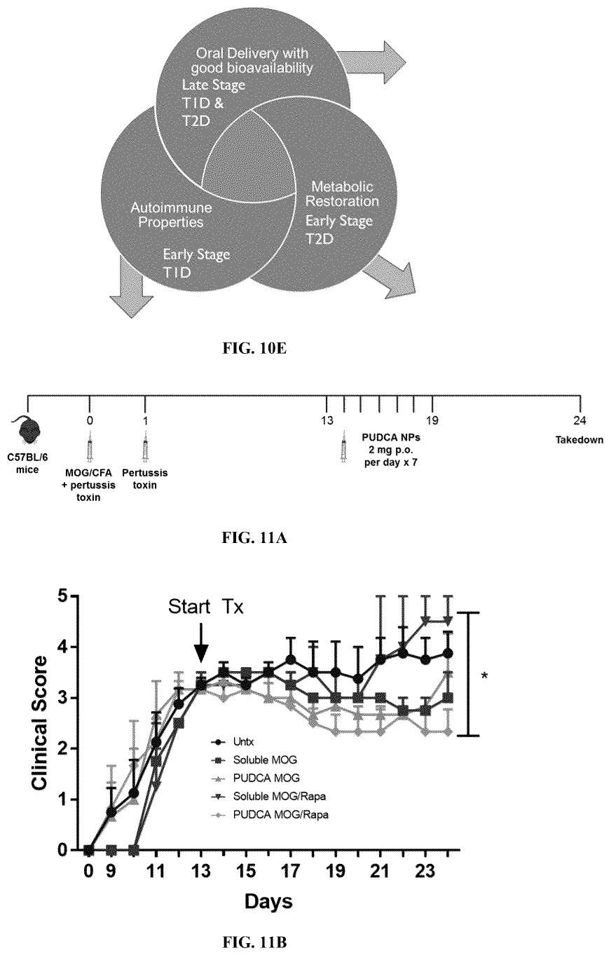

Two T1D animal models were utilized: the chemically inducible pancreatic inflammation using cyclophosphamide (CY) for the prevention study (FIGS. 9A-9I) and the spontaneous murine nonobese diabetic (NOD) mouse model for treatment of T1D (FIGS. 11A-110 and Table 6). The chemically inducible model was utilized to achieve initial control over disease pathophysiology and hence selection of optimal time for prophylactic intervention.

pUDCA and its monomer UDCA, poly(lithocholic acid) (pLCA) and poly(deoxycholic acid) (pDCA) were all compared in this study. While LCA and DCA are known pro-inflammatory and carcinogenic agents, they are strong natural agonists of TGR5, and were used for comparison to pUDCA and its monomer UDCA in prevention of T1D (FIG. 9A).

CY (200 mg/kg) was intraperitoneally (IP) injected to induce diabetic animals and the animals were administered two doses at 500 mg/kg on day 1 and 2. Blood glucose levels were then monitored for 30 days.

NOD mice, with blood glucose levels about 200 mg/dL, were orally gavaged with two different doses of pUDCA_{INS} (100 or 500 mg/kg, corresponding to 2 and 10 mg of NP per animal, respectively) (FIG. 11A). Because of the weak agonist nature of the monomer UDCA, comparison with pUDCA was conducted at the higher dose (500 mg/kg). Dosing was ascertained based on a rough estimation of the predicted amount of insulin needed to restore blood glucose levels to normal (below 200 mg/dL, see METHODS); a cumulative dose of 40 mIU per mouse per group corresponding to 10 mg of particles per mouse per day for seven consecutive days) (FIGS. 11A-11E).

RAPA is a macrolide mTOR suppressor with immunosuppressive effects involving reduction of T and B cell sensitivity to interleukin-2 (IL-2)51. pUDCA encapsulating RAPA (pUDCA_{RAPA}, 0.08 mg per mg of NP) was administered once at one day post induction (Dose I) or twice on day 1 and 2 (Dose II) (FIG. 9A).

### Results

Strikingly, in comparison with pLCA, pDCA and even UDCA, pUDCA oral ingestion resulted in the lowest blood glucose levels and least frequent conversion to a diabetic state without any loaded drugs (FIGS. 9B and 9C).

TABLE 5
Time needed for 50% glucose lowering.

| | Route | T |
|---|---|---|
| Saline | Oral | 9474.0 |
| Soluble INS | Oral | 4318.0 |
| | SC | 7.4 |
| | IP | 0.3 |
| pUDCAᵢₙₛ | Oral | approximately 0 |

pUDCA's benefit was greater than pLCA and pDCA despite LCA and DCA being two of the strongest TGR5 agonists. This could simply be mediated by differences in their hydrophobicity and lower bioavailability compared to pUDCA, as suggested in FIG. 3A, or because LCA and DCA are pro-inflammatory. It is also possible that the high original affinity of LCA and DCA reduced avidities as a result of multivalency; a ubiquitous biophysical phenomenon that can decrease intrinsic affinities with higher valencies due to competition for limited BA receptor sites. In general, pUDCA amplified the therapeutic effects of UDCA, and the mechanisms for this amplified response and potential for more than additive effects with encapsulated drug were examined.

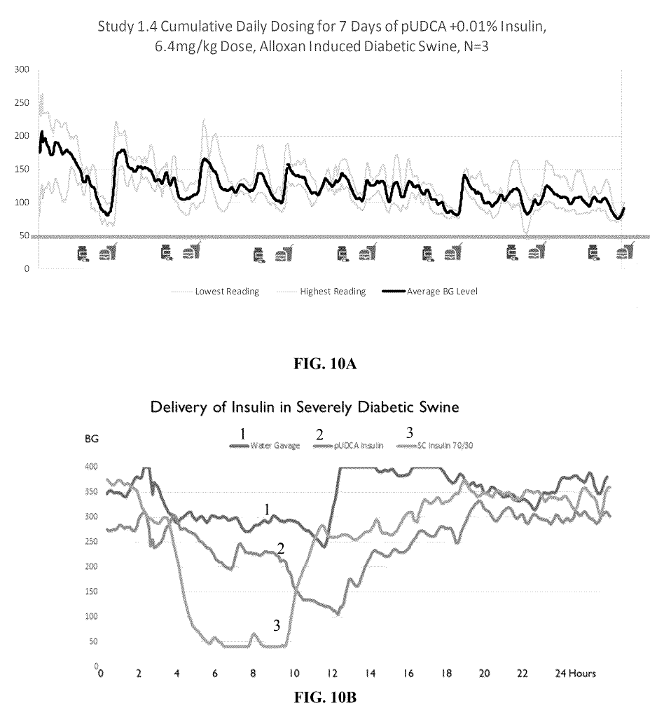

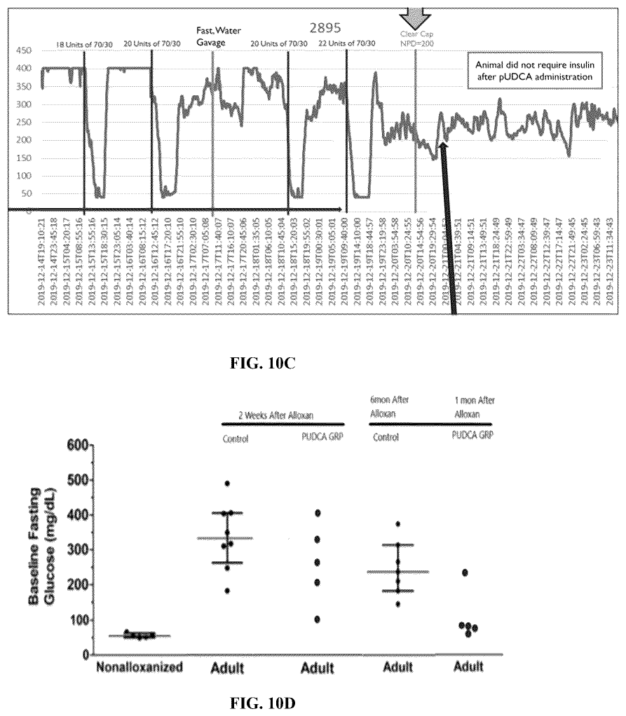

While 60% of drug-induced mice showed high blood glucose levels (>200 mg/dL) with PLGA treatment at 10 days post-induction, glucose levels were attenuated for 30 days by RAPA loaded pUDCA NPs in a dose-dependent manner (FIGS. 10A and 10B). PLGA_{RAPA}, or RAPA alone did not attenuate blood glucose to the same level and 60% animals developed T1D. Analysis of the CD4+ and CD8+ T cell response in the pancreatic draining lymph nodes of surviving animals showed that the frequency of activated CD8+ T cells (CD44+) in treated mice was decreased with pUDCA_{RAPA} compared to saline (63% and 88% decrease for Dose I and II, respectively at day 5).

Furthermore, there was a simultaneous increase in the frequency of regulatory (CD4+CD25+Foxp3+) T cells (Tregs) with pUDCA_{RAPA} compared to saline (2.4 and 9.7 fold increase for Dose I and II, respectively).

Oral administration of pUDCA/insulin regresses hyperglycemia in the short-term and induces long lasting therapeutic immunomodulation in spontaneous T1D in NOD mice (FIG. 8A), when administered BDC and RAPA-pUDCA (FIGS. 8B-8E).

Glucose levels decreased after 2 doses of pUDCA encapsulating a total of 0.011 IUs of insulin, reducing plasma glucose levels to or below the 200 mg/dL threshold for diabetes. pUDCA was significantly more effective in reducing blood glucose levels over the seven day period compared to its monomer counterpart. With humans, 1 IU of insulin generally equates to a blood glucose lowering of 50 mg/dL. This result demonstrates that with a dose that is nearly 100 fold lower, renormalization of blood glucose level can be achieved in the short-term with pUDCA in a more potent manner than UDCA.

### Example 7. Insulin-Loaded pUDCA NPs Rapidly Reversed Alloxan-Induced Diabetes in Adult Ossabaw Swine

### Materials and Methods

Materials and methods are as described above.

In Alloxan-induced diabetes, glucokinase is inhibited (short to medium term), and selective necrosis of beta cells due to reactive oxygen species ROS formation (long term) is observed. Animals are “stable” and diabetic after 10 days (FIGS. 9A and 9B).

The goal of this study was to test the therapeutic effect beyond possible Alloxan auto-recovery fluctuations (about 10% in a 10-day period). Three pigs were used for this study, pigs #2869, #2847, and #2832, and had alloxan-induced diabetes. The pigs received cumulative daily dose for 7 days of pUDCA with 0.01% insulin, 6.4 mg/kg dose.

### Results

Results in FIG. 10A show that a seven-day treatment of Alloxan-induced diabetic swine with insulin-containing pUDCA produced significant change in the average blood glucose levels over (FIGS. 10A and Table 6). There was a substantial change in blood glucose levels with oral pUDCA-insulin treatment when compared to treatment with subcutaneous insulin (FIGS. 10B and 10C). FIG. 10C shows that a single dose of pUDCA-insulin (NDP=200) was sufficient to eliminate the need for repeat SC insulin injections. FIG. 10E shows that pUDCA NPs offer diabetic care and treatment from three points: oral delivery with good bioavailability for treating late stage T1D and T2D, metabolic restoration for treating early stage T1D, and reduction in autoimmune reactivity for early stage T1D.

TABLE 6
Effect of pUDCA-insulin treatment on blood glucose levels
(BG, in mg/dL) in Alloxan-induced diabetic swine.

| | Day −1 | Day 7 | % Change |
|---|---|---|---|
| Average BG | 199 | 102 | −49% |
| Fasting BG | 144 | 81 | −44% |
| Post Prandial BG _{(0.5-4 hrs)} | 233 | 109 | −53% |

### Example 8. Antigen Specific Tolerance in Animal Models of Multiple Sclerosis

### Materials and Methods

The materials and methods are as described above.

The experimental scheme is shown in FIG. 11A. The immunizing and tolerizing antigen was myelin oligodendrocyte glycoprotein 35-55 (MOG). Therapeutic dosing, i.e. histological and physical evidence of disease. Traditional scoring, 0 to 5 reflecting limp tail to hind/front leg paralysis.

### Results

Oral administration of PUDCA NPs resulted in significantly reduced disease scores. PUDCA-MOG was effective but the addition of Rapa was most effective. Soluble Rapa with MOG was ineffective, demonstrating impact of the PUDCA platform (delivery, cytoprotective and anti-inflammatory effects) (FIG. 11B).

### Example 9. Antigen Specific Tolerance in Animal Models of Collagen-Induced Arthritis

### Materials and Methods

The materials and methods are as described above.

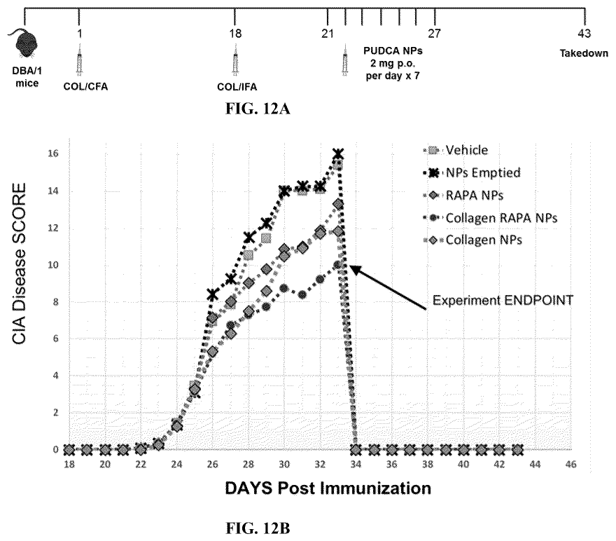

The experimental scheme is shown in FIG. 12A. The immunizing and tolerizing antigen was collagen (COL) for establishing animal models of rheumatoid arthritis. Semi-therapeutic dosing was used, i.e. histological evidence of disease. Traditional scoring, 0 to 4 per paw, max score 16 per mouse was used for collagen-induced arthritis (CIA) disease score.

Treatment started on day 21, after two doses of antigen challenge.

### Results

Oral administration of PUDCA NPs resulted in reduced disease scores. PUDCA-COL was effective but the addition of Rapa was most effective. PUDCA-Rapa showed some efficacy, likely reflecting synergistic anti-inflammatory activities. Model ended prematurely due to severe disease. Results are shown in FIG. 12B.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A formulation for inducing antigen-specific tolerance or non-specific decreased inflammation in a subject comprising
an effective amount of nanoparticles comprising bile acid esterified polymers having a molecular weight between about 800 and 240,000 Dalton (Da),
an immunomodulatory agent that decreases an immune response to an antigen, decreases inflammation or increases regulatory T cells, and, optionally, an antigen associated with an undesirable immune response.

2. The formulation of claim 1, wherein the bile acid esterified polymers:
(i) have a molecular weight between about 8000 and 20,000 Da, corresponding to a polymer of at least two bile acid monomers, or
(ii) are selected from the group consisting of polymeric ursodeoxycholic acid (pUDCA); polymeric lithocholic acid (pLCA); polymeric deoxycholic acid (pDCA); polymeric chenodeoxycholic acid (pCDCA); and polymeric cholic acid (pCA).

3. (canceled)

4. The formulation of claim 1, wherein the bile acid esterified polymers are pUDCA having a molecular weight between about 800 and 5 000 Da.

5. The formulation of claim 4, wherein the bile acid esterified polymers are pUDCA having as shown in Formula VII: 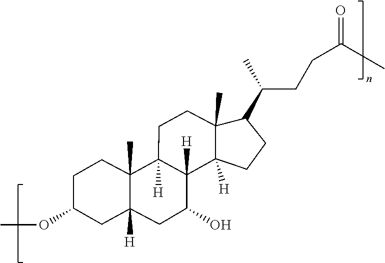
wherein n is a number between 2 and 20.

6. The formulation of claim 1, wherein the bile acid esterified polymers;
(i) form a surface on the nanoparticles comprising between 100 and 5000 bile acid monomers and have at least 1.5 fold greater affinity to bile acid receptors than respective monomers forming the bile acid esterified polymers; or
(ii) are linear and/or branched polymers.

7. (canceled)

8. The formulation of claim 1, wherein the immunomodulatory agent is selected from the group consisting of rapamycin (sirolimus) and analogs of rapamycin.

9. The formulation of claim 1 wherein the immunomodulatory agent:
(i) is an immunosuppressant; or
(ii) increases the number of regulatory T cells.

10. (canceled)

11. The formulation of claim 1, wherein the formulation comprises, or is in a kit comprising, a self-antigen, a disease-specific antigen, a species specific antigen, or an expression vector specific antigen.

12. The formulation of claim 1 comprising a diagnostic agent.

13. A method of inducing tolerance or decreasing an immune response in a subject comprising orally administering to the subject an effective amount of the formulation of claim 1, optionally wherein the effective amount of the formulation comprises between about 0.1 mg nanoparticles/kg and 1000 mg nanoparticles/kg body weight.

14. The method of claim 13, wherein the nanoparticles preferentially distribute to internal organs selected from the group consisting of heart, kidneys, spleen, lungs, liver, and pancreas in the absence of targeting molecules specific for of heart, kidneys, spleen, lungs, liver, or pancreas.

15. The method of claim 13, wherein the subject has an autoimmune or allergic disease selected from the group consisting of type 1 diabetes, systemic lupus erythematous, rheumatoid arthritis, multiple sclerosis, food allergies, environmental allergies, and diseases with anti-drug or nucleic acid antibodies (ADA).

16. (canceled)

17. The method of claim 13 wherein the subject has type 1 diabetes, and the method comprises orally administering to a subject in need thereof an effective amount of the formulation comprising an immunosuppressant or tolerance inducing agent to decrease blood glucose.

18. The method of claim 17, wherein the nanoparticles comprise rapamycin and insulin.

19. The method of claim 17, wherein the formulation is administered for a period of at least one week, at least two weeks, or at least three weeks.

20. The method of claim 17, wherein the formulation is administered once a day.

21. The method of claim 17, wherein the subject maintains healthy blood glucose for at least about three days, about five days, about one week, about two weeks, about one month, or more, following cessation of administering the formulation of claim 1.

22. The method of claim 17, wherein the method:
(i) increases the number of regulatory T cells in the subject; or
(ii) induces a tolerogenic phenotype in the subject.

23. (canceled)

24. The method of claim 13 wherein the subject;
(i) has systemic lupus erythematosus, and the method comprises orally administering to a subject in need thereof an effective amount of the formulation of claim 1 to decrease one or more symptoms of the disease;
(ii) has rheumatoid arthritis, and the method comprises orally administering to a subject in need thereof an effective amount of the formulation of claim 1 to decrease pain;
(iii) has multiple sclerosis, and the method comprises orally administering to a subject in need thereof an effective amount of the formulation of claim 1 to decrease one or more symptoms of the disease; or
(iv) has an allergy, and the method comprises orally administering to a subject in need thereof an effective amount of the formulation of claim 1 to decrease the allergic response.

25. (canceled)

26. (canceled)

27. The method of claim 13 wherein the drug has or is at risk of developing anti-drug antibodies comprising orally administering to a subject in need thereof an effective amount of the formulation of claim 1 to induce tolerance to the drug.

28. (canceled)
